(19) United States
(12) Patent Application Publication
(10) Pub. No.: US 2014/0105891 A1
(43) Pub. Date: Apr. 17, 2014

(54) TREATMENT OF CANCER

(71) Applicant: CERULEAN PHARMA INC., Cambridge, MA (US)

(72) Inventors: Edward G. Garmey, Boston, MA (US); Scott Eliasof, Lexington, MA (US)

(73) Assignee: CERULEAN PHARMA INC., Cambridge, MA (US)

(21) Appl. No.: 14/046,513

(22) Filed: Oct. 4, 2013

Related U.S. Application Data
(60) Provisional application No. 61/710,272, filed on Oct. 5, 2012.
(60) Provisional application No. 61/721,860, filed on Nov. 2, 2012.

(51) Int. Cl. A61K 47/48; A61K 31/44; A61K 39/395

(52) U.S. Cl. CPC A61K 47/4823; A61K 39/3955; A61K 31/44
USPC 424/133.1; 514/58

(57) ABSTRACT

Provided are methods relating to compositions that include a CDP-topoisomerase inhibitor, e.g., a CDP-camptothecin or camptothecin derivative conjugate, e.g., CRLX101.

This application claims priority to U.S. Ser. No. 61/710,272 filed Oct. 5, 2012 and U.S. Ser. No. 61/721,860 filed Nov. 2, 2012, the entire contents of each of these applications is incorporated herein by reference.

### BACKGROUND OF THE INVENTION

Drug delivery and dosing of small molecule therapeutic agents, such as camptothecin, can be problematic due to a number issues including half-life, toxicity, distribution etc.

### SUMMARY OF THE INVENTION

In one aspect, the disclosure features, a method of treating a proliferative disorder, e.g., a cancer, in a subject. The method comprises:

administering a CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, in combination with an angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, and the angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib. sunitinib, pazopanib or brivanib, or an antibody against VEGF receptor, are administered on the same dosing schedule, e.g., the topoisomerase inhibitor conjugate, particle or composition is administered on the same day, e.g., within 1 hour, 2 hours, 3 hours, 5 hours, 10 hours, 12 hours, 15 hours, 18 hours, 21 hours, 24 hours, as the angiogenesis inhibitor.

In one embodiment, the method further comprises administering one or more additional doses of the topoisomerase inhibitor conjugate, particle or composition and one or more additional doses of the angiogenesis inhibitor, wherein the additional dose or additional doses of the topoisomerase inhibitor conjugate, particle or composition, e.g., CRLX101, is administered on the same dosing schedule as the one or more additional dose or doses of the angiogenesis inhibitor e.g., where each subsequent administration of the topoisomerase inhibitor, conjugate or particle, e.g., CRLX101, is provided, independently, between 9, 10, 11, 12, 13, 14, 15, 16 or 17 days, e.g., 14 days, after the previous, e.g., the initial, administration, and where each subsequent administration of the angiogenesis inhibitor, e.g., a VEGF pathway inhibitor, e.g., a VEGF pathway inhibitor described herein, a VEGF pathway inhibitor, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor, is provided, independently, between 9, 10, 11, 12, 13, 14, 15, 16, or 17 days, e.g., 14 days, after the previous, e.g., the initial, administration.

In one embodiment, each subsequent administration of the topoisomerase inhibitor, conjugate or particle, e.g., CRLX101, is provided, independently, between 17, 18, 19, 20, 21, 22, 23, 24, 25, 26, 27, 28, 29, 30, or 31 days, e.g., 21 days, after the previous, e.g., the initial, administration, and each subsequent administration of the angiogenesis inhibitor, e.g., a VEGF pathway inhibitor, e.g., a VEGF pathway inhibitor described herein, a VEGF pathway inhibitor, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor, is provided, independently, between 17, 18, 19, 20, 21, 22, 23, 24, 25, 26, 27, 28, 29, 30, or 31 days, e.g., 21 days, after the previous, e.g., the initial, administration.

In one embodiment, the CDP-topoisomerase inhibitor conjugate, particle or composition, e.g., CRLX101, is administered at a dose of 3 mg/m², 4 mg/m², 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m² or 30 mg/m², (wherein the dosage is expressed in mg of drug, as opposed to mg of conjugate) (e.g., 9 mg/m², 10 mg/m², 11 mg/m², 12 mg/m², 13 mg/m², 14 mg/m² or 15 mg/m²), and each subsequent administration is independently administered at a dose of 3 mg/m², 4 mg/m², 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m² or 30 mg/m² (e.g., 9 mg/m², 10 mg/m², 11 mg/m², 12 mg/m², 13 mg/m², 14 mg/m² or 15 mg/m²), e.g., on a dosing schedule described herein, e.g., each subsequent administration of the topoisomerase inhibitor, conjugate or particle, e.g., CRLX101, is provided, independently, between 9, 10, 11, 12, 13, 14, 15, 16 or 17 days, e.g., 14 days. In an embodiment, CRLX101 is provided at 12-17 mg/m²/administration, e.g., 12-15 mg/m²/administration, e.g., 12 mg/m² or 15 mg/m².

In one embodiment, the angiogenesis inhibitor, e.g., bevacizumab, is administered at a dose of 15 mg/kg or less, e.g., 10 mg/kg or less, e.g., less than 10 mg/kg, e.g., 8 mg/kg, 7 mg/kg, 6 mg/kg, 5 mg/kg, 4 mg/kg, 3 mg/kg, or 2 mg/kg, and each subsequent administration is independently administered at a dose of 15 mg/kg or less, e.g., 10 mg/kg or less, e.g., less than 10 mg/kg, e.g., 8 mg/kg, 7 mg/kg, 6 mg/kg, 5 mg/kg, 4 mg/kg, 3 mg/kg, or 2 mg/kg, e.g., on a dosing schedule described herein, e.g., each subsequent administration of the angiogenesis inhibitor, e.g., bevacizumab, is provided, independently, between 9, 10, 11, 12, 13, 14, 15, 16 or 17 days, e.g., 14 days, after the previous dose.

In one embodiment, the angiogenesis inhibitor, e.g., aflibercept, is administered at a dose of 8 mg/kg or less, e.g., 6 mg/kg or less, e.g., 4 mg/kg, 3 mg/kg, 2 mg/kg, and each subsequent administration is independently administered at a dose of 8 mg/kg or less, e.g., 6 mg/kg or less, e.g., 4 mg/kg, 3 mg/kg, 2 mg/kg, e.g., on a dosing schedule described herein, e.g., each subsequent administration of the angiogenesis inhibitor, e.g., aflibercept, is provided, independently, between 9, 10, 11, 12, 13, 14, 15, 16 or 17 days, e.g., 14 days, after the previous dose.

In another embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, is administered prior to the administration of the angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor. For example, in one embodiment, CRLX101 is administered first, followed by subsequent administration of the angiogenesis inhibitor, e.g., a VEGF pathway inhibitor, e.g., a VEGF pathway inhibitor described herein, e.g., a VEGF inhibitor (e.g., aflibercept) or an antibody against VEGF, e.g., bevacizumab; or a VEGF receptor inhibitor (e.g., sorafenib. sunitinib, pazopanib or brivanib, or an antibody against VEGF receptor).

In one embodiment, the CRLX101 is administered at a dose described herein. In another embodiment, the method further comprises administering one or more additional dosages of CRLX101, e.g., 1, 2, 3, 4, 5, 6, 7, 8, 9, 10, 11, 12, 13, 14, 15 additional dosages of CRLX101. In some embodiments, the CRLX101 can be administered, independently, between 9, 10, 11, 12, 13, 14, 15, 16, or 17 days, e.g., 14 days, after the previous, e.g., the initial, administration of CRLX101.

In some embodiments, the CRLX101 can be administered, independently, between 17, 18, 19, 20, 21, 22, 23, 24, 25, 26, 27, 28, 29, 30, or 31 days, e.g., 21 days, after the previous, e.g., the initial, administration of CRLX101. After administration of CRLX101, the angiogenesis inhibitor, e.g., a VEGF pathway inhibitor, e.g., a VEGF pathway inhibitor described herein, a VEGF pathway inhibitor, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor, can be administered. In one embodiment, the angiogenesis inhibitor, e.g., a VEGF pathway inhibitor, e.g., a VEGF pathway inhibitor described herein, e.g., a VEGF inhibitor (e.g., aflibercept) or an antibody against VEGF, e.g., bevacizumab at a dose described herein. In one embodiment, one or more subsequent administrations of the angiogenesis inhibitor, e.g., a VEGF pathway inhibitor, e.g., a VEGF pathway inhibitor described herein, can be administered, independently, between 9, 10, 11, 12, 13, 14, 15, 16, or 17 days, e.g., 14 days, after the previous, e.g., the initial, administration of the angiogenesis inhibitor, e.g., a VEGF pathway inhibitor, e.g., a VEGF pathway inhibitor described herein.

In one embodiment, one or more subsequent administrations of the angiogenesis inhibitor, e.g., a VEGF pathway inhibitor, e.g., a VEGF pathway inhibitor described herein, can be administered, independently, between 17, 18, 19, 20, 21, 22, 23, 24, 25, 26, 27, 28, 29, 30, or 31 days, e.g., 21 days, after the previous, e.g., the initial, administration of the angiogenesis inhibitor, e.g., a VEGF pathway inhibitor, e.g., a VEGF pathway inhibitor described herein.

In one embodiment, the angiogenesis inhibitor, e.g., the VEGF pathway inhibitor, e.g., a VEGF pathway inhibitor described herein, is administered 1 hour, 2 hours, 3 hours, 5 hours, 10 hours, 15 hours, 18 hours, 21 hours, or 1, 2, 3, 4, 5, 6, 7, 8, 9 or 10 days after the administration of the CDP-topoisomerase inhibitor conjugate, particle or composition, e.g., CRLX101. In one embodiment, the initial administration of the angiogenesis inhibitor, e.g., the VEGF pathway inhibitor, e.g., a VEGF pathway inhibitor described herein, is administered on the same day, or within 1, 2, 3, 4, 5 days from the one of more subsequent doses of the topoisomerase inhibitor conjugate, particle or composition, e.g., CRLX101.

In another embodiment, the angiogenesis inhibitor, e.g., a VEGF pathway inhibitor, e.g., a VEGF pathway inhibitor described herein, a VEGF pathway inhibitor, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor, can be administered prior to the administration of the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101. For example, in one embodiment, the angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor, can be administered first, followed by subsequent administration of the CDP-topoisomerase inhibitor conjugate, particle or composition, e.g., CRLX101.

In one embodiment, the angiogenesis inhibitor, e.g., a VEGF pathway inhibitor, e.g., a VEGF pathway inhibitor described herein, e.g., a VEGF inhibitor, e.g., e.g., aflibercept or bevacizumab, can be administered at a dose described herein. In one embodiment, the method further comprises administering one or more additional dose of the angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor, e.g., 1, 2, 3, 4, 5, 6, 7, 8, 9, 10, 11, 12, 13, 14, 15 additional dosages.

In one embodiment, the angiogenesis inhibitor, e.g., a VEGF pathway inhibitor, e.g., a VEGF pathway inhibitor described herein, can be administered, independently, between 9, 10, 11, 12, 13, 14, 15, 16, or 17 days, e.g., 14 days, after the previous, e.g., the initial, administration of the angiogenesis inhibitor, e.g., a VEGF pathway inhibitor, e.g., a VEGF pathway inhibitor described herein. In some embodiments, the angiogenesis inhibitor, e.g., a VEGF pathway inhibitor, e.g., a VEGF pathway inhibitor described herein, can be administered, independently, between 17, 18, 19, 20, 21, 22, 23, 24, 25, 26, 27, 28, 29, 30, or 31 days, e.g., 21 days, after the previous, e.g., the initial, administration of the angiogenesis inhibitor, e.g., a VEGF pathway inhibitor, e.g., a VEGF pathway inhibitor described herein. After administration of the angiogenesis inhibitor, e.g., a VEGF pathway inhibitor, e.g., a VEGF pathway inhibitor described herein, the CDP-topoisomerase inhibitor conjugate, particle or composition, e.g., the CRLX101, can be administered.

In one embodiment, the CRLX101 can be administered at a dose described herein. In one embodiment, one or more subsequent administrations of the CDP-topoisomerase inhibitor conjugate, particle or composition, e.g., CRLX101, can be administered, independently, between 9, 10, 11, 12, 13, 14, 15, 16, or 17 days, e.g., 14 days, after the previous, e.g., the initial, administration of the CRLX101.

In one embodiment, one or more subsequent administrations of the CDP-topoisomerase inhibitor conjugate, particle or composition, e.g., CRLX101, can be administered, independently, between 17, 18, 19, 20, 21, 22, 23, 24, 25, 26, 27, 28, 29, 30, or 31 days, e.g., 21 days, after the previous, e.g., the initial, administration of the CRLX101. In one embodiment, the CDP-topoisomerase inhibitor conjugate, particle or composition, e.g., CRLX101, is administered 1 hour, 2 hours, 3 hours, 5 hours, 10 hours, 15 hours, 18 hours, 21 hours, or 1, 2, 3, 4, 5, 6, 7, 8, 9 or 10 days after the administration of the angiogenesis inhibitor. In one embodiment, the initial administration of the CDP-topoisomerase inhibitor conjugate, particle or composition, e.g., CRLX101, is administered on the same day, or within 1, 2, 3, 4, 5 days from the one or more subsequent doses of the angiogenesis inhibitor.

In an embodiment, the dosage of at least 2, 3, 4, 5, 6, 7, 8, 9, 10, 12, 15 or 20 administrations of the CDP-topoisomerase inhibitor conjugate, particle or composition and/or the angiogenesis inhibitor is the same.

In an embodiment, each subsequent administration of the CDP-topoisomerase inhibitor conjugate, particle or composition and/or the angiogenesis inhibitor is administered 12-16, e.g., 14, days after the previous administration.

In an embodiment, at least 2, 3, 4, 5, 6, 7, 8, 9, 10, 12, 15, 20, 50 or 100 administrations of the CDP-topoisomerase inhibitor conjugate, particle or composition and/or the angiogenesis inhibitor are administered to the subject.

In an embodiment, the conjugate includes a topoisomerase I inhibitor and/or a topoisomerase II inhibitor. In an embodiment, the conjugate includes a topoisomerase I inhibitor or combination of topoisomerase I inhibitors, e.g., camptothecin, irinotecan, SN-38, topotecan, lamellarin D and derivatives thereof. In an embodiment, the conjugate includes a topoisomerase II inhibitor or a combination of topoisomerase II inhibitors, e.g., etoposide, tenoposide, doxorubicin and derivatives thereof. In one embodiment, the conjugate includes a combination of one or more topoisomerase I inhibitors and one or more topoisomerase II inhibitors. In an embodiment, the CDP-topoisomerase inhibitor conjugate is a CDP-camptothecin or camptothecin derivate conjugate, e.g., a CDP-camptothecin or camptothecin derivative conjugate described herein, e.g., CRLX101.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases the levels of HIF-1α, HIF-2α, or both in the subject having the proliferative disorder, e.g., cancer. In some embodiments, the cancer is colon cancer. In some embodiments, the levels of HIF-1α, HIF-2α, or both are compared to a reference standard, e.g., levels of HIF-1α, HIF-2α, or both in a healthy subject that does not have cancer. In one embodiment, the method includes selecting a subject having increased levels of HIF-1α, HIF-2α, or both (e.g., as compared to a reference standard) for treatment with the conjugate, particle or composition.

In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the conjugate, particle or composition. In one embodiment, the method includes selecting a subject having or at risk of developing a metastases. In one embodiment, the method comprises administering the conjugate, particle or composition in combination with an agent or therapy that increases levels of HIF-1α, HIF-2α, or both. In one embodiment, the subject has or is at risk of developing increased HIF-1α levels, e.g., as compared to a reference standard, e.g., HIF-1α levels in a healthy subject that does not have cancer).

In one embodiment, the method comprises administering the conjugate, particle or composition in combination with an agent that increases HIF-1α levels. In another embodiment, the subject has or is at risk of developing increased HIF-2α levels, e.g., as compared to a reference standard, e.g., HIF-2α levels in a healthy subject that does not have cancer). In one embodiment, the method further comprises selecting a subject having or at risk of developing increased levels of HIF-1α, HIF-2α, or both.

In one embodiment, the cancer is a cancer described herein, and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In an embodiment, the cancer is a cancer described herein. For example, the cancer can be a cancer of the bladder (including accelerated and metastatic bladder cancer), breast (e.g., estrogen receptor positive breast cancer, estrogen receptor negative breast cancer, HER-2 positive breast cancer, HER-2 negative breast cancer, triple negative breast cancer, inflammatory breast cancer), colon (including colorectal cancer), kidney (e.g., renal cell carcinoma (e.g., papillary renal cell carcinoma, clear cell carcinoma, chromphobic carcinoma)), liver, lung (including small cell lung cancer and non-small cell lung cancer (including adenocarcinoma, squamous cell carcinoma, bronchoalveolar carcinoma and large cell carcinoma)), genitourinary tract, e.g., ovary (including fallopian, endometrial and peritoneal cancers), cervix, prostate and testes, lymphatic system, rectum, larynx, pancreas (including exocrine pancreatic carcinoma), stomach (e.g., gastroesophageal, upper gastric or lower gastric cancer), gastrointestinal cancer (e.g., anal cancer or bile duct cancer (e.g., Klatskin tumor)), gall bladder, thyroid, lymphoma (e.g., Burkitt's, Hodgkin's or non-Hodgkin's lymphoma), leukemia (e.g., acute myeloid leukemia), Ewing's sarcoma, nasoesophageal cancer, nasopharyngeal cancer, neural and glial cell cancers (e.g., glioblastoma multiforme), and head and neck.

Preferred cancers include breast cancer (e.g., metastatic or locally advanced breast cancer), prostate cancer (e.g., hormone refractory prostate cancer), renal cell carcinoma, lung cancer (e.g., small cell lung cancer and non-small cell lung cancer (including adenocarcinoma, squamous cell carcinoma, bronchoalveolar carcinoma and large cell carcinoma)), pancreatic cancer, gastric cancer (e.g., gastroesophageal, upper gastric or lower gastric cancer), colorectal cancer, squamous cell cancer of the head and neck, ovarian cancer (e.g., advanced ovarian cancer, platinum-based agent resistant or relapsed ovarian cancer), lymphoma (e.g., Burkitt's, Hodgkin's or non-Hodgkin's lymphoma), leukemia (e.g., acute myeloid leukemia) and gastrointestinal cancer.

In some embodiments, the cancer is colon cancer.

In some embodiments, the cancer is metastatic colorectal cancer (mCRC) that is resistant to or has progressed following an oxaliplatin-containing regimen.

In some embodiments, the cancer is head and neck cancer.

In some embodiments, the cancer is renal cell carcinoma.

In some embodiments, the cancer is ovarian cancer.

In some embodiments, the cancer is rectal cancer (e.g., locally advanced or metastatic rectal cancer).

In one embodiment, the cancer is, e.g., lung cancer (e.g., small cell lung cancer or non small cell lung cancer), kidney cancer (e.g., renal cell carcinoma (e.g., papillary, clear cell or chromophobic carcinoma), colorectal cancer (e.g., metastatic colorectal cancer) or glioblastoma. In one embodiment, the angiogenesis inhibitor is aflibercept and the cancer is selected from colorectal (e.g., metastatic colorectal), glioblastoma, ovarian cancer, kidney cancer (e.g., renal cell carcinoma), lung cancer (e.g., small cell lung cancer or non small cell lung cancer), pancreatic cancer, melanoma and lymphoma (e.g., non-Hodgkin's B cell lymphoma).

In an embodiment, the cancer has been sensitized to a topoisomerase inhibitor, e.g., the subject has received radiation and/or the subject has received a phosphatase inhibitor (e.g., okadiac acid) prior to the administration of the CDP-topoisomerase inhibitor conjugate, particle or composition. In one embodiment, the cancer is sensitized to topoisomerase inhibitors, e.g., the subject receives radiation in combination with the administration of the CDP-topoisomerase inhibitor conjugate, particle or composition and/or the subject is administered a phosphatase inhibitor (e.g., okadiac acid) in combination with the administration of the CDP-topoisomerase inhibitor conjugate, particle or composition. In one embodiment, the cancer is sensitized or has been sensitized to topoisomerase inhibitors and the cancer is a glial cell cancer (e.g., glioblastoma multiforme) or head and neck cancer.

In one embodiment, the subject has not been administered a CDP-topoisomerase inhibitor conjugate, particle or composition, e.g., a CDP-camptothecin or camptothecin derivative conjugate, particle or composition, a CDP-camptothecin or camptothecin derivative conjugate, particle or composition described herein, e.g., CRLX101, prior to the initial administration.

In an embodiment, the CDP-topoisomerase inhibitor conjugate, particle or composition and angiogenesis inhibitor are administered as a first line treatment for the cancer.

In an embodiment, the CDP-topoisomerase inhibitor conjugate, particle or composition and angiogenesis inhibitor are administered as a second, third or fourth line treatment for the cancer. In an embodiment, the cancer is sensitive to one or more chemotherapeutic agents, e.g., a platinum based agent, a taxane, an alkylating agent, an anthracycline (e.g., doxorubicin (e.g., liposomal doxorubicin)), an antimetabolite and/or a vinca alkaloid.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In an embodiment, the method further comprises administering to the subject a treatment that reduces one or more side effect associated with administration of a CDP-camptothecin or camptothecin derivative conjugate, particle or composition, e.g., a treatment described herein.

In one aspect, the disclosure features, a method of treating ovarian cancer (e.g., epithelial carcinoma, fallopian tube cancer, germ cell cancer (e.g., a teratoma), sex cord-stromal tumor (e.g., estrogen-producing granulose cell tumor, virilizing Sertoli-Leydig tumor, arrhenoblastoma)), e.g., locally advanced or metastatic ovarian cancer, in a subject, e.g., a human subject. The method comprises administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with an angiogenesis inhibitor (e.g., an angiogenesis inhibitor described herein).

In one embodiment, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered prior to surgery, after surgery or before and after surgery to remove the cancer, e.g., to remove a primary tumor and/or a metastases.

In one embodiment, the angiogenesis inhibitor is an inhibitor of the VEGF pathway, e.g., a VEGF inhibitor, e.g., a small molecule inhibitor, a protein, e.g., fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, pazopanib, brivanib or sunitinib, or an antibody against VEGF receptor). In one embodiment, the angiogenesis inhibitor, e.g., The VEGF inhibitor (e.g., aflibercept or bevacizumab) is administered at a dose and/or dosing schedule described herein. In one embodiment, the conjugate, particle or composition is administered at a dose and/or dosing schedule described herein. In one embodiment, when the CDP-topoisomerase inhibitor conjugate, particle or composition is administered in combination with an additional chemotherapeutic agent, the dose at which the CDP-topoisomerase inhibitor conjugate, particle or composition is administered is 1%, 3%, 5%, 10%, 15%, 20%, 25%, 30% less than a dose described herein.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having ovarian cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the ovarian cancer is, e.g., epithelial carcinoma, fallopian tube cancer, germ cell cancer (e.g., a teratoma), sex cord-stromal tumor (e.g., estrogen-producing granulose cell tumor, virilizing Sertoli-Leydig tumor, arrhenoblastoma), e.g., locally advanced or metastatic ovarian cancer, and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In one embodiment, the conjugate, particle or composition is administered in combination with a treatment that reduces one or more side effect associated with the administration of a CDP-topoisomerase inhibitor conjugate, particle or composition, e.g., a treatment described herein.

In another aspect the disclosure features a method of treating colorectal cancer (e.g., colon, small intestine, rectum and/or appendix cancer), e.g., locally advanced or metastatic colorectal cancer (e.g., locally advanced or metastatic rectal cancer), in a subject, e.g., a human subject. In some embodiments, the cancer is metastatic colorectal cancer (mCRC) that is resistant to or has progressed following treatment with an oxaliplatin-containing regimen. The method comprises administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with a second chemotherapeutic agent, e.g., the angiogenesis inhibitor, e.g., a VEGF pathway inhibitor, e.g., a VEGF pathway inhibitor described herein. In one embodiment, the CDP-topoisomerase inhibitor conjugate, particle or composition, e.g., CRLX101, and/or the angiogenesis inhibitor is administered at a dose and/or dosing schedule described herein.

In one embodiment, the VEGF pathway inhibitor is bevacizumab, and the bevacizumab is administered at a dose described herein. In one embodiment, the VEGF pathway inhibitor is aflibercept, and the aflibercept is administered at a dose described herein. In one embodiment, the cancer is refractory, relapsed or resistant to an antimetabolite and/or a platinum-based agent.

In one embodiment, the subject has increased EGFR expression levels and/or has one or more mutations in the EGFR gene, e.g., the subject has one or more of the following mutations: codon 719 of the EGFR gene (e.g., a missense mutation that results in a glycine to cysteine, alanine or serine substitution at codon 719 of the EGFR gene), codon 746 of the EGFR gene (e.g., a deletion of one or more nucleic acids of codon 746 of the EGFR gene), codon 747 of the EGFR gene (e.g., a deletion of one or more nucleic acids of codon 747 of the EGFR gene), codon 748 of the EGFR gene (e.g., a deletion of one or more nucleic acids of codon 748 of the EGFR gene), codon 749 of the EGFR gene (e.g., a deletion of one or more nucleic acids of codon 749 of the EGFR gene), codon 750 of the EGFR gene (e.g., a deletion of one or more nucleic acids of codon 750 of the EGFR gene), codon 790 of the EGFR gene (e.g. a missense mutation that results in a threonine to methionine substitution at codon 790 of the EGFR gene), codon 858 of the EGFR gene (e.g., a missense mutation that results in a leucine to arginine substitution at codon 858 of the EGFR gene), a deletion in exon 19 of the EGFR gene, and an insert mutation at exon 20 of the EGFR gene.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having colorectal cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the colorectal cancer is, e.g., colon, small intestine, rectum and/or appendix cancer), e.g., locally advanced or metastatic colorectal cancer (e.g., locally advanced or metastatic rectal cancer), and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered prior to surgery, after surgery or before and after surgery to remove the cancer, e.g., to remove the primary tumor and/or a metastases.

In one embodiment, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered in combination with one or more radiation treatments, e.g., multiple radiation treatments, e.g., pelvic radiation treatments. In one embodiment, the method comprises multiple radiation treatments and an initial radiation treatment, e.g., pelvic radiation treatment, is administered with the administration of said CDP-topoisomerase inhibitor conjugate, particle or composition, e.g., with the initial administration, of said CDP-topoisomerase inhibitor conjugate, particle or composition.

In one embodiment, the conjugate, particle or composition is administered in combination with a VEGF pathway inhibitor, e.g., bevacizumab, and an antimetabolite, e.g., an antifolate (e.g., pemetrexed, floruridine, raltitrexed) or pyrimidine analogue (e.g., capecitabine, 5FU, cytrarabine, gemcitabine). In one embodiment, the conjugate, particle or composition is administered with a VEGF pathway inhibitor, e.g., bevacizumab, an antimetabolite, e.g., a pyrimidine analogue (e.g., 5FU), and folinic acid (leucovorin). In another embodiment, the conjugate, particle or composition is administered with a VEGF pathway inhibitor, e.g., bevacizumab, an antimetabolite, e.g., a pyrimidine analogue (e.g., 5FU), folinic acid (leucovorin), and a platinum-based agent (e.g., cisplatin, carboplatin, oxaliplatin). In one embodiment, the cancer is refractory, relapsed or resistant to an antimetabolite and/or a platinum-based agent.

In another embodiment, a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered in combination with a VEGF pathway inhibitor, e.g., bevacizumab or aflibercept, and an antimetabolite wherein the antimetabolite is a pyrimidine analogue, e.g., capecitabine. In one embodiment, the conjugate, particle or composition is further administered in combination with a platinum-based agent (e.g., cisplatin, carboplatin, oxaliplatin). For example, in one embodiment, the conjugate, particle or composition is administered with the following combination: a VEGF pathway inhibitor, e.g., a VEGF inhibitor (e.g., bevacizumab or aflibercept) or a VEGF receptor inhibitor, a pyrimidine analogue (e.g., capecitabine), and a platinum-based agent (e.g., oxaliplatin); or a VEGF pathway inhibitor (e.g., bevacizumab or aflibercept) and a pyrimidine analogue (e.g., capecitabine).

In one embodiment, the conjugate, particle or composition is administered in combination with a treatment that reduces one or more side effect associated with the administration of a CDP-topoisomerase inhibitor conjugate, particle or composition, e.g., a treatment described herein.

In one aspect, the disclosure features a method of treating lung cancer (e.g., small cell lung cancer or non-small cell lung cancer (e.g., adenocarcinoma, squamous cell carcinoma, bronchoalveolar carcinoma and large cell carcinoma)), e.g., locally advanced or metastatic lung cancer, in a subject, e.g., a human subject. The method comprises administering a CDP-topoisomerase inhibitor conjugate, particle or composition, e.g., a CDP-topoisomerase I or II inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with a second chemotherapeutic agent, e.g., an angiogenesis inhibitor, e.g., a VEGF pathway inhibitor, e.g., a VEGF pathway inhibitor described herein. In one embodiment, the CDP-topoisomerase inhibitor conjugate, particle or composition, e.g., CRLX101, and/or the angiogenesis inhibitor is administered at a dose and/or dosing schedule described herein.

In one embodiment, the VEGF pathway inhibitor is bevacizumab, and the bevacizumab is administered at a dose described herein. In one embodiment, the VEGF pathway inhibitor is aflibercept, and the aflibercept is administered at a dose described herein.

In one embodiment, the method comprises selecting a subject that has squamous cell lung cancer for treatment.

In one embodiment, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered prior to surgery, after surgery or before and after surgery to remove the cancer, e.g., to remove a primary tumor and/or a metastases.

In one embodiment, the method includes selecting a subject who has lung cancer and who has increased KRAS and/or ST expression levels, e.g., as compared to a reference standard, and/or has a mutation in a KRAS and/or ST gene; and

administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with the second chemotherapeutic agent, to the subject in an amount effective to treat the cancer, to thereby treat the cancer.

In one embodiment, the subject has increased KRAS and/or ST expression levels, e.g., as compared to a reference standard, and/or has a mutation in a KRAS and/or ST gene. In one embodiment, the subject has a mutation at one or more of: codon 12 of the KRAS gene (e.g., a G to T transversion, a G to C transversion, or a G to S transversion), codon 13 of the KRAS gene, codon 61 of the KRAS gene. In one embodiment, the subject has non small cell lung cancer associated with mucinous broncholoalveolar cells or goblet cells.

In one embodiment, the method includes selecting a subject who has lung cancer and who has a mutation in an EGFR gene; and

administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with the second chemotherapeutic agent, to the subject in an amount effective to treat the cancer, to thereby treat the cancer.

In one embodiment, the subject has lung cancer that is resistant, relapsed or refractory to an EGF pathway inhibitor, e.g., an EGF receptor inhibitor (e.g., erlotinib)

In one embodiment, the subject has one or more of the following mutations: codon 719 of the EGFR gene (e.g., a missense mutation that results in a glycine to cysteine, alanine or serine substitution at codon 719 of the EGFR gene), codon 746 of the EGFR gene (e.g., a deletion of one or more nucleic acids of codon 746 of the EGFR gene), codon 747 of the EGFR gene (e.g., a deletion of one or more nucleic acids of codon 747 of the EGFR gene), codon 748 of the EGFR gene (e.g., a deletion of one or more nucleic acids of codon 748 of the EGFR gene), codon 749 of the EGFR gene (e.g., a deletion of one or more nucleic acids of codon 749 of the EGFR gene), codon 750 of the EGFR gene (e.g., a deletion of one or more nucleic acids of codon 750 of the EGFR gene), codon 790 of the EGFR gene (e.g. a missense mutation that results in a threonine to methionine substitution at codon 790 of the EGFR gene), codon 858 of the EGFR gene (e.g., a missense mutation that results in a leucine to arginine substitution at codon 858 of the EGFR gene), a deletion in exon 19 of the EGFR gene, and an insert mutation at exon 20 of the EGFR gene.

In one embodiment, the subject has a mutation in the EGFR gene and has a mutation in the KRAS gene and/or overexpresses KRAS, e.g., as compared to a reference standard (e.g. codon 12 of the KRAS gene (e.g. a missense mutation that results in a glycine to cysteine substitution at codon 12 of the KRAS gene; a missense mutation that results in a glycine to serine at codon 12 of the KRAS gene).

In one embodiment, the method includes selecting a subject who has lung cancer and who does not have a mutation in an EGFR gene; and

administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with the second chemotherapeutic agent, to the subject in an amount effective to treat the cancer, to thereby treat the cancer.

In one embodiment, the method includes selecting a subject who has squamous cell lung cancer; and

administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with the second chemotherapeutic agent, to the subject in an amount effective to treat the cancer, to thereby treat the cancer.

In one embodiment, the subject does not have one or more of the following mutations: codon 719 of the EGFR gene (e.g., a missense mutation that results in a glycine to cysteine, alanine or serine substitution at codon 719 of the EGFR gene), codon 746 of the EGFR gene (e.g., a deletion of one or more nucleic acids of codon 746 of the EGFR gene), codon 747 of the EGFR gene (e.g., a deletion of one or more nucleic acids of codon 747 of the EGFR gene), codon 748 of the EGFR gene (e.g., a deletion of one or more nucleic acids of codon 748 of the EGFR gene), codon 749 of the EGFR gene (e.g., a deletion of one or more nucleic acids of codon 749 of the EGFR gene), codon 750 of the EGFR gene (e.g., a deletion of one or more nucleic acids of codon 750 of the EGFR gene), codon 790 of the EGFR gene (e.g. a missense mutation that results in a threonine to methionine substitution at codon 790 of the EGFR gene), codon 858 of the EGFR gene (e.g., a missense mutation that results in a leucine to arginine substitution at codon 858 of the EGFR gene), a deletion in exon 19 of the EGFR gene, and an insert mutation at exon 20 of the EGFR gene.

In one embodiment, the subject has a mutation in the KRAS gene and/or overexpresses KRAS, e.g., as compared to a reference standard, (e.g. codon 12 of the KRAS gene (e.g. a missense mutation that results in a glycine to cysteine substitution at codon 12 of the KRAS gene; a missense mutation that results in a glycine to serine substitution at codon 12 of the KRAS gene), and does not have a mutation in the EGFR gene.

In one embodiment, the subject is refractory, relapsed or resistant to one or more chemotherapeutic agents, e.g., a taxane (e.g., docetaxel), a platinum-based agent (e.g., carboplatin, cisplatin, oxaliplatin) and/or an EGF pathway inhibitor, e.g., an EGF inhibitor or and EGFR inhibitor, e.g., erlotinib.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having lung cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the lung cancer is, e.g., small cell lung cancer or non-small cell lung cancer (e.g., adenocarcinoma, squamous cell carcinoma, bronchoalveolar carcinoma and large cell carcinoma)), e.g., locally advanced or metastatic lung cancer, and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the conjugate, particle or composition is administered at a dose and/or dosing schedule described herein.

In one embodiment, the angiogenesis inhibitor is a VEGF pathway inhibitor, e.g., a VEGF inhibitor, e.g., a small molecule inhibitor, a protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, pazopanib, brivanib or sunitinib, or an antibody against VEGF receptor, is administered at a dose and/or dosing schedule described herein.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In one embodiment, the conjugate, particle or composition is administered in combination with a treatment that reduces one or more side effect associated with the administration of a CDP-topoisomerase inhibitor conjugate, particle or composition, e.g., a treatment described herein.

In one aspect, the disclosure features a method of treating breast cancer (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer)), e.g., locally advanced or metastatic breast cancer, in a subject, e.g., a human subject. The method comprises administering a CDP-topoisomerase inhibitor conjugate, particle or composition, e.g., a topoisomerase I or II inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with a second chemotherapeutic agent, e.g., an angiogenesis inhibitor, e.g., a VEGF pathway inhibitor, e.g., a VEGF pathway inhibitor described herein. In one embodiment, the CDP-topoisomerase inhibitor conjugate, particle or composition, e.g., CRLX101, and/or the angiogenesis inhibitor is administered at a dose and/or dosing schedule described herein.

In one embodiment, the VEGF pathway inhibitor is bevacizumab, and the bevacizumab is administered at a dose described herein. In one embodiment, the VEGF pathway inhibitor is aflibercept, and the aflibercept is administered at a dose described herein.

In one embodiment, the subject has increased EGFR expression levels and/or has one or more mutations in the EGFR gene, e.g., the subject has one or more of the following mutations: codon 719 of the EGFR gene (e.g., a missense mutation that results in a glycine to cysteine, alanine or serine substitution at codon 719 of the EGFR gene), codon 746 of the EGFR gene (e.g., a deletion of one or more nucleic acids of codon 746 of the EGFR gene), codon 747 of the EGFR gene (e.g., a deletion of one or more nucleic acids of codon 747 of the EGFR gene), codon 748 of the EGFR gene (e.g., a deletion of one or more nucleic acids of codon 748 of the EGFR gene), codon 749 of the EGFR gene (e.g., a deletion of one or more nucleic acids of codon 749 of the EGFR gene), codon 750 of the EGFR gene (e.g., a deletion of one or more nucleic acids of codon 750 of the EGFR gene), codon 790 of the EGFR gene (e.g. a missense mutation that results in a threonine to methionine substitution at codon 790 of the EGFR gene), codon 858 of the EGFR gene (e.g., a missense mutation that results in a leucine to arginine substitution at codon 858 of the EGFR gene), a deletion in exon 19 of the EGFR gene, and an insert mutation at exon 20 of the EGFR gene.

In one embodiment, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered prior to surgery, after surgery or before and after surgery to remove the cancer, e.g., to remove a primary tumor and/or a metastases.

In some embodiments, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered in combination with a vascular endothelial growth factor (VEGF) pathway inhibitor, e.g., a VEGF pathway inhibitor described herein, e.g., a VEGF inhibitor (e.g., bevacizumab or aflibercept) or VEGF receptor inhibitor (e.g., sorafenib, sunitinib, pazopanib, brivanib, CP-547632 and AZD2171), and further comprises administering a taxane (e.g., paclitaxel, docetaxel, larotaxel, cabazitaxel) and/or further comprises administering a poly ADP-ribose polymerase (PARP) inhibitor (e.g., BSI 201, Olaparib (AZD-2281), ABT-888, AG014699, CEP 9722, MK 4827, KU-0059436 (AZD2281), LT-673, 3-aminobenzamide).

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having breast cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the breast cancer is, e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer), e.g., locally advanced or metastatic breast cancer, and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the conjugate, particle or composition is administered in combination with an angiogenesis inhibitor (e.g., an angiogenesis inhibitor described herein such as an inhibitor of the VEGF pathway), at a dose and/or dosing schedule described herein.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In one embodiment, the conjugate, particle or composition is administered in combination with a treatment that reduces one or more side effect associated with the administration of a CDP-topoisomerase inhibitor conjugate, particle or composition, e.g., a treatment described herein.

In one aspect, the disclosure features a method of treating gastric cancer (e.g., gastric adenocarcinoma (e.g., intestinal or diffuse), gastric lymphoma (e.g., MALT lymphoma), carcinoid stromal tumor), e.g., locally advanced or metastatic gastric cancer, in a subject, e.g., a human subject. The method comprises administering a CDP-topoisomerase inhibitor conjugate, particle or composition, e.g., a CDP-topoisomerase I or II inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, in combination with a second chemotherapeutic agent, e.g., an angiogenesis inhibitor, e.g., a VEGF pathway inhibitor, e.g., a VEGF pathway inhibitor described herein. In one embodiment, the CDP-topoisomerase inhibitor conjugate, particle or composition, e.g., CRLX101, and/or the angiogenesis inhibitor is administered at a dose and/or dosing schedule described herein.

In one embodiment, the VEGF pathway inhibitor is bevacizumab, and the bevacizumab is administered at a dose described herein. In one embodiment, the VEGF pathway inhibitor is aflibercept, and the aflibercept is administered at a dose described herein.

In one embodiment, the gastric cancer is gastroesophageal junction adenocarcinoma.

In one embodiment, the CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, is administered prior to surgery, after surgery or before and after surgery to remove the cancer, e.g., to remove a primary tumor and/or a metastases.

In one embodiment, the angiogenesis inhibitor is an inhibitor of the VEGF pathway, e.g., a VEGF inhibitor, e.g., a small molecule inhibitor, protein, e.g., fusion protein (e.g., aflibercept) or an antibody against VEGF, e.g., bevacizumab; or a VEGF receptor inhibitor, e.g., a VEGF receptor 2 inhibitor, e.g., a small molecule inhibitor, e.g., sorafenib, pazopanib, brivanib or sunitinib, or an antibody against VEGF receptor 2; or a VEGF receptor 1 inhibitor, e.g., a small molecule inhibitor, or an antibody against VEGF receptor 1), and, e.g., is administered at a dose and/or dosing schedule described herein.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having gastric cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the gastric cancer is, e.g., gastric adenocarcinoma (e.g., intestinal or diffuse), gastric lymphoma (e.g., MALT lymphoma), carcinoid stromal tumor), e.g., locally advanced or metastatic gastric cancer, in a subject, and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In one embodiment, the conjugate, particle or composition is administered in combination with a treatment that reduces one or more side effect associated with the administration of a CDP-topoisomerase inhibitor conjugate, particle or composition, e.g., a treatment described herein.

In one aspect, the invention features, a method of treating pancreatic cancer in a subject, the method comprising, administering a CDP-topoisomerase inhibitor conjugate, particle or composition to the subject in combination with a second chemotherapeutic agent, e.g., an angiogenesis inhibitor, e.g., a VEGF pathway inhibitor, e.g., a VEGF pathway inhibitor described herein. In one embodiment, the CDP-topoisomerase inhibitor conjugate, particle or composition, e.g., CRLX101, and/or the angiogenesis inhibitor is administered at a dose and/or dosing schedule described herein.

In one embodiment, the VEGF pathway inhibitor is bevacizumab, and the bevacizumab is administered at a dose described herein. In one embodiment, the VEGF pathway inhibitor is aflibercept, and the aflibercept is administered at a dose described herein.

In one embodiment, the subject has increased EGFR expression levels and/or has one or more mutations in the EGFR gene, e.g., the subject has one or more of the following mutations: codon 719 of the EGFR gene (e.g., a missense mutation that results in a glycine to cysteine, alanine or serine substitution at codon 719 of the EGFR gene), codon 746 of the EGFR gene (e.g., a deletion of one or more nucleic acids of codon 746 of the EGFR gene), codon 747 of the EGFR gene (e.g., a deletion of one or more nucleic acids of codon 747 of the EGFR gene), codon 748 of the EGFR gene (e.g., a deletion of one or more nucleic acids of codon 748 of the EGFR gene), codon 749 of the EGFR gene (e.g., a deletion of one or more nucleic acids of codon 749 of the EGFR gene), codon 750 of the EGFR gene (e.g., a deletion of one or more nucleic acids of codon 750 of the EGFR gene), codon 790 of the EGFR gene (e.g. a missense mutation that results in a threonine to methionine substitution at codon 790 of the EGFR gene), codon 858 of the EGFR gene (e.g., a missense mutation that results in a leucine to arginine substitution at codon 858 of the EGFR gene), a deletion in exon 19 of the EGFR gene, and an insert mutation at exon 20 of the EGFR gene.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having pancreatic cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the cancer is pancreatic cancer, and the subject selected for treatment overexpresses carbonic anhydrase IX (CAIX). In one embodiment, the method further comprises acquiring CAIX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In one embodiment, the conjugate, particle or composition is administered in combination with a treatment that reduces one or more side effect associated with the administration of a CDP-topoisomerase inhibitor conjugate, particle or composition, e.g., a treatment described herein.

In one aspect, the invention features, a method of treating renal cell carcinoma in a subject, the method comprising, administering a CDP-topoisomerase inhibitor conjugate, particle or composition to the subject in combination with a second chemotherapeutic agent, e.g., an angiogenesis inhibitor, e.g., a VEGF pathway inhibitor, e.g., a VEGF pathway inhibitor described herein. In one embodiment, the CDP-topoisomerase inhibitor conjugate, particle or composition, e.g., CRLX101, and/or the angiogenesis inhibitor is administered at a dose and/or dosing schedule described herein.

In one embodiment, the VEGF pathway inhibitor is bevacizumab, and the bevacizumab is administered at a dose described herein. In one embodiment, the VEGF pathway inhibitor is aflibercept, and the aflibercept is administered at a dose described herein.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having renal cell carcinoma. In some embodiments, HIF-1α levels, HIF-2α levels, or both are compared to a reference standard, e.g., HIF-la levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent or therapy that increases HIF-1α levels, HIF-2α levels, or both.

In one embodiment, the renal cell carcinoma is, e.g., clear cell renal cell carcinoma, and the subject selected for treatment overexpresses carbonic anhydrase IX (CALX). In one embodiment, the method further comprises acquiring CALX expression levels in the subject, e.g., prior to, concurrent with or after administration of the CDP-topoisomerase inhibitor conjugate, particle or composition. In one embodiment, CAIX expression levels are determined prior to treatment and, e.g., every two, three, four or five weeks after the initial administration of the CDP-topoisomerase inhibitor conjugate, particle or composition.

In one embodiment, CAIX expression levels are determined after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101, e.g., 1, 2, 3, 4, 5, 6, 7, 8, 9, or 10 weeks after the last administration of the CDP-topoisomerase inhibitor conjugate, particle or composition, e.g., CDP-camptothecin conjugate, particle or composition or camptothecin derivative conjugate, particle or composition, e.g., CDP-camptothecin conjugate, particle or composition or camptothecin derivative conjugate, particle or composition described herein, e.g., CRLX101.

In one embodiment, the method further comprises administering one or more chemotherapeutic agents, e.g., one or more chemotherapeutic agent described herein, in combination with the CDP-topoisomerase inhibitor conjugate, particle or composition and the angiogenesis inhibitor. For example, in one embodiment, the method comprises administering CDP-topoisomerase inhibitor conjugate, particle or composition in combination with the angiogenesis inhibitor and a taxane (e.g., docetaxel, paclitaxel, larotaxel cabazitaxel).

In one embodiment, the conjugate, particle or composition is administered in combination with a treatment that reduces one or more side effect associated with the administration of a CDP-topoisomerase inhibitor conjugate, particle or composition, e.g., a treatment described herein.

### CAIX

In yet another aspect, the disclosure features a method of treating a cancer in a subject, e.g., a human subject, the method comprising:

selecting a subject who has a cancer, e.g., a cancer associated with increased CAIX expression levels, e.g., as compared to a reference standard (e.g., CAIX levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, comprises:

providing an initial administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to said subject at a dosage of 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², (wherein said dosage is expressed in mg of drug, as opposed to mg of conjugate); and

providing one or more subsequent administrations of said CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dosage of 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², wherein each subsequent administration is provided, independently, between 12, 13, 14, 15 or 16 days, after the previous, e.g., the initial, administration.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating CAIX expression levels in the sample as compared to a reference standard (e.g., CAIX levels in a healthy subject or CAIX levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, comprises:

providing an initial administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to said subject at a dosage of 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², (wherein said dosage is expressed in mg of drug, as opposed to mg of conjugate); and

providing one or more subsequent administrations of said CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dosage of 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², wherein each subsequent administration is provided, independently, between 12, 13, 14, 15 or 16 days, after the previous, e.g., the initial, administration.

In one embodiment, the method further comprises determining the CAIX expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of CAIX expression can be determined by the amount of CAIX nucleic acid (e.g., mRNA) in the sample, e.g., by any suitable method, e.g., northern blotting, RT-PCR, or the use of biochips.

In one embodiment, the level of CAIX expression can be determined by the amount of CAIX protein, e.g., by any suitable method.

In one embodiment, the level of CAIX protein expression can be determined by a ligand binding assay (LBA).

In one embodiment, the level of CAIX protein expression can be determined by protein immunostaining, e.g., immunohistochemistry, or e.g., immunofluorescence assay.

In one embodiment, the level of CAIX protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased CAIX expression levels, e.g., as compared to a reference standard (e.g., CAIX levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring CAIX expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the CAIX expression levels in the subject are decreased as compared, e.g., to the CAIX expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial CAIX expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, comprises:

providing an initial administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to said subject at a dosage of 6 m g/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², (wherein said dosage is expressed in mg of drug, as opposed to mg of conjugate); and

providing one or more subsequent administrations of said CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dosage of 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², wherein each subsequent administration is provided, independently, between 12, 13, 14, 15 or 16 days, after the previous, e.g., the initial, administration.

In yet another aspect, the disclosure features, a method of treating a cancer, in a subject, e.g., a human subject, the method comprising:

selecting a subject who has cancer, e.g., a cancer associated with increased CAIX expression levels, e.g., as compared to a reference standard (e.g., CAIX levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring CAIX expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the CAIX expression levels in the subject are the same or increased, e.g., as compared to the CAIX expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial CAIX expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased CAIX expression levels, e.g., as compared to a reference standard (e.g., CAIX levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating CAIX expression levels in the biological sample, e.g., as compared to the reference standard or as compared to CAIX expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the CAIX expression levels decrease as compared to the CAIX expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the CAIX expression levels are the same or increased as compared to the CAIX expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of CAIX expression can be determined by the amount of CAIX nucleic acid (e.g., mRNA) in the sample, e.g., by any suitable method, e.g., northern blotting, RT-PCR, or the use of biochips.

In one embodiment, the level of CAIX expression can be determined by the amount of CAIX protein, e.g., by any suitable method.

In one embodiment, the level of CAIX protein expression can be determined by a ligand binding assay (LBA).

In one embodiment, the level of CAIX protein expression can be determined by protein immunostaining, e.g., immunohistochemistry, or e.g., immunofluorescence assay.

In one embodiment, the level of CAIX protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects CAIX expression levels.

In one embodiment, the cancer is selected from ovarian cancer, rectal cancer and renal cell carcinoma.

In some embodiments, the agent that detects CAIX expression levels is a reagent used in a ligand binding assay (LBA).

In some embodiments, the agent that detects CAIX expression levels is a reagent used in a protein immunostaining assay, e.g., immunohistochemistry assay, or e.g., an immunofluorescence assay.

In some embodiments, the agent that detects CAIX expression levels is a reagent used in a western blot assay, or in an ELISA assay.

In one embodiment, the agent detects CAIX protein, e.g., an anti-CAIX antibody.

In one embodiment, the agent detects a nucleic acid encoding CAM e.g., a probe that binds to a nucleic acid encoding CAIX.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: PAI-1, VEGF, H2AX, RAD51, topo 1 or topo 2. In one embodiment, the reaction mixture includes two or more additional agents (e.g., 2, 3, 4, 5 or 6 agents) selected from an agent that detects PAI-1, an agent that detects VEGF, an agent that detects H2AX, an agent that detects RAD51, an agent that detects topo 1 and an agent that detects topo 2.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects CAIX expression levels.

In one embodiment, the cancer is selected from ovarian cancer, rectal cancer and renal cell carcinoma.

In some embodiments, the agent that detects CAIX expression levels is a reagent used in a ligand binding assay (LBA).

In some embodiments, the agent that detects CAIX expression levels is a reagent used in a protein immunostaining assay, e.g., immunohistochemistry assay, or e.g., an immunofluorescence assay.

In some embodiments, the agent that detects CAIX expression levels is a reagent used in a western blot assay, or in an ELISA assay.

In one embodiment, the agent detects CAIX protein, e.g., an anti-CAIX antibody.

In one embodiment, the agent detects a nucleic acid encoding CAM e.g., a probe that binds to a nucleic acid encoding CAIX.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

### Plasminogen Activator Inhibitor-1 (PAI-1)

In yet another aspect, the disclosure features a method of treating a cancer in a subject, e.g., a human subject, the method comprising:

selecting a subject who has a cancer, e.g., a cancer associated with increased PAI-1 expression levels, e.g., as compared to a reference standard (e.g., PAI-1 levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating PAI-1 expression levels in the sample as compared to a reference standard (e.g., PAI-1 levels in a healthy subject or PAI-1 levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the method further comprises determining the PAI-1 expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of PAI-1 expression can be determined by the amount of PAI-1 nucleic acid (e.g., mRNA) in the sample, e.g., by any suitable method, e.g., northern blotting, RT-PCR, or the use of biochips.

In one embodiment, the level of PAI-1 expression can be determined by the amount of PAI-1 protein, e.g., by any suitable method.

In one embodiment, the level of PAI-1 protein expression can be determined by a ligand binding assay (LBA).

In one embodiment, the level of PAI-1 protein expression can be determined by protein immunostaining, e.g., immunohistochemistry, or e.g., immunofluorescence assay.

In one embodiment, the level of PAI-1 protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased PAI-1 expression levels, e.g., as compared to a reference standard (e.g., PAI-1 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring PAI-1 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the PAI-1 expression levels in the subject are decreased as compared, e.g., to the PAI-1 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial PAI-1 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In yet another aspect, the disclosure features, a method of treating a cancer, in a subject, e.g., a human subject, the method comprising:

selecting a subject who has cancer, e.g., a cancer associated with increased PAI-1 expression levels, e.g., as compared to a reference standard (e.g., PAI-1 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring PAI-1 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the PAI-1 expression levels in the subject are the same or increased, e.g., as compared to the PAI-1 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial PAI-1 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased PAI-1 expression levels, e.g., as compared to a reference standard (e.g., PAI-1 levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating PAI-1 expression levels in the biological sample, e.g., as compared to the reference standard or as compared to PAI-1 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the PAI-1 expression levels decrease as compared to the PAI-1 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the PAI-1 expression levels are the same or increased as compared to the PAI-1 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of PAI-1 expression can be determined by the amount of PAI-1 nucleic acid (e.g., mRNA) in the sample, e.g., by any suitable method, e.g., northern blotting, RT-PCR, or the use of biochips.

In one embodiment, the level of PAI-1 expression can be determined by the amount of PAI-1 protein, e.g., by any suitable method.

In one embodiment, the level of PAI-1 protein expression can be determined by a ligand binding assay (LBA).

In one embodiment, the level of PAI-1 protein expression can be determined by protein immunostaining, e.g., immunohistochemistry, or e.g., immunofluorescence assay.

In one embodiment, the level of PAI-1 protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects PAI-1 expression levels.

In one embodiment, the cancer is selected from ovarian cancer, rectal cancer and renal cell carcinoma.

In some embodiments, the agent that detects PAI-1 expression levels is a reagent used in a ligand binding assay (LBA).

In some embodiments, the agent that detects PAI-1 expression levels is a reagent used in a protein immunostaining assay, e.g., immunohistochemistry assay, or e.g., an immunofluorescence assay.

In some embodiments, the agent that detects PAI-1 expression levels is a reagent used in a western blot assay, or in an ELISA assay.

In one embodiment, the agent detects PAI-1 protein, e.g., an anti-PAI-1 antibody.

In one embodiment, the agent detects a nucleic acid encoding PAI-1, e.g., a probe that binds to a nucleic acid encoding PAI-1.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: CAM VEGF, H2AX, RAD51, topo 1 or topo 2. In one embodiment, the reaction mixture includes two or more additional agents (e.g., 2, 3, 4, or 6 agents) selected from an agent that detects CAM an agent that detects VEGF, an agent that detects H2AX, an agent that detects RAD51, an agent that detects topo 1 and an agent that detects topo 2.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects PAI-1 expression levels.

In one embodiment, the cancer is selected from ovarian cancer, rectal cancer and renal cell carcinoma.

In some embodiments, the agent that detects PAI-1 expression levels is a reagent used in a ligand binding assay (LBA).

In some embodiments, the agent that detects PAI-1 expression levels is a reagent used in a protein immunostaining assay, e.g., immunohistochemistry assay, or e.g., an immunofluorescence assay.

In some embodiments, the agent that detects PAI-1 expression levels is a reagent used in a western blot assay, or in an ELISA assay.

In one embodiment, the agent detects PAI-1 protein, e.g., an anti-PAI-1 antibody.

In one embodiment, the agent detects a nucleic acid encoding PAI-1, e.g., a probe that binds to a nucleic acid encoding PAI-1.

In one embodiment, the biological sample is a biopsy sample or a sample obtained from a lesion removed from the subject, e.g., a primary or metastatic lesion. In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

### Vascular Endothelial Growth Factor (VEGF)

In yet another aspect, the disclosure features a method of treating a cancer in a subject, e.g., a human subject, the method comprising:

selecting a subject who has a cancer, e.g., a cancer associated with increased VEGF expression levels, e.g., as compared to a reference standard (e.g., VEGF levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating VEGF expression levels in the sample as compared to a reference standard (e.g., VEGF levels in a healthy subject or VEGF levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the method further comprises determining the VEGF expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of VEGF expression can be determined by the amount of VEGF nucleic acid (e.g., mRNA) in the sample, e.g., by any suitable method, e.g., northern blotting, RT-PCR, or the use of biochips.

In one embodiment, the level of VEGF expression can be determined by the amount of VEGF protein, e.g., by any suitable method.

In one embodiment, the level of VEGF protein expression can be determined by a ligand binding assay (LBA).

In one embodiment, the level of VEGF protein expression can be determined by protein immunostaining, e.g., immunohistochemistry, or e.g., immunofluorescence assay.

In one embodiment, the level of VEGF protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased VEGF expression levels, e.g., as compared to a reference standard (e.g., VEGF levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring VEGF expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the VEGF expression levels in the subject are decreased as compared, e.g., to the VEGF expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial VEGF expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In yet another aspect, the disclosure features, a method of treating a cancer, in a subject, e.g., a human subject, the method comprising:

selecting a subject who has cancer, e.g., a cancer associated with increased VEGF expression levels, e.g., as compared to a reference standard (e.g., VEGF levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring VEGF expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the VEGF expression levels in the subject are the same or increased, e.g., as compared to the VEGF expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial VEGF expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased VEGF expression levels, e.g., as compared to a reference standard (e.g., VEGF levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating VEGF expression levels in the biological sample, e.g., as compared to the reference standard or as compared to VEGF expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the VEGF expression levels decrease as compared to the VEGF expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the VEGF expression levels are the same or increased as compared to the VEGF expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of VEGF expression can be determined by the amount of VEGF nucleic acid (e.g., mRNA) in the sample, e.g., by any suitable method, e.g., northern blotting, RT-PCR, or the use of biochips.

In one embodiment, the level of VEGF expression can be determined by the amount of VEGF protein, e.g., by any suitable method.

In one embodiment, the level of VEGF protein expression can be determined by a ligand binding assay (LBA).

In one embodiment, the level of VEGF protein expression can be determined by protein immunostaining, e.g., immunohistochemistry, or e.g., immunofluorescence assay.

In one embodiment, the level of VEGF protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects VEGF expression levels.

In one embodiment, the cancer is selected from ovarian cancer, rectal cancer and renal cell carcinoma.

In some embodiments, the agent that detects VEGF expression levels is a reagent used in a ligand binding assay (LBA).

In some embodiments, the agent that detects VEGF expression levels is a reagent used in a protein immunostaining assay, e.g., immunohistochemistry assay, or e.g., an immunofluorescence assay.

In some embodiments, the agent that detects VEGF expression levels is a reagent used in a western blot assay, or in an ELISA assay.

In one embodiment, the agent detects VEGF protein, e.g., an anti-VEGF antibody.

In one embodiment, the agent detects a nucleic acid encoding VEGF, e.g., a probe that binds to a nucleic acid encoding VEGF.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: PAI-1, CAIX, H2AX, RAD51, topo 1 or topo 2. In one embodiment, the reaction mixture includes two or more additional agents (e.g., 2, 3, 4, 5 or 6 agents) selected from an agent that detects PAI-1, an agent that detects CAM an agent that detects H2AX, an agent that detects RAD51, an agent that detects topo 1 and an agent that detects topo 2.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects VEGF expression levels.

In one embodiment, the cancer is selected from ovarian cancer, rectal cancer and renal cell carcinoma.

In some embodiments, the agent that detects VEGF expression levels is a reagent used in a ligand binding assay (LBA).

In some embodiments, the agent that detects VEGF expression levels is a reagent used in a protein immunostaining assay, e.g., immunohistochemistry assay, or e.g., an immunofluorescence assay.

In some embodiments, the agent that detects VEGF expression levels is a reagent used in a western blot assay, or in an ELISA assay.

In one embodiment, the agent detects VEGF protein, e.g., an anti-VEGF antibody.

In one embodiment, the agent detects a nucleic acid encoding VEGF, e.g., a probe that binds to a nucleic acid encoding VEGF.

In one embodiment, the biological sample is a biopsy sample or a sample obtained from a lesion removed from the subject, e.g., a primary or metastatic lesion. In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

gamma-H2AX

In yet another aspect, the disclosure features a method of treating a cancer in a subject, e.g., a human subject, the method comprising:

selecting a subject who has a cancer, e.g., a cancer associated with increased gamma-H2AX expression levels, e.g., as compared to a reference standard (e.g., gamma-H2AX levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating gamma-H2AX expression levels in the sample as compared to a reference standard (e.g., gamma-H2AX levels in a healthy subject or gamma-H2AX levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the method further comprises determining the gamma-H2AX expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of gamma-H2AX expression can be determined by the amount of gamma-H2AX nucleic acid (e.g., mRNA) in the sample, e.g., by any suitable method, e.g., northern blotting, RT-PCR, or the use of biochips.

In one embodiment, the level of gamma-H2AX expression can be determined by the amount of gamma-H2AX protein, e.g., by any suitable method.

In one embodiment, the level of gamma-H2AX protein expression can be determined by a ligand binding assay (LBA).

In one embodiment, the level of gamma-H2AX protein expression can be determined by protein immunostaining, e.g., immunohistochemistry, or e.g., immunofluorescence assay.

In one embodiment, the level of gamma-H2AX protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased gamma-H2AX expression levels, e.g., as compared to a reference standard (e.g., gamma-H2AX levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring gamma-H2AX expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the gamma-H2AX expression levels in the subject are decreased as compared, e.g., to the gamma-H2AX expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial gamma-H2AX expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In yet another aspect, the disclosure features, a method of treating a cancer, in a subject, e.g., a human subject, the method comprising:

selecting a subject who has cancer, e.g., a cancer associated with increased gamma-H2AX expression levels, e.g., as compared to a reference standard (e.g., gamma-H2AX levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring gamma-H2AX expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the gamma-H2AX expression levels in the subject are the same or increased, e.g., as compared to the gamma-H2AX expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial gamma-H2AX expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased gamma-H2AX expression levels, e.g., as compared to a reference standard (e.g., gamma-H2AX levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating gamma-H2AX expression levels in the biological sample, e.g., as compared to the reference standard or as compared to gamma-H2AX expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the gamma-H2AX expression levels decrease as compared to the gamma-H2AX expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the gamma-H2AX expression levels are the same or increased as compared to the gamma-H2AX expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of gamma-H2AX expression can be determined by the amount of gamma-H2AX nucleic acid (e.g., mRNA) in the sample, e.g., by any suitable method, e.g., northern blotting, RT-PCR, or the use of biochips.

In one embodiment, the level of gamma-H2AX expression can be determined by the amount of gamma-H2AX protein, e.g., by any suitable method.

In one embodiment, the level of gamma-H2AX protein expression can be determined by a ligand binding assay (LBA).

In one embodiment, the level of gamma-H2AX protein expression can be determined by protein immunostaining, e.g., immunohistochemistry, or e.g., immunofluorescence assay.

In one embodiment, the level of gamma-H2AX protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects gamma-H2AX expression levels.

In one embodiment, the cancer is selected from ovarian cancer, rectal cancer and renal cell carcinoma.

In some embodiments, the agent that detects gamma-H2AX expression levels is a reagent used in a ligand binding assay (LBA).

In some embodiments, the agent that detects gamma-H2AX expression levels is a reagent used in a protein immunostaining assay, e.g., immunohistochemistry assay, or e.g., an immunofluorescence assay.

In some embodiments, the agent that detects gamma-H2AX expression levels is a reagent used in a western blot assay, or in an ELISA assay.

In one embodiment, the agent detects gamma-H2AX protein, e.g., an anti-gamma-H2AX antibody.

In one embodiment, the agent detects a nucleic acid encoding gamma-H2AX, e.g., a probe that binds to a nucleic acid encoding gamma-H2AX.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: PAI-1, VEGF, CAIX, RAD51, topo 1 or topo 2. In one embodiment, the reaction mixture includes two or more additional agents (e.g., 2, 3, 4, 5 or 6 agents) selected from an agent that detects PAI-1, an agent that detects VEGF, an agent that detects CAM an agent that detects RAD51, an agent that detects topo 1 and an agent that detects topo 2.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects gamma-H2AX expression levels.

In one embodiment, the cancer is selected from ovarian cancer, rectal cancer and renal cell carcinoma.

In some embodiments, the agent that detects gamma-H2AX expression levels is a reagent used in a ligand binding assay (LBA).

In some embodiments, the agent that detects gamma-H2AX expression levels is a reagent used in a protein immunostaining assay, e.g., immunohistochemistry assay, or e.g., an immunofluorescence assay.

In some embodiments, the agent that detects gamma-H2AX expression levels is a reagent used in a western blot assay, or in an ELISA assay.

In one embodiment, the agent detects gamma-H2AX protein, e.g., an anti-gamma-H2AX antibody.

In one embodiment, the agent detects a nucleic acid encoding gamma-H2AX, e.g., a probe that binds to a nucleic acid encoding gamma-H2AX.

In one embodiment, the biological sample is a biopsy sample or a sample obtained from a lesion removed from the subject, e.g., a primary or metastatic lesion. In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

### RAD51

In yet another aspect, the disclosure features a method of treating a cancer in a subject, e.g., a human subject, the method comprising:

selecting a subject who has a cancer, e.g., a cancer associated with increased RAD51 expression levels, e.g., as compared to a reference standard (e.g., RAD51 levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating RAD51 expression levels in the sample as compared to a reference standard (e.g., RAD51 levels in a healthy subject or RAD51 levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the method further comprises determining the RAD51 expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of RAD51 expression can be determined by the amount of RAD51 nucleic acid (e.g., mRNA) in the sample, e.g., by any suitable method, e.g., northern blotting, RT-PCR, or the use of biochips.

In one embodiment, the level of RAD51 expression can be determined by the amount of RAD51 protein, e.g., by any suitable method.

In one embodiment, the level of RAD51 protein expression can be determined by a ligand binding assay (LBA).

In one embodiment, the level of RAD51 protein expression can be determined by protein immunostaining, e.g., immunohistochemistry, or e.g., immunofluorescence assay.

In one embodiment, the level of RAD51 protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased RAD51 expression levels, e.g., as compared to a reference standard (e.g., RAD51 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring RAD51 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the RAD51 expression levels in the subject are decreased as compared, e.g., to the RAD51 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial RAD51 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In yet another aspect, the disclosure features, a method of treating a cancer, in a subject, e.g., a human subject, the method comprising:

selecting a subject who has cancer, e.g., a cancer associated with increased RAD51 expression levels, e.g., as compared to a reference standard (e.g., RAD51 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring RAD51 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the RAD51 expression levels in the subject are the same or increased, e.g., as compared to the RAD51 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial RAD51 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased RAD51 expression levels, e.g., as compared to a reference standard (e.g., RAD51 levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating RAD51 expression levels in the biological sample, e.g., as compared to the reference standard or as compared to RAD51 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the RAD51 expression levels decrease as compared to the RAD51 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the RAD51 expression levels are the same or increased as compared to the RAD51 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of RAD51 expression can be determined by the amount of RAD51 nucleic acid (e.g., mRNA) in the sample, e.g., by any suitable method, e.g., northern blotting, RT-PCR, or the use of biochips.

In one embodiment, the level of RAD51 expression can be determined by the amount of RAD51 protein, e.g., by any suitable method.

In one embodiment, the level of RAD51 protein expression can be determined by a ligand binding assay (LBA).

In one embodiment, the level of RAD51 protein expression can be determined by protein immunostaining, e.g., immunohistochemistry, or e.g., immunofluorescence assay.

In one embodiment, the level of RAD51 protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects RAD51 expression levels.

In one embodiment, the cancer is selected from ovarian cancer, rectal cancer and renal cell carcinoma.

In some embodiments, the agent that detects RAD51 expression levels is a reagent used in a ligand binding assay (LBA).

In some embodiments, the agent that detects RAD51 expression levels is a reagent used in a protein immunostaining assay, e.g., immunohistochemistry assay, or e.g., an immunofluorescence assay.

In some embodiments, the agent that detects RAD51 expression levels is a reagent used in a western blot assay, or in an ELISA assay.

In one embodiment, the agent detects RAD51 protein, e.g., an anti-RAD51 antibody.

In one embodiment, the agent detects a nucleic acid encoding RAD51, e.g., a probe that binds to a nucleic acid encoding RAD51.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: PAI-1, VEGF, H2AX, CAIX, topo 1 or topo 2. In one embodiment, the reaction mixture includes two or more additional agents (e.g., 2, 3, 4, 5 or 6 agents) selected from an agent that detects PAI-1, an agent that detects VEGF, an agent that detects H2AX, an agent that detects CAM an agent that detects topo 1 and an agent that detects topo 2.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects RAD51 expression levels.

In one embodiment, the cancer is selected from ovarian cancer, rectal cancer and renal cell carcinoma.

In some embodiments, the agent that detects RAD51 expression levels is a reagent used in a ligand binding assay (LBA).

In some embodiments, the agent that detects RAD51 expression levels is a reagent used in a protein immunostaining assay, e.g., immunohistochemistry assay, or e.g., an immunofluorescence assay.

In some embodiments, the agent that detects RAD51 expression levels is a reagent used in a western blot assay, or in an ELISA assay.

In one embodiment, the agent detects RAD51 protein, e.g., an anti-RAD51 antibody.

In one embodiment, the agent detects a nucleic acid encoding RAD51, e.g., a probe that binds to a nucleic acid encoding RAD51.

In one embodiment, the biological sample is a biopsy sample or a sample obtained from a lesion removed from the subject, e.g., a primary or metastatic lesion. In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

### Topoisomerase-1 (Topo1)

In yet another aspect, the disclosure features a method of treating a cancer in a subject, e.g., a human subject, the method comprising:

selecting a subject who has a cancer, e.g., a cancer associated with increased Topo1 expression levels, e.g., as compared to a reference standard (e.g., Topo1 levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating Topo1 expression levels in the sample as compared to a reference standard (e.g., Topo1 levels in a healthy subject or Topo1 levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the method further comprises determining the Topo1 expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of Topo1 expression can be determined by the amount of Topo1 nucleic acid (e.g., mRNA) in the sample, e.g., by any suitable method, e.g., northern blotting, RT-PCR, or the use of biochips.

In one embodiment, the level of Topo1 expression can be determined by the amount of Topo1 protein, e.g., by any suitable method.

In one embodiment, the level of Topo1 protein expression can be determined by a ligand binding assay (LBA).

In one embodiment, the level of Topo1 protein expression can be determined by protein immunostaining, e.g., immunohistochemistry, or e.g., immunofluorescence assay.

In one embodiment, the level of Topo1 protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased Topo1 expression levels, e.g., as compared to a reference standard (e.g., Topo1 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring Topo1 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the Topo1 expression levels in the subject are decreased as compared, e.g., to the Topo1 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial Topo1 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In yet another aspect, the disclosure features, a method of treating a cancer, in a subject, e.g., a human subject, the method comprising:

selecting a subject who has cancer, e.g., a cancer associated with increased Topo1 expression levels, e.g., as compared to a reference standard (e.g., Topo1 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring Topo1 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the Topo1 expression levels in the subject are the same or increased, e.g., as compared to the Topo1 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial Topo1 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased Topo1 expression levels, e.g., as compared to a reference standard (e.g., Topo1 levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating Topo1 expression levels in the biological sample, e.g., as compared to the reference standard or as compared to Topo1 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the Topo1 expression levels decrease as compared to the Topo1 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the Topo1 expression levels are the same or increased as compared to the Topo1 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of Topo1 expression can be determined by the amount of Topo1 nucleic acid (e.g., mRNA) in the sample, e.g., by any suitable method, e.g., northern blotting, RT-PCR, or the use of biochips.

In one embodiment, the level of Topo1 expression can be determined by the amount of Topo1 protein, e.g., by any suitable method.

In one embodiment, the level of Topo1 protein expression can be determined by a ligand binding assay (LBA).

In one embodiment, the level of Topo1 protein expression can be determined by protein immunostaining, e.g., immunohistochemistry, or e.g., immunofluorescence assay.

In one embodiment, the level of Topo1 protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects Topo1 expression levels.

In one embodiment, the cancer is selected from ovarian cancer, rectal cancer and renal cell carcinoma.

In some embodiments, the agent that detects Topo1 expression levels is a reagent used in a ligand binding assay (LBA).

In some embodiments, the agent that detects Topo1 expression levels is a reagent used in a protein immunostaining assay, e.g., immunohistochemistry assay, or e.g., an immunofluorescence assay.

In some embodiments, the agent that detects Topo1 expression levels is a reagent used in a western blot assay, or in an ELISA assay.

In one embodiment, the agent detects Topo1 protein, e.g., an anti-Topo1 antibody.

In one embodiment, the agent detects a nucleic acid encoding Topo1, e.g., a probe that binds to a nucleic acid encoding Topo1.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: PAI-1, VEGF, H2AX, RAD51, CAIX or topo 2. In one embodiment, the reaction mixture includes two or more additional agents (e.g., 2, 3, 4, 5 or 6 agents) selected from an agent that detects PAI-1, an agent that detects VEGF, an agent that detects H2AX, an agent that detects RAD51, an agent that detects CAIX and an agent that detects topo 2.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects Topo1 expression levels.

In one embodiment, the cancer is selected from ovarian cancer, rectal cancer and renal cell carcinoma.

In some embodiments, the agent that detects Topo1 expression levels is a reagent used in a ligand binding assay (LBA).

In some embodiments, the agent that detects Topo1 expression levels is a reagent used in a protein immunostaining assay, e.g., immunohistochemistry assay, or e.g., an immunofluorescence assay.

In some embodiments, the agent that detects Topo1 expression levels is a reagent used in a western blot assay, or in an ELISA assay.

In one embodiment, the agent detects Topo1 protein, e.g., an anti-Topo1 antibody.

In one embodiment, the agent detects a nucleic acid encoding Topo1, e.g., a probe that binds to a nucleic acid encoding Topo1.

In one embodiment, the biological sample is a biopsy sample or a sample obtained from a lesion removed from the subject, e.g., a primary or metastatic lesion. In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

### Topoisomerase-2 (Topo2)

In yet another aspect, the disclosure features a method of treating a cancer in a subject, e.g., a human subject, the method comprising:

selecting a subject who has a cancer, e.g., a cancer associated with increased Topo2 expression levels, e.g., as compared to a reference standard (e.g., Topo2 levels of a healthy subject that does not have cancer); and administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the cancer is selected from ovarian cancer, rectal cancer and renal cell carcinoma.

In yet another aspect, the disclosure features, a method of treating cancer in a subject, e.g., a human subject, the method comprising:

a) optionally, acquiring, e.g., a biological sample, e.g., a tissue sample or blood sample, from a subject e.g., human subject, that has received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

b) acquiring an evaluation of and/or evaluating Topo2 expression levels in the sample as compared to a reference standard (e.g., Topo2 levels in a healthy subject or Topo2 levels in the subject prior to receiving an initial administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101); and

c) administering to the subject a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In one embodiment, the method further comprises determining the Topo2 expression levels after one or more of the administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the level of Topo2 expression can be determined by the amount of Topo2 nucleic acid (e.g., mRNA) in the sample, e.g., by any suitable method, e.g., northern blotting, RT-PCR, or the use of biochips.

In one embodiment, the level of Topo2 expression can be determined by the amount of Topo2 protein, e.g., by any suitable method.

In one embodiment, the level of Topo2 protein expression can be determined by a ligand binding assay (LBA).

In one embodiment, the level of Topo2 protein expression can be determined by protein immunostaining, e.g., immunohistochemistry, or e.g., immunofluorescence assay.

In one embodiment, the level of Topo2 protein expression can be determined by western blot or enzyme-linked immunosorbent assay (ELISA).

In yet another aspect, the disclosure features a method of treating a cancer, in a subject, e.g., a human subject, the method comprising:

selecting a subject, e.g., human subject, who has cancer, e.g., a cancer associated with increased Topo2 expression levels, e.g., as compared to a reference standard (e.g., Topo2 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring Topo2 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the Topo2 expression levels in the subject are decreased as compared, e.g., to the Topo2 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

providing one or more administrations of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial Topo2 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is provided at a dose and/or dosing schedule described herein.

In yet another aspect, the disclosure features, a method of treating a cancer, in a subject, e.g., a human subject, the method comprising:

selecting a subject who has cancer, e.g., a cancer associated with increased Topo2 expression levels, e.g., as compared to a reference standard (e.g., Topo2 levels of a healthy subject that does not have cancer) and has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101;

acquiring Topo2 expression levels in the subject after one or more administrations of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; wherein the Topo2 expression levels in the subject are the same or increased, e.g., as compared to the Topo2 expression levels in the subject prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101; and

administering a cancer treatment other than a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject, to thereby treat the cancer.

In one embodiment, the method further comprises acquiring an initial Topo2 expression level in the subject prior to treatment with the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the cancer is selected from ovarian cancer, rectal cancer and renal cell carcinoma.

In one embodiment, the cancer treatment, e.g., chemotherapeutic agent, other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is a chemotherapeutic agent or a combination of chemotherapeutic agents described herein.

In some embodiments, the cancer is ovarian cancer, and the chemotherapeutic agent is selected from imatinib, docetaxel, cabazitaxel, niraparib, paclitaxel, carboplatin, cisplatin, votinostat, veliparib, topotecan, AZ2281, lenalidomide, doxorubicin, bevacizumab, bendamustine, N-acetylcysteine, olaparib, rucaparib, AZD0530, lovastatin, flutamide, SU5416, CP-4055, MORAb-003 (farletuzumab), sagopilone (ZK 219477), sorafenib, panitumumab, trabectedin, KHK2866, gemcitabine, catumaxomab, melphalan, celecoxib, aflibercept, and defactinib (VS-6063).

In some embodiments, the cancer is rectal cancer, and the chemotherapeutic agent is selected from bevacizumab, tomudex, capecitabine, irinotecan, trametinib, florouracil, oxaliplatin, leucovorin, celecoxib, cetuximab, leucovorin, panitumumab, paricalcitol, ganetespib, udenafil, L-BLP25 (Stimuvax), midostaurin (PKC412), hydralazine, and rifaximin.

In some embodiments, the cancer is renal cell carcinoma, and the chemotherapeutic agent is selected from dovitinib, everolimus, aldesleukin, cyclophosphamide, fludarabine, zanolimumab, aldesleukin, naltrexone, lutetium-177, pazopanib, sorafenib, vandetanib, bevacizumab, erlotinib, sunitinib, velcade, alemtuzumab, gefitinib, vorinostat, isotretinoin, capecitabine, gemcitabine, doxorubicin, ipilimumab, MK2206, carfilzomib, and celecoxib.

In yet another aspect, the disclosure features, a method of evaluating the efficacy of a treatment, the method comprising:

acquiring, e.g., a biological sample, e.g., a tissue sample or blood sample, from a subject e.g., human subject, having cancer, e.g., a cancer associated with increased Topo2 expression levels, e.g., as compared to a reference standard (e.g., Topo2 levels of a healthy subject that does not have a cancer), and who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

evaluating Topo2 expression levels in the biological sample, e.g., as compared to the reference standard or as compared to Topo2 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, to thereby evaluate the efficacy of the treatment.

In one embodiment, the method further comprises the administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, at a dose or dosing schedule described herein.

In one embodiment, the Topo2 expression levels decrease as compared to the Topo2 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as effective.

In one embodiment, the Topo2 expression levels are the same or increased as compared to the Topo2 expression levels prior to administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, and the treatment is classified as ineffective, and/or a cancer treatment other than the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101, is selected for treatment.

In one embodiment, the level of Topo2 expression can be determined by the amount of Topo2 nucleic acid (e.g., mRNA) in the sample, e.g., by any suitable method, e.g., northern blotting, RT-PCR, or the use of biochips.

In one embodiment, the level of Topo2 expression can be determined by the amount of Topo2 protein, e.g., by any suitable method.

In one embodiment, the level of Topo2 protein expression can be determined by a ligand binding assay (LBA).

In one embodiment, the level of Topo2 protein expression can be determined by protein immunostaining, e.g., immunohistochemistry, or e.g., immunofluorescence assay.

In one embodiment, the level of Topo2 protein expression can be determined by western blot or enzyme-linked immunosorbent assay (ELISA).

In one embodiment, the cancer is selected from ovarian cancer, rectal cancer and renal cell carcinoma.

In yet another aspect, the disclosure features, a reaction mixture, comprising:

a biological sample, e.g., a tissue sample or blood sample, from a subject, e.g., a human subject, having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; and

an agent that detects Topo2 expression levels.

In one embodiment, the cancer is selected from ovarian cancer, rectal cancer and renal cell carcinoma.

In some embodiments, the agent that detects Topo2 expression levels is a reagent used in a ligand binding assay (LBA).

In some embodiments, the agent that detects Topo2 expression levels is a reagent used in a protein immunostaining assay, e.g., immunohistochemistry assay, or e.g., an immunofluorescence assay.

In some embodiments, the agent that detects Topo2 expression levels is a reagent used in a western blot assay, or in an ELISA assay.

In one embodiment, the agent detects Topo2 protein, e.g., an anti-Topo2 antibody.

In one embodiment, the agent detects a nucleic acid encoding Topo2, e.g., a probe that binds to a nucleic acid encoding Topo2.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the reaction mixture comprises an additional agent, e.g., an agent that detects: PAI-1, VEGF, H2AX, RAD51, topo 1 or CALX. In one embodiment, the reaction mixture includes two or more additional agents (e.g., 2, 3, 4, 5 or 6 agents) selected from an agent that detects PAI-1, an agent that detects VEGF, an agent that detects H2AX, an agent that detects RAD51, an agent that detects topo 1 and an agent that detects CAIX.

In yet another aspect, the disclosure features, a method of making a reaction mixture, comprising:

combining a biological sample, e.g., a tissue sample or blood sample, from a subject, e.g., a human subject having cancer, who has already received at least one administration of a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101; with an agent that detects Topo2 expression levels.

In one embodiment, the cancer is selected from ovarian cancer, rectal cancer and renal cell carcinoma.

In some embodiments, the agent that detects Topo2 expression levels is a reagent used in a ligand binding assay (LBA).

In some embodiments, the agent that detects Topo2 expression levels is a reagent used in a protein immunostaining assay, e.g., immunohistochemistry assay, or e.g., an immunofluorescence assay.

In some embodiments, the agent that detects Topo2 expression levels is a reagent used in a western blot assay, or in an ELISA assay.

In one embodiment, the agent detects Topo2 protein, e.g., an anti-Topo2 antibody.

In one embodiment, the agent detects a nucleic acid encoding Topo2, e.g., a probe that binds to a nucleic acid encoding Topo2.

In one embodiment, the biological sample is a biopsy sample or a sample obtained from a lesion removed from the subject, e.g., a primary or metastatic lesion.

In some embodiments, the sample is obtained concurrently with the CDP-topoisomerase inhibitor conjugate, particle or composition, administration treatment and/or post-treatment, e.g., 1, 2, 3, 4, 5, 6, 7, 8, 9, or 10 weeks after the last administration of the CDP-topoisomerase inhibitor conjugate, particle or composition, e.g., CDP-camptothecin or camptothecin derivative conjugate, particle or composition, e.g., CDP-camptothecin or camptothecin derivative conjugate, particle or composition described herein, e.g., CRLX101.

In one embodiment, the CDP-topoisomerase inhibitor conjugate, particle or composition, e.g., a CDP-camptothecin conjugate, particle or composition or camptothecin derivative conjugate, particle or composition, e.g., a CDP-camptothecin conjugate, particle or composition or camptothecin derivative conjugate, particle or composition described herein, e.g., CRLX101, decreases HIF-1α levels, HIF-2α levels, or both in the subject having cancer. In some embodiments, HIF-1α levels, HIF-2α levels, or both are compared to a reference standard, e.g., HIF-1α levels, HIF-2α levels, or both in a healthy subject that does not have cancer. In one embodiment, the method includes selecting a subject having increased HIF-1α levels, HIF-2α levels, or both (e.g., as compared to a reference standard) for treatment with the conjugate, particle or composition. In one embodiment, the method includes selecting a subject having or at risk of becoming resistant to treatment with a chemotherapeutic agent, e.g., the subject is at risk of developing hypoxia-induced resistance to a chemotherapeutic agent, for treatment with the, particle or composition. In one embodiment, the method includes selecting a subject having or at risk of developing a metastases. In one embodiment, the method comprises administering the conjugate, particle or composition in combination with an agent that increases HIF-1α levels, HIF-2α levels, or both.

In one embodiment, the method further comprises administering an antiemetic, e.g., a 5-HT3 receptor antagonist (e.g., zofran).

In one embodiment, the method further comprises administering an agent which ameliorates bladder toxicity associated with therapy, e.g., an agent which increases urinary excretion and/or neutralizes one or more urinary metabolite. In one embodiment, the agent which ameliorates bladder toxicity associated with therapy is saline, e.g., intravenous saline, D5 half normal saline or D5 water. In one embodiment, the agent which increases urinary excretion and/or neutralizes one or more urinary metabolite is 2-mercaptoethane sulfonate sodium (MESNA).

In one embodiment, the method further comprises administering a growth factor, e.g., a hematopoietic growth factor such as, e.g., granulocyte colony stimulating factor (GCSF) or granulocyte macrophage colony stimulating factor (GM-CSF).

In one embodiment, the method further comprises administration of an antihypertension agent.

In one aspect, the disclosure features, a method of treating a proliferative disorder, e.g., a cancer, in a subject, e.g., a human subject. The method comprises:

providing a subject who has a proliferative disorder, e.g., cancer, associated with an increased level of HIF-1α and/or HIF-2α; and

administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject.

In an embodiment, the conjugate includes a topoisomerase I inhibitor and/or a topoisomerase II inhibitor. In an embodiment, the conjugate includes a topoisomerase I inhibitor or combination of topoisomerase I inhibitors, e.g., camptothecin, irinotecan, SN-38, topotecan, lamellarin D and derivatives thereof. In an embodiment, the conjugate includes a topoisomerase II inhibitor or a combination of topoisomerase II inhibitors, e.g., etoposide, tenoposide, doxorubicin and derivatives thereof. In one embodiment, the conjugate includes a combination of one or more topoisomerase I inhibitors and one or more topoisomerase II inhibitors. In an embodiment, the CDP-topoisomerase inhibitor conjugate is a CDP-camptothecin or camptothecin derivate conjugate, e.g., a CDP-camptothecin or camptothecin derivative conjugate described herein, e.g., CRLX101.

In one embodiment, the proliferative disorder is cancer, e.g., a cancer described herein.

In one embodiment, the CDP-topoisomerase inhibitor conjugate, particle or composition, e.g., a CDP-topoisomerase inhibitor conjugate, particle or composition, described herein, e.g., a CDP-camptothecin or camptothecin derivative conjugate, particle or composition, e.g., a CDP-camptothecin or camptothecin derivative conjugate, particle or composition described herein, is administered in combination with one or more additional chemotherapeutic agent, e.g., as described herein. In one embodiment, the method comprises administering the conjugate, particle or composition in combination with an agent that increases HIF-1α and/or HIF-2α levels. In one embodiment, the CDP-topoisomerase inhibitor conjugate, particle or composition is administered at a dose and/or dosing schedule described herein.

In one embodiment, the CDP-topoisomerase inhibitor conjugate, particle or composition is further administered in combination with one or more of the agents described herein. For example, the CDP-topoisomerase inhibitor conjugate, particle or composition can be administered in combination with an agent which reduces or inhibits one or more symptom of hypersensitivity.

In yet another aspect, the disclosure features a method of treating a subject, e.g., a human subject, with a proliferative disorder, e.g., cancer, comprising:

selecting a subject who has a proliferative disorder, e.g., cancer, that has increased HIF-2α levels, e.g., as compared to a reference standard (e.g., HIF-2α levels of a healthy subject that does not have cancer); and

administering a CDP-topoisomerase inhibitor conjugate, particle or composition, e.g., a CDP-camptothecin or camptothecin derivative conjugate, particle or composition, e.g., a CDP-camptothecin or camptothecin derivative conjugate, particle or composition described herein, e.g., CRLX101, to the subject in an amount effective to treat the cancer, to thereby treat the cancer.

In an embodiment, the conjugate includes a topoisomerase I inhibitor and/or a topoisomerase II inhibitor. In an embodiment, the conjugate includes a topoisomerase I inhibitor or combination of topoisomerase I inhibitors, e.g., camptothecin, irinotecan, SN-38, topotecan, lamellarin D and derivatives thereof. In an embodiment, the conjugate includes a topoisomerase II inhibitor or a combination of topoisomerase II inhibitors, e.g., etoposide, tenoposide, doxorubicin and derivatives thereof. In one embodiment, the conjugate includes a combination of one or more topoisomerase I inhibitors and one or more topoisomerase II inhibitors. In an embodiment, the CDP-topoisomerase inhibitor conjugate is a CDP-camptothecin or camptothecin derivate conjugate, e.g., a CDP-camptothecin or camptothecin derivative conjugate described herein, e.g., CRLX101.

In one embodiment, the subject has lung cancer (e.g., small cell lung cancer and/or non-small cell lung cancer) or kidney cancer (e.g., renal cell carcinoma).

In one embodiment, the cancer is ovarian cancer.

In one embodiment, the cancer is a cancer described herein.

In one embodiment, the CDP-topoisomerase inhibitor conjugate, particle or composition is administered in combination with one or more additional chemotherapeutic agent, e.g., as described herein. In one embodiment, the CDP-topoisomerase inhibitor conjugate, particle or composition is administered at a dose and/or dosing schedule described herein.

In one embodiment, the CDP-topoisomerase inhibitor conjugate, particle or composition is administered in combination with one or more of the agents described herein. For example, the CDP-topoisomerase inhibitor conjugate, particle or composition can be administered in combination with an agent which reduces or inhibits one or more symptom of hypersensitivity and/or an agent which increases urinary excretion and/or neutralizes one or more urinary metabolite.

The details of one or more embodiments of the invention are set forth in the description below. Other features, objects, and advantages of the invention will be apparent from the description and the drawings, and from the claims.

### BRIEF DESCRIPTION OF THE DRAWINGS

FIGS. 1A and 1B are CT (computed tomography) scans from a patient with metastatic pancreatic cancer pre-treatment (FIG. 1A), and after six months of treatment with CRLX101 (FIG. 1B). The patient received 6 mg/m² CRLX101 on schedule Ia.

FIGS. 2A and 2B are graphs depicting pharmacokinetic and toxicokinetic analysis of CRLX101 delivered by intravenous administration. FIG. 2A shows mean plasma concentration-time profile for polymer conjugated (squares) and unconjugated (triangles) CPT for cohort 1b-1 (12 mg/m²). FIG. 2B shows average urinary excretion of polymer conjugated (black bars) and unconjugated CPT (white bars) in the first 48 hours following CRLX101 administration. Plasma concentrations for conjugated and unconjugated CPT were below the limit of quantitation at 336 hrs (before the second dose) and therefore are not plotted in FIG. 2A.

FIGS. 3A-3C depict immunohistochemistry and topoisomerase I activity of ovarian cancer cells from a patient treated with CRLX101, showing post-treatment reduction of topoisomerase I protein. FIG. 3A is an immunohistochemical stain of ascites cells collected before CRLX101 was given. FIG. 3B is an immunohistochemical stain of ascites cells collected 2 days following CRLX101 treatment. FIG. 3C is a gel depicting the results of topoisomerase I enzymatic activity assay in whole cell lysates.

FIG. 4 depicts the structure and description of an exemplary CDP-camptothecin conjugate referred to as “CRLX101” throughout this application. CRLX101 is used interchangeably with the term CRLX101 (e.g., as in Example 1).

FIGS. 5A, 5B, and 5C depict the effects of CRLX101 on tumor growth in a non-small cell lung cancer mouse model (A549) possessing a KRAS mutation. FIG. 5A shows the effect of CRLX101 administered at 2 mg/kg qwk×3 (▾), 4 mg/kg qwk×3 (▪), and 6 mg/kg qwk×3 (▴) on tumor volume. FIG. 5B shows the effect of CRLX101 administered at 2 mg/kg qwk×3 (▾), 4 mg/kg qwk×3 (▪), and 6 mg/kg qwk×3 (▴) on body weight. FIG. 5C shows the effect of CRLX101 administered at 2 mg/kg qwk×3 (▾), 4 mg/kg qwk×3 (▪), and 6 mg/kg qwk×3 (▴) on survival.

FIGS. 6A, 6B, and 6C depict the effects of CRLX101 on tumor growth in a non-small cell lung cancer mouse model (NCI-H2122) possessing a KRAS mutation. FIG. 6A shows the effect of CRLX101 administered at 2 mg/kg q7d×3 (▾), 4 mg/kg q7d×3 (▪), and 6 mg/kg q7d×3 (▴) on tumor volume. FIG. 6B shows the effect of CRLX101 administered at 2 mg/kg q7d×3 (▾), 4 mg/kg q7d×3 (▪), and 6 mg/kg q7d×3 (▴) on body weight. FIG. 6C shows the effect of CRLX101 administered at 2 mg/kg q7d×3 (▾), 4 mg/kg q7d×3 (▪), and 6 mg/kg q7d×3 (▴) on survival.

FIGS. 7A, 7B and 7C compare the effect of administering a combination of CRLX101 and sorafenib on tumor growth in a non-small cell lung cancer mouse model (H1299). FIG. 7A shows the effect of CRLX101 administered alone at 6 mg/kg qwk×3, sorafenib administered at 60 mg/kg qd×21 and CRLX101 (6 mg/kg qwk×3) and sorafenib (60 mg/kg qd×21) on tumor volume. FIG. 7B shows the effect of CRLX101 administered at 6 mg/kg qwk×3, sorafenib administered at 60 mg/kg qd×21, and CRLX101 (6 mg/kg qwk×3) and sorafenib (60 mg/kg qd×21) on body weight. FIG. 7C shows the effect of CRLX101 administered at 6 mg/kg qwk×3, sorafenib administered at 60 mg/kg qd×21, and CRLX101 (6 mg/kg qwk×3) and sorafenib (60 mg/kg qd×21) on survival.

FIGS. 8A and 8B depict bar graphs showing CRLX101 inhibition of HIF-1α and HIF-2α, respectively, as compared to maximum tolerated doses (MTD) (10 mg/kg q4d×3) of topotecan and metronomic topotecan (1 mg/kg qd×8).

FIG. 9 is a line graph showing the tumor growth curves for vehicle (), CRLX101 (5 mg/kg) (▪), Bevacizumab (5 mg/kg) (▴), and CRLX101 (5 mg/kg)+Bevacizumab (5 mg/kg) (♦).

### DETAILED DESCRIPTION OF THE INVENTION

The present invention relates to compositions of therapeutic cyclodextrin-containing polymers (CDP) designed for drug delivery of a topoisomerase inhibitor such as camptothecin or a camptothecin derivative. In certain embodiments, these cyclodextrin-containing polymers improve drug stability and/or solubility, and/or reduce toxicity, and/or improve efficacy of the topoisomerase inhibitor when used in vivo.

Furthermore, by selecting from a variety of linker groups that link or couple CDP to a topoisomerase inhibitor such as camptothecin or a camptothecin derivative, and/or targeting ligands, the rate of drug release from the polymers can be attenuated for controlled delivery. The invention also relates to methods of treating subjects with compositions described herein. The invention further relates to methods for conducting a pharmaceutical business comprising manufacturing, licensing, or distributing kits containing or relating to the CDP-topoisomerase inhibitor conjugates, particles and compositions described herein.

More generally, the present invention provides water-soluble, biocompatible polymer conjugates comprising a water-soluble, biocompatible polymer covalently attached to the topoisomerase inhibitor through attachments that are cleaved under biological conditions to release the topoisomerase inhibitor.

Polymeric conjugates featured in the methods described herein may be useful to improve solubility and/or stability of a bioactive/therapeutic agent, such as camptothecin, reduce drug-drug interactions, reduce interactions with blood elements including plasma proteins, reduce or eliminate immunogenicity, protect the agent from metabolism, modulate drug-release kinetics, improve circulation time, improve drug half-life (e.g., in the serum, or in selected tissues, such as tumors), attenuate toxicity, improve efficacy, normalize drug metabolism across subjects of different species, ethnicities, and/or races, and/or provide for targeted delivery into specific cells or tissues.

In preferred embodiments, the topoisomerase inhibitor in the CDP-topoisomerase inhibitor conjugate, particle or composition is camptothecin or a camptothecin derivative. The term “camptothecin derivative”, as used herein, includes camptothecin analogues and metabolites of camptothecin. For example, camptothecin derivatives can have the following structure:

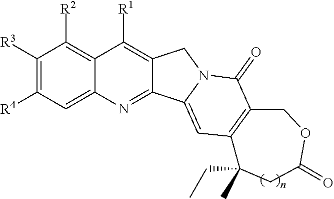

wherein

R¹ is H, OH, optionally substituted alkyl (e.g., optionally substituted with NR^{a}₂ or ORₐ, or SiR^{a}₃), or SiR^{a}₃; or R¹ and R² may be taken together to form an optionally substituted 5- to 8-membered ring (e.g., optionally substituted with NR^{a}₂ or OR^{a});

R² is H, OH, NH₂, halo, nitro, optionally substituted alkyl (e.g., optionally substituted with NR^{a}₂ or OR^{a}, NR^{a}₂, OC(═O)NR^{a}₂, or OC(═O)OR^{a});

R³ is H, OH, NH₂, halo, nitro, NR^{a}₂, OC(═O)NR^{a}₂, or OC(═O)OR^{a}

R⁴ is H, OH, NH₂, halo, CN, or NR^{a}₂; or R³ and R⁴ taken together with the atoms to which they are attached form a 5- or 6-membered ring (e.g. forming a ring including —OCH₂O— or —OCH₂CH₂O—);

each R^{a} is independently H or alkyl; or two R^{a}s, taken together with the atom to which they are attached, form a 4- to 8-membered ring (e.g., optionally containing an O or NR^{b})

R^{b} is H or optionally substituted alkyl (e.g., optionally substituted with OR^{c} or NR^{c}₂);

R^{c} is H or alkyl; or, two R^{c}s, taken together with the atom to which they are attached, form a 4- to 8-membered ring; and

n=0 or 1.

In some embodiments, the camptothecin or camptothecin derivative is the compound as provided below.

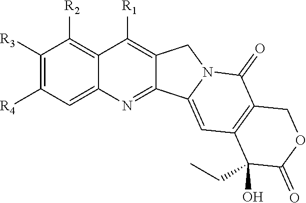

In one embodiment, R¹, R², R³ and R⁴ of the camptothecin derivative are each H, and n is 0.

In one embodiment, R¹, R², R³ and R⁴ of the camptothecin derivative are each H, and n is 1.

In one embodiment, R¹ of the camptothecin derivative is H, R² is —CH₂N(CH₃)₂, R³ is —OH, R⁴ is H; and n is 0.

In one embodiment, R¹ of the camptothecin derivative is —CH₂CH₃, R² is H, R³ is:

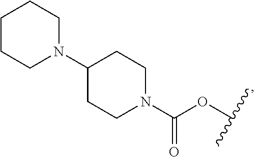 [SMILES: *OC(=O)N1CCC(N2CCCCC2)CC1]

### R⁴ is H, and n is 0.

In one embodiment, R¹ of the camptothecin derivative is —CH₂CH₃, R² is H,

R³ is —OH, R⁴ is H, and n is 0.

In one embodiment, R¹ of the camptothecin derivative is tert-butyldimethylsilyl, R² is H, R³ is —OH and R⁴ is H, and n is 0.

In one embodiment, R¹ of the camptothecin derivative is tert-butyldimethylsilyl, R² is hydrogen, R³ is —OH and R⁴ is hydrogen, and n is 1.

In one embodiment, R¹ of the camptothecin derivative is tert-butyldimethylsilyl, R², R³ and R⁴ are each H, and n is 0.

In one embodiment, R¹ of the camptothecin derivative is tert-butyldimethylsilyl, R², R³ and R⁴ are each H, and n is 1.

In one embodiment, R¹ of the camptothecin derivative is —CH₂CH₂Si(CH₃)₃ and R², R³ and R⁴ are each H.

In one embodiment, R¹ and R² of the camptothecin derivative are taken together with the carbons to which they are attached to form an optionally substituted ring. In one embodiment, R¹ and R² of the camptothecin derivative are taken together with the carbons to which they are attached to form a substituted 6-membered ring. In one embodiment, the camptothecin derivative has the following formula:

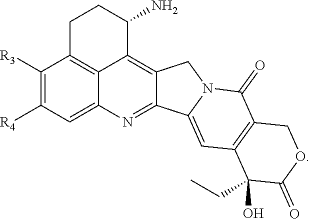

In one embodiment, R³ is methyl and R⁴ is fluoro.

In one embodiment, R³ and R⁴ are taken together with the carbons to which they are attached to form an optionally substituted ring. In one embodiment, R³ and R⁴ are taken together with the carbons to which they are attached to form a 6-membered heterocyclic ring. In one embodiment, the camptothecin derivative has the following formula:

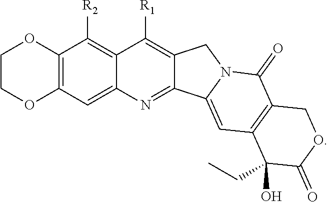

In one embodiment, R¹ is:

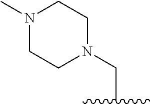 [SMILES: *CN1CCN(C)CC1]

and R² is hydrogen.

In one embodiment, the camptothecin derivative has the following formula:

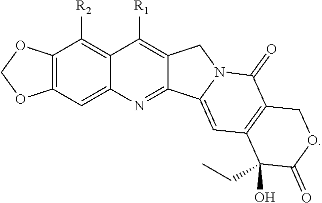

In one embodiment, R¹ is:

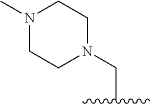 [SMILES: *CN1CCN(C)CC1]

and R² is hydrogen.

In one embodiment, R¹ is:

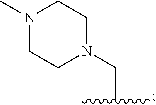 [SMILES: *CN1CCN(C)CC1]

R² is H, R³ is methyl, R⁴ is chloro; and n is 1.

In one embodiment, R¹ is —CH═NOC(CH₃)₃, R², R³ and R⁴ are each H, and n is 0.

In one embodiment, R¹ is —CH₂CH₂NHCH(CH₃)₂, R², R³ and R⁴ are each H; and n is 0.

In one embodiment, R¹ and R² are H, R³ and R⁴ are fluoro, and n is 1.

In one embodiment, each of R¹, R³, and R⁴ is H, R² is NH₂, and n is 0.

In one embodiment, each of R¹, R³, and R⁴ is H, R² is NO₂, and n is 0.

An “effective amount” or “an amount effective” refers to an amount of the CDP-topoisomerase inhibitor conjugate, particle or composition which is effective, upon single or multiple dose administrations to a subject, in treating a cell, or curing, alleviating, relieving or improving a symptom of a disorder. An effective amount of the conjugate, particle or composition may vary according to factors such as the disease state, age, sex, and weight of the individual, and the ability of the compound to elicit a desired response in the individual. An effective amount is also one in which any toxic or detrimental effects of the conjugate, particle or composition is outweighed by the therapeutically beneficial effects.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

As used herein, the term “treat” or “treating” a subject having a disorder refers to subjecting the subject to a regimen, e.g., the administration of a CDP-topoisomerase inhibitor conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

An amount of a CDP-topoisomerase inhibitor conjugate, particle or composition effective to prevent a disorder, or “a prophylactically effective amount” of the conjugate, particle or composition as used in the context of the administration of an agent to a subject, refers to subjecting the subject to a regimen, e.g., the administration of a CDP-topoisomerase inhibitor conjugate, particle or composition such that the onset of at least one symptom of the disorder is delayed as compared to what would be seen in the absence of the regimen.

### CDP-Topoisomerase Inhibitor Conjugates, Particles and Compositions

Described herein are cyclodextrin containing polymer (“CDP”)-topoisomerase inhibitor conjugates, wherein one or more topoisomerase inhibitors are covalently attached to the CDP (e.g., either directly or through a linker). The CDP-topoisomerase inhibitor conjugates include linear or branched cyclodextrin-containing polymers and polymers grafted with cyclodextrin. Exemplary cyclodextrin-containing polymers that may be modified as described herein are taught in U.S. Pat. Nos. 7,270,808, 6,509,323, 7,091,192, 6,884,789, U.S. Publication Nos. 20040087024, 20040109888 and 20070025952.

Accordingly, in one embodiment the CDP-topoisomerase inhibitor conjugate is represented by Formula I:

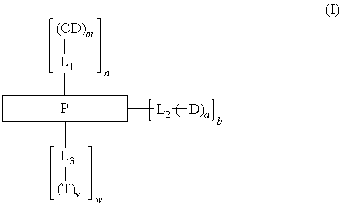

wherein

P represents a linear or branched polymer chain;

CD represents a cyclic moiety such as a cyclodextrin moiety;

L₁, L₂ and L₃, independently for each occurrence, may be absent or represent a linker group;

D, independently for each occurrence, represents a topoisomerase inhibitor or a prodrug thereof (e.g., a camptothecin or camptothecin derivative);

T, independently for each occurrence, represents a targeting ligand or precursor thereof;

a, m, and v, independently for each occurrence, represent integers in the range of 1 to 10 (preferably 1 to 8, 1 to 5, or even 1 to 3);

n and w, independently for each occurrence, represent an integer in the range of 0 to about 30,000 (preferably <25,000, <20,000, <15,000, <10,000, <5,000, <1,000, <500, <100, <50, <25, <10, or even <5); and

b represents an integer in the range of 1 to about 30,000 (preferably <25,000, <20,000, <15,000, <10,000, <5,000, <1,000, <500, <100, <50, <25, <10, or even <5),

wherein either P comprises cyclodextrin moieties or n is at least 1.

In some embodiments, one or more of the topoisomerase inhibitor moieties in the CDP-topoisomerase inhibitor conjugate can be replaced with another therapeutic agent, e.g., another anticancer agent or anti-inflammatory agent. Examples of other anticancer agents are described herein. Examples of anti-inflammatory agents include a steroid, e.g., prednisone, and a NSAID.

In certain embodiments, P contains a plurality of cyclodextrin moieties within the polymer chain as opposed to the cyclodextrin moieties being grafted on to pendant groups off of the polymeric chain. Thus, in certain embodiments, the polymer chain of formula I further comprises n′ units of U, wherein n′ represents an integer in the range of 1 to about 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5); and U is represented by one of the general formulae below:

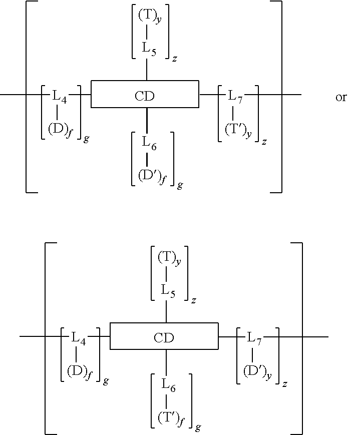

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different topoisomerase inhibitor or prodrug forms thereof (e.g., a camptothecin or camptothecin derivative);

T and T′, independently for each occurrence, represent the same or different targeting ligand or precursor thereof;

f and y, independently for each occurrence, represent an integer in the range of 1 and 10; and

g and z, independently for each occurrence, represent an integer in the range of 0 and 10.

Preferably the polymer has a plurality of D or D′ moieties. In some embodiments, at least 50% of the U units have at least one D or D′. In some embodiments, one or more of the topoisomerase inhibitor moieties in the CDP-topoisomerase conjugate can be replaced with another therapeutic agent, e.g., another anticancer agent or anti-inflammatory agent.

In preferred embodiments, L₄ and L₇ represent linker groups.

The CDP may include a polycation, polyanion, or non-ionic polymer. A polycationic or polyanionic polymer has at least one site that bears a positive or negative charge, respectively. In certain such embodiments, at least one of the linker moiety and the cyclic moiety comprises such a charged site, so that every occurrence of that moiety includes a charged site. In some embodiments, the CDP is biocompatible.

In certain embodiments, the CDP may include polysaccharides, and other non-protein biocompatible polymers, and combinations thereof, that contain at least one terminal hydroxyl group, such as polyvinylpyrrollidone, poly(oxyethylene)glycol (PEG), polysuccinic anhydride, polysebacic acid, PEG-phosphate, polyglutamate, polyethylenimine, maleic anhydride divinylether (DIVMA), cellulose, pullulans, inulin, polyvinyl alcohol (PVA), N-(2-hydroxypropyl)methacrylamide (HPMA), dextran and hydroxyethyl starch (HES), and have optional pendant groups for grafting therapeutic agents, targeting ligands and/or cyclodextrin moieties. In certain embodiments, the polymer may be biodegradable such as poly(lactic acid), poly(glycolic acid), poly(alkyl 2-cyanoacrylates), polyanhydrides, and polyorthoesters, or bioerodible such as polylactide-glycolide copolymers, and derivatives thereof, non-peptide polyaminoacids, polyiminocarbonates, poly alpha-amino acids, polyalkyl-cyano-acrylate, polyphosphazenes or acyloxymethyl poly aspartate and polyglutamate copolymers and mixtures thereof.

In another embodiment the CDP-topoisomerase inhibitor conjugate is represented by Formula II:

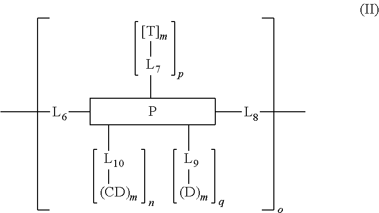

wherein

P represents a monomer unit of a polymer that comprises cyclodextrin moieties;

T, independently for each occurrence, represents a targeting ligand or a precursor thereof;

L₆, L₇, L₈, L₉, and L₁₀, independently for each occurrence, may be absent or represent a linker group;

CD, independently for each occurrence, represents a cyclodextrin moiety or a derivative thereof;

D, independently for each occurrence, represents a topoisomerase inhibitor or a prodrug form thereof (e.g., a camptothecin or camptothecin derivative);

m, independently for each occurrence, represents an integer in the range of 1 to 10 (preferably 1 to 8, 1 to 5, or even 1 to 3);

o represents an integer in the range of 1 to about 30,000 (preferably <25,000, <20,000, <15,000, <10,000, <5,000, <1,000, <500, <100, <50, <25, <10, or even <5); and

p, n, and q, independently for each occurrence, represent an integer in the range of 0 to 10 (preferably 0 to 8, 0 to 5, 0 to 3, or even 0 to about 2),

wherein CD and D are preferably each present at least 1 location (preferably at least 5, 10, 25, or even 50 or 100 locations) in the compound.

In some embodiments, one or more of the topoisomerase inhibitor moieties in the CDP-topoisomerase inhibitor conjugate can be replaced with another therapeutic agent, e.g., another anticancer agent or anti-inflammatory agent. Examples of an anticancer agent are described herein. Examples of anti-inflammatory agents include a steroid, e.g., prednisone, or a NSAID.

In another embodiment the CDP-topoisomerase inhibitor conjugate is represented either of the formulae below:

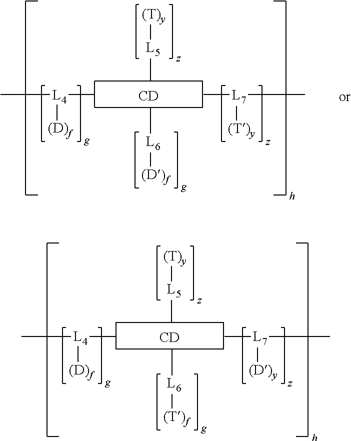

wherein

CD represents a cyclic moiety, such as a cyclodextrin moiety, or derivative thereof;

L₄, L₅, L₆, and L₇, independently for each occurrence, may be absent or represent a linker group;

D and D′, independently for each occurrence, represent the same or different topoisomerase inhibitor or prodrug thereof (e.g., a camptothecin or camptothecin derivative);

T and T′, independently for each occurrence, represent the same or different targeting ligand or precursor thereof;

f and y, independently for each occurrence, represent an integer in the range of 1 and 10 (preferably 1 to 8, 1 to 5, or even 1 to 3);

g and z, independently for each occurrence, represent an integer in the range of 0 and 10 (preferably 0 to 8, 0 to 5, 0 to 3, or even 0 to about 2); and

h represents an integer in the range of 1 and 30,000, e.g., from 4-100, 4-50, 4-25, 4-15, 6-100, 6-50, 6-25, and 6-15 (preferably <25,000, <20,000, <15,000, <10,000, <5,000, <1,000, <500, <100, <50, <25, <20, <15, <10, or even <5),

wherein at least one occurrence (and preferably at least 5, 10, or even at least 20, 50, or 100 occurrences) of g represents an integer greater than 0.

Preferably the polymer has a plurality of D or D′ moieties. In some embodiments, at least 50% of the polymer repeating units have at least one D or D′. In some embodiments, one or more of the topoisomerase inhibitor moieties in the CDP-topoisomerase inhibitor conjugate can be replaced with another therapeutic agent, e.g., another anticancer agent or anti-inflammatory agent.

In preferred embodiments, L₄ and L₇ represent linker groups.

In certain such embodiments, the CDP comprises cyclic moieties alternating with linker moieties that connect the cyclic structures, e.g., into linear or branched polymers, preferably linear polymers. The cyclic moieties may be any suitable cyclic structures, such as cyclodextrins, crown ethers (e.g., 18-crown-6,15-crown-5,12-crown-4, etc.), cyclic oligopeptides (e.g., comprising from 5 to 10 amino acid residues), cryptands or cryptates (e.g., cryptand [2.2.2], cryptand-2,1,1, and complexes thereof), calixarenes, or cavitands, or any combination thereof. Preferably, the cyclic structure is (or is modified to be) water-soluble. In certain embodiments, e.g., for the preparation of a linear polymer, the cyclic structure is selected such that under polymerization conditions, exactly two moieties of each cyclic structure are reactive with the linker moieties, such that the resulting polymer comprises (or consists essentially of) an alternating series of cyclic moieties and linker moieties, such as at least four of each type of moiety. Suitable difunctionalized cyclic moieties include many that are commercially available and/or amenable to preparation using published protocols. In certain embodiments, conjugates are soluble in water to a concentration of at least 0.1 g/mL, preferably at least 0.25 g/mL.

Thus, in certain embodiments, the invention relates to novel compositions of therapeutic cyclodextrin-containing polymeric compounds designed for drug delivery of a topoisomerase inhibitor. In certain embodiments, these CDPs improve drug stability and/or solubility, and/or reduce toxicity, and/or improve efficacy of the topoisomerase inhibitor when used in vivo. Furthermore, by selecting from a variety of linker groups, and/or targeting ligands, the rate of topoisomerase inhibitor release from the CDP can be attenuated for controlled delivery.

In certain embodiments, the CDP comprises a linear cyclodextrin-containing polymer, e.g., the polymer backbone includes cyclodextrin moieties. For example, the polymer may be a water-soluble, linear cyclodextrin polymer produced by providing at least one cyclodextrin derivative modified to bear one reactive site at each of exactly two positions, and reacting the cyclodextrin derivative with a linker having exactly two reactive moieties capable of forming a covalent bond with the reactive sites under polymerization conditions that promote reaction of the reactive sites with the reactive moieties to form covalent bonds between the linker and the cyclodextrin derivative, whereby a linear polymer comprising alternating units of cyclodextrin derivatives and linkers is produced. Alternatively the polymer may be a water-soluble, linear cyclodextrin polymer having a linear polymer backbone, which polymer comprises a plurality of substituted or unsubstituted cyclodextrin moieties and linker moieties in the linear polymer backbone, wherein each of the cyclodextrin moieties, other than a cyclodextrin moiety at the terminus of a polymer chain, is attached to two of said linker moieties, each linker moiety covalently linking two cyclodextrin moieties. In yet another embodiment, the polymer is a water-soluble, linear cyclodextrin polymer comprising a plurality of cyclodextrin moieties covalently linked together by a plurality of linker moieties, wherein each cyclodextrin moiety, other than a cyclodextrin moiety at the terminus of a polymer chain, is attached to two linker moieties to form a linear cyclodextrin polymer.

In some embodiments, the CDP-topoisomerase inhibitor conjugate comprises a water soluble linear polymer conjugate comprising: cyclodextrin moieties; comonomers which do not contain cyclodextrin moieties (comonomers); and a plurality of topoisomerase inhibitor; wherein the CDP-topoisomerase inhibitor conjugate comprises at least four, five six, seven, eight, etc., cyclodextrin moieties and at least four, five six, seven, eight, etc., comonomers. In some embodiments, the topoisomerase inhibitor is a topoisomerase inhibitor described herein, for example, the topoisomerase inhibitor is a camptothecin or camptothecin derivative described herein. The topoisomerase inhibitor can be attached to the CDP via a functional group such as a hydroxyl group, or where appropriate, an amino group.

In some embodiments, one or more of the topoisomerase inhibitor moieties in the CDP-topoisomerase inhibitor conjugate can be replaced with another therapeutic agent, e.g., another anticancer agent or anti-inflammatory agent.

In some embodiments, the least four cyclodextrin moieties and at least four comonomers alternate in the CDP-topoisomerase inhibitor conjugate. In some embodiments, the topoisomerase inhibitors are cleaved from the CDP-topoisomerase inhibitor conjugate under biological conditions to release the topoisomerase inhibitor. In some embodiments, the cyclodextrin moieties comprise linkers to which topoisomerase inhibitors are linked. In some embodiments, the topoisomerase inhibitors are attached via linkers.

In some embodiments, the comonomer comprises residues of at least two functional groups through which reaction and linkage of the cyclodextrin monomers was achieved. In some embodiments, the functional groups, which may be the same or different, terminal or internal, of each comonomer comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a topoisomerase inhibitor was achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring. In some embodiments, the cyclodextrin moiety comprises an alpha, beta, or gamma cyclodextrin moiety. In some embodiments, the topoisomerase inhibitor is at least 5%, 10%, 15%, 20%, 25%, 30%, or 35% by weight of CDP-topoisomerase inhibitor conjugate.

In some embodiments, the comonomer comprises polyethylene glycol of molecular weight 3,400 Da, the cyclodextrin moiety comprises beta-cyclodextrin, the theoretical maximum loading of the topoisomerase inhibitor on the CDP-topoisomerase inhibitor conjugate is 13% by weight, and the topoisomerase inhibitor is 6-10% by weight of CDP-topoisomerase inhibitor conjugate. In some embodiments, the topoisomerase inhibitor is poorly soluble in water. In some embodiments, the solubility of the topoisomerase inhibitor is <5 mg/ml at physiological pH. In some embodiments, the topoisomerase inhibitor is a hydrophobic compound with a log P>0.4, >0.6, >0.8, >1, >2, >3, >4, or >5.

In some embodiments, the topoisomerase inhibitor is attached to the CDP via a second compound.

In some embodiments, administration of the CDP-topoisomerase inhibitor conjugate to a subject results in release of the topoisomerase inhibitor over a period of at least 6 hours. In some embodiments, administration of the CDP-topoisomerase inhibitor conjugate to a subject results in release of the topoisomerase inhibitor over a period of 2 hours, 3 hours, 5 hours, 6 hours, 8 hours, 10 hours, 15 hours, 20 hours, 1 day, 2 days, 3 days, 4 days, 7 days, 10 days, 14 days, 17 days, 20 days, 24 days, 27 days up to a month. In some embodiments, upon administration of the CDP-topoisomerase inhibitor conjugate to a subject, the rate of topoisomerase inhibitor release is dependent primarily upon the rate of hydrolysis as opposed to enzymatic cleavage.

In some embodiments, the CDP-topoisomerase inhibitor conjugate has a molecular weight of 10,000-500,000. In some embodiments, the cyclodextrin moieties make up at least about 2%, 5%, 10%, 20%, 30%, 50% or 80% of the CDP-topoisomerase inhibitor conjugate by weight.

In some embodiments, the CDP-topoisomerase inhibitor conjugate is made by a method comprising providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced. In some embodiments, the cyclodextrin moiety precursors are in a composition, the composition being substantially free of cyclodextrin moieties having other than two positions modified to bear a reactive site (e.g., cyclodextrin moieties having 1, 3, 4, 5, 6, or 7 positions modified to bear a reactive site).

In some embodiments, a comonomer of the CDP-topoisomerase inhibitor conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-topoisomerase inhibitor conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, 1-C(NR₁)—NR₁—, and —B(OR₁)—; and R₁, independently for each occurrence, represents H or a lower alkyl.

In some embodiments, the CDP-topoisomerase inhibitor conjugate is a polymer having attached thereto a plurality of D moieties of the following formula:

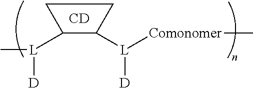

wherein each L is independently a linker, and each D is independently a topoisomerase inhibitor, a prodrug derivative thereof, e.g., a camptothecin or camptothecin derivative, or absent; and each comonomer is independently a comonomer described herein, and n is at least 4, 5, 6, 7, 8, 9, 10, 11, 12, 13, 14, 15, 16, 17, 18, 19 or 20, provided that the polymer comprises at least one topoisomerase inhibitor and in some embodiments, at least two topoisomerase inhibitor moieties. In some embodiments, the molecular weight of the comonomer is from about 2000 to about 5000 Da (e.g., from about 3000 to about 4000 Da (e.g., about 3400 Da).

In some embodiments, the topoisomerase inhibitor is a topoisomerase inhibitor described herein, for example, the topoisomerase inhibitor is a camptothecin or camptothecin derivative described herein. The topoisomerase inhibitor can be attached to the CDP via a functional group such as a hydroxyl group, or where appropriate, an amino group. In some embodiments, one or more of the topoisomerase inhibitor moieties in the CDP-topoisomerase inhibitor conjugate can be replaced with another therapeutic agent, e.g., another anticancer agent or anti-inflammatory agent.

In some embodiments, the CDP-topoisomerase inhibitor conjugate is a polymer having attached thereto a plurality of D moieties of the following formula:

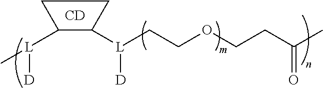

wherein each L is independently a linker, and each D is independently a topoisomerase, a prodrug derivative thereof, e.g., a camptothecin or camptothecin derivative, or absent, provided that the polymer comprises at least one topoisomerase inhibitor and in some embodiments, at least two topoisomerase inhibitor moieties; and

wherein the group

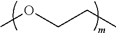

has a Mw of 3.4 kDa or less and n is at least 4, 5, 6, 7, 8, 9, 10, 11, 12, 13, 14, 15, 16, 17, 18, 19 or 20.

In some embodiments, the topoisomerase inhibitor is a topoisomerase inhibitor described herein, for example, the topoisomerase is a camptothecin or camptothecin derivative described herein. The topoisomerase inhibitor can be attached to the CDP via a functional group such as a hydroxyl group, or where appropriate, an amino group. In some embodiments, one or more of the topoisomerase inhibitor moieties in the CDP-topoisomerase inhibitor conjugate can be replaced with another therapeutic agent, e.g., another anticancer agent or anti-inflammatory agent.

In some embodiments, less than all of the L moieties are attached to D moieties, meaning in some embodiments, at least one D is absent. In some embodiments, the loading of the D moieties on the CDP-topoisomerase inhibitor conjugate is from about 1 to about 50% (e.g., from about 1 to about 25%, from about 5 to about 20% or from about 5 to about 15%). In some embodiments, each L independently comprises an amino acid or a derivative thereof. In some embodiments, each L independently comprises a plurality of amino acids or derivatives thereof. In some embodiments, each L is independently a dipeptide or derivative thereof. In one embodiment, L is one or more of: alanine, arginine, histidine, lysine, aspartic acid, glutamic acid, serine, threonine, asparganine, glutamine, cysteine, glycine, proline, isoleucine, leucine, methionine, phenylalanine, tryptophan, tyrosine and valine.

In some embodiments, the CDP-topoisomerase inhibitor conjugate is a polymer having attached thereto a plurality of L-D moieties of the following formula:

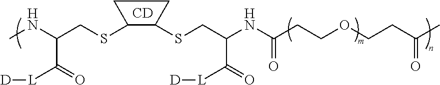

wherein each L is independently a linker or absent, each D is independently a topoisomerase inhibitor, a prodrug derivative thereof, e.g., a camptothecin or camptothecin derivative, or —OH and wherein the group

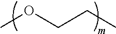

has a Mw of 3.4 kDa or less and n is at least 4, 5, 6, 7, 8, 9, 10, 11, 12, 13, 14, 15, 16, 17, 18, 19 or 20, provided that the polymer comprises at least one topoisomerase inhibitor and in some embodiments, at least two topoisomerase inhibitor moieties.

In some embodiments, less than all of the C(═O) moieties are attached to L-D moieties, meaning in some embodiments, at least one L and/or D is absent. In some embodiments, the loading of the L, D and/or L-D moieties on the CDP-topoisomerase inhibitor conjugate is from about 1 to about 50% (e.g., from about 1 to about 25%, from about 5 to about 20% or from about 5 to about 15%). In some embodiments, each L is independently an amino acid or derivative thereof. In some embodiments, each L is glycine or a derivative thereof.

In some embodiments, one or more of the topoisomerase inhibitor moieties in the CDP-topoisomerase inhibitor conjugate can be replaced with another therapeutic agent, e.g., another anticancer agent or anti-inflammatory agent.

In some embodiments, the CDP-topoisomerase inhibitor conjugate is a polymer having the following formula:

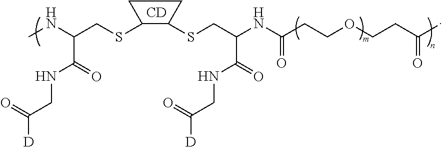

In some embodiments, less than all of the C(═O) moieties are attached to

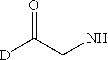

moieties, meaning in some embodiments,

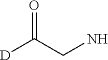

is absent, provided that the polymer comprises at least one topoisomerase inhibitor and in some embodiments, at least two topoisomerase inhibitor moieties. In some embodiments, the loading of the

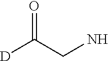

moieties on the CDP-topoisomerase inhibitor conjugate is from about 1 to about 50% (e.g., from about 1 to about 25%, from about 5 to about 20% or from about 5 to about 15%).

In some embodiments, one or more of the topoisomerase inhibitor moieties in the CDP-topoisomerase inhibitor conjugate can be replaced with another therapeutic agent, e.g., another anticancer agent or anti-inflammatory agent.

In some embodiments, the CDP-topoisomerase inhibitor conjugate will contain an topoisomerase inhibitor and at least one additional therapeutic agent. For instance, a topoisomerase inhibitor and one more different cancer drugs, an immunosuppressant, an antibiotic or an anti-inflammatory agent may be grafted on to the polymer via optional linkers. By selecting different linkers for different drugs, the release of each drug may be attenuated to achieve maximal dosage and efficacy.

Cyclodextrins

In certain embodiments, the cyclodextrin moieties make up at least about 2%, 5% or 10% by weight, up to 20%, 30%, 50% or even 80% of the CDP by weight. In certain embodiments, the topoisomerase inhibitors, or targeting ligands make up at least about 1%, 5%, 10% or 15%, 20%, 25%, 30% or even 35% of the CDP by weight. Number-average molecular weight (Mₙ) may also vary widely, but generally fall in the range of about 1,000 to about 500,000 Daltons, preferably from about 5000 to about 200,000 Daltons and, even more preferably, from about 10,000 to about 100,000. Most preferably, Mₙ varies between about 12,000 and 65,000 Daltons. In certain other embodiments, Mₙ varies between about 3000 and 150,000 Daltons. Within a given sample of a subject polymer, a wide range of molecular weights may be present. For example, molecules within the sample may have molecular weights that differ by a factor of 2, 5, 10, 20, 50, 100, or more, or that differ from the average molecular weight by a factor of 2, 5, 10, 20, 50, 100, or more. Exemplary cyclodextrin moieties include cyclic structures consisting essentially of from 7 to 9 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

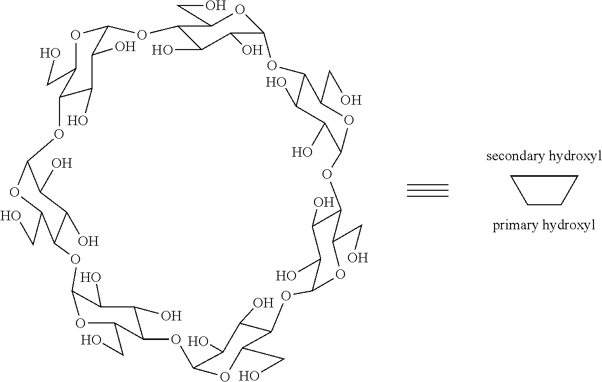

The side on which the secondary hydroxyl groups are located has a wider diameter than the side on which the primary hydroxyl groups are located. The present invention contemplates covalent linkages to cyclodextrin moieties on the primary and/or secondary hydroxyl groups. The hydrophobic nature of the cyclodextrin inner cavity allows for host-guest inclusion complexes of a variety of compounds, e.g., adamantane. (Comprehensive Supramolecular Chemistry, Volume 3, J. L. Atwood et al., eds., Pergamon Press (1996); T. Cserhati, Analytical Biochemistry, 225:328-332 (1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

In certain embodiments, the compounds comprise cyclodextrin moieties and wherein at least one or a plurality of the cyclodextrin moieties of the CDP-topoisomerase inhibitor conjugate is oxidized. In certain embodiments, the cyclodextrin moieties of P alternate with linker moieties in the polymer chain.

Comonomers

In addition to a cyclodextrin moiety, the CDP can also include a comonomer, for example, a comonomer described herein. In some embodiments, a comonomer of the CDP-topoisomerase inhibitor conjugate comprises a moiety selected from the group consisting of: an alkylene chain, polysuccinic anhydride, poly-L-glutamic acid, poly(ethyleneimine), an oligosaccharide, and an amino acid chain. In some embodiments, a CDP-topoisomerase inhibitor conjugate comonomer comprises a polyethylene glycol chain. In some embodiments, a comonomer comprises a moiety selected from: polyglycolic acid and polylactic acid chain. In some embodiments, a comonomer comprises a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁1-C(NR₁)—NR₁—, and —B(OR₁)—; and R₁, independently for each occurrence, represents H or a lower alkyl.

In some embodiments, a comonomer can be and/or can comprise a linker such as a linker described herein.

Exemplary CDP-Topoisomerase Inhibitor Conjugates, Particles and Compositions

In one embodiment, the CDP-topoisomerase inhibitor conjugate forms a particle, e.g., a nanoparticle. The particle can comprise a CDP-topoisomerase inhibitor conjugate, e.g., a plurality of CDP-topoisomerase inhibitor conjugates, e.g., CDP-topoisomerase inhibitor conjugates having the same topoisomerase inhibitor or different topoisomerase inhibitors. The compositions described herein comprise a CDP-topoisomerase inhibitor conjugate or a plurality of CDP-topoisomerase inhibitor conjugates. The composition can also comprise a particle or a plurality of particles described herein.

In one embodiment, the CDP-topoisomerase inhibitor conjugate containing the inclusion complex forms a particle, e.g., a nanoparticle. The nanoparticle ranges in size from 10 to 300 nm in diameter, e.g., 20 to 280, 30 to 250, 40 to 200, 20 to 150, to 100, 20 to 80, 30 to 70, 40 to 60 or 40 to 50 nm diameter. In one embodiment, the particle is 50 to 60 nm, 20 to 60 nm, 30 to 60 nm, 35 to 55 nm, 35 to 50 nm or 35 to 45 nm in diameter.

In one embodiment, the surface charge of the molecule is neutral, or slightly negative. In some embodiments, the zeta potential of the particle surface is from about −80 mV to about 50 mV, about −20 mV to about 20 mV, about −20 mV to about −10 mV, or about −10 mV to about 0.

In some embodiments, the CDP-topoisomerase inhibitor conjugate is a polymer having the following formula C:

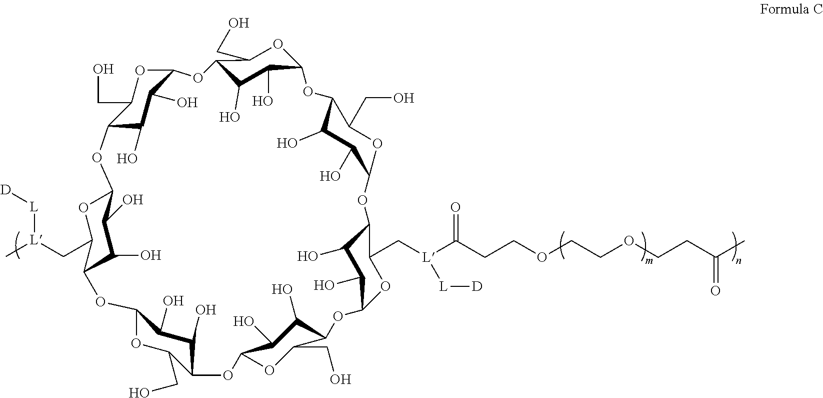

wherein L and L′ independently for each occurrence, is a linker, a bond, or —OH and D, independently for each occurrence, is a topoisomerase inhibitor such as camptothecin (“CPT”), a camptothecin derivative or absent, and

wherein the group

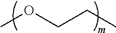

has a Mw of 3.4 kDa or less and n is at least 4, provided that at least one D is CPT or a camptothecin derivative. In some embodiments, at least 2 D moieties are CPT and/or a camptothecin derivative.

In some embodiments, each L′, for each occurrence, is a cysteine. In some embodiments, the cysteine is attached to the cyclodextrin via a sulfide bond. In some embodiments, the cysteine is attached to the PEG containing portion of the polymer via an amide bond.

In some embodiments, the L is a linker (e.g., an amino acid such as glycine). In some embodiments, L is absent. In some embodiments, D-L together form

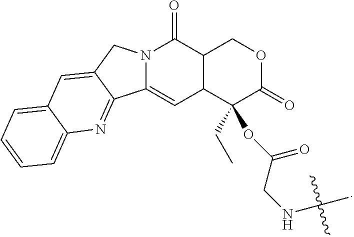

In some embodiments, a plurality of D moieties are absent and at the same position on the polymer, the corresponding L is —OH.

In some embodiments, less than all of the C(═O) moieties of the cysteine residue in the polymer backbone are attached to

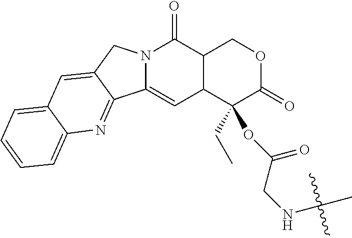 [SMILES: *NCC(=O)O[C@]1(CC)C(=O)OCC2C(=O)N3Cc4cc5ccccc5nc4C3=CC21]

moieties, meaning in some embodiments,

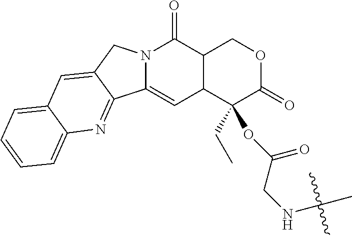 [SMILES: *NCC(=O)O[C@]1(CC)C(=O)OCC2C(=O)N3Cc4cc5ccccc5nc4C3=CC21]

is absent in one or more positions of the polymer backbone, provided that the polymer comprises at least one

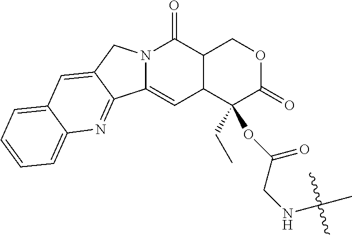 [SMILES: *NCC(=O)O[C@]1(CC)C(=O)OCC2C(=O)N3Cc4cc5ccccc5nc4C3=CC21]

and in some embodiments, at least two

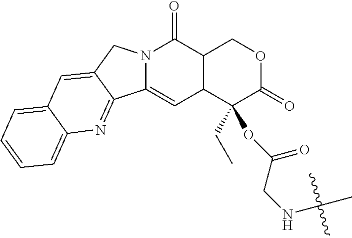 [SMILES: *NCC(=O)O[C@]1(CC)C(=O)OCC2C(=O)N3Cc4cc5ccccc5nc4C3=CC21]

moieties. In some embodiments, the loading of the

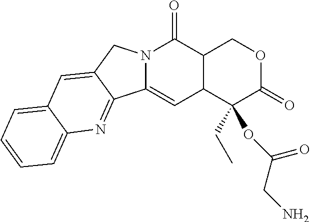 [SMILES: CC[C@@]1(OC(=O)CN)C(=O)OCC2C(=O)N3Cc4cc5ccccc5nc4C3=CC21]

moieties on the CDP-topoisomerase inhibitor conjugate is from about 1 to about 50% (e.g., from about 1 to about 25%, from about 5 to about 20% or from about 5 to about 15%, e.g., from about 6 to about 10%). In some embodiments, the loading of

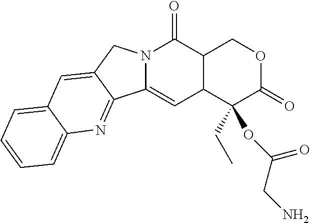 [SMILES: CC[C@@]1(OC(=O)CN)C(=O)OCC2C(=O)N3Cc4cc5ccccc5nc4C3=CC21]

on the CDP is from about 6% to about 10% by weight of the total polymer.

In some embodiments, the CDP-topoisomerase inhibitor conjugate of formula C is a polymer having the following formula:

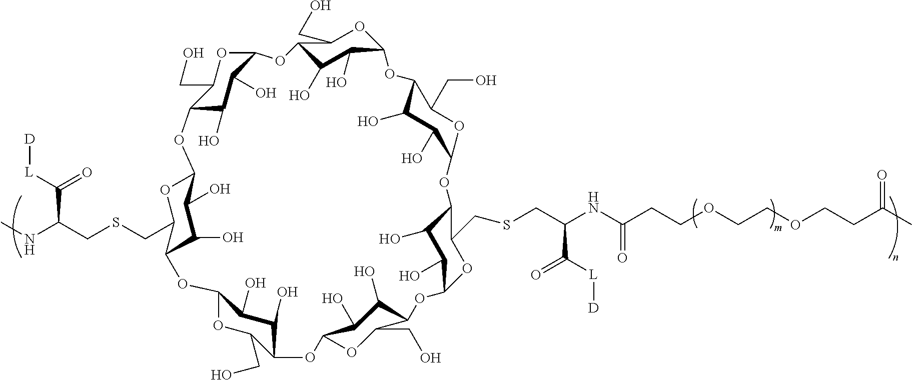

wherein L, independently for each occurrence, is a linker, a bond, or —OH and D, independently for each occurrence, is camptothecin (“CPT”), a camptothecin derivative or absent, and wherein the group

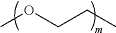

has a Mw of 3.4 kDa or less and n is at least 4, provided that at least one D is CPT or a camptothecin derivative. In some embodiments, at least 2 D moieties are CPT and/or a camptothecin derivative.

In some embodiments, the CDP-camptothecin conjugate of formula C is a polymer of the following formula:

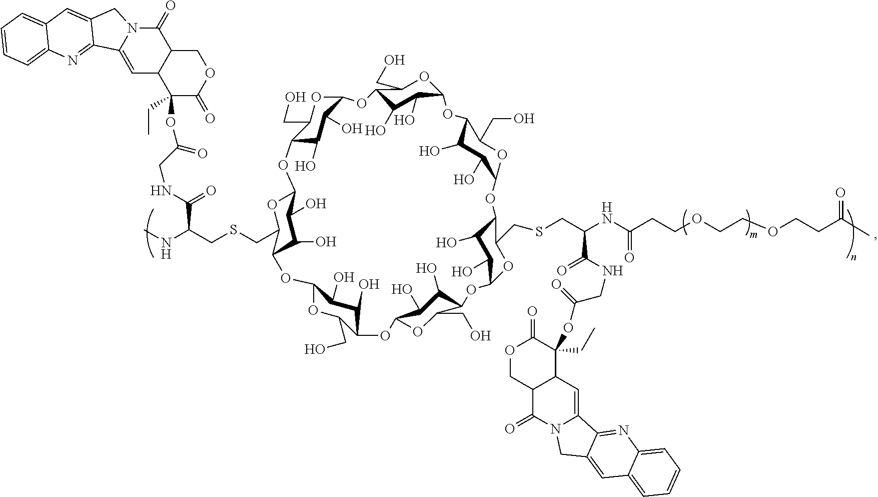

wherein m and n are as defined above, and wherein less than all of the C(═O) sites of the cysteine of the polymer backbone are occupied as indicated above with the CPT-Gly, but instead are free acids, meaning, the theoretical loading of the polymer is less than 100%.

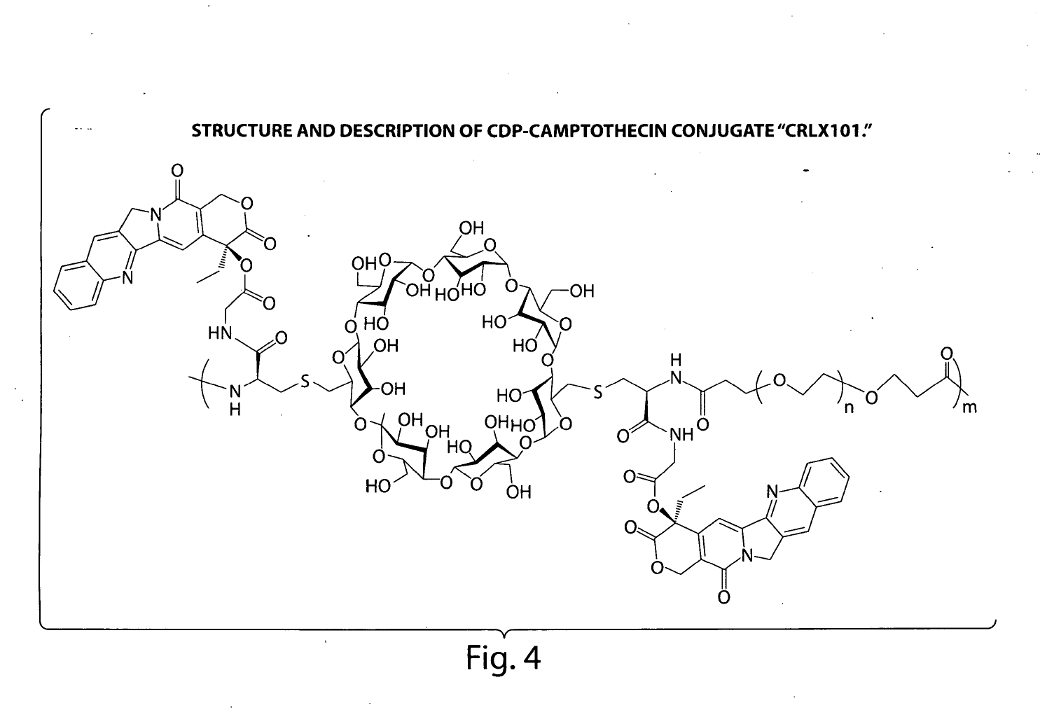

In some embodiments, the CDP-camptothecin conjugate is as provided in FIG. 4, and shown below, which is referred to herein as “CRLX101.”

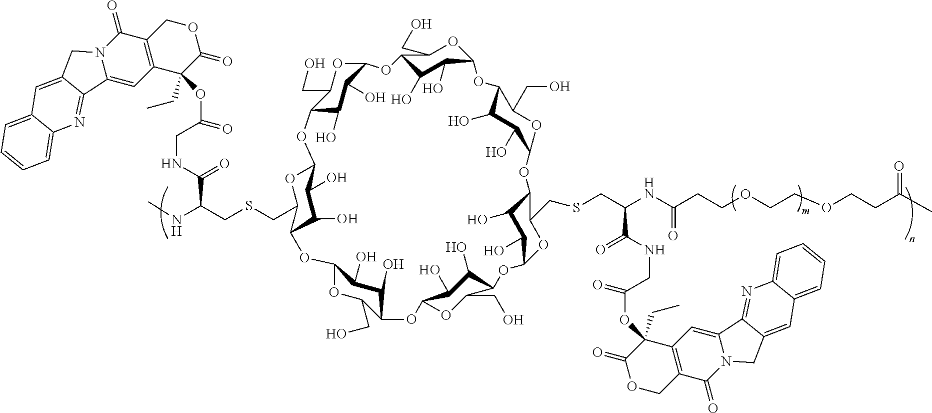

In the above structure:
m=about 77 or the molecular weight of the PEG moiety is 3.4 kDa, e.g., 3.4 kDa+/−10%;
n=is from about 10 to about 18 (e.g., about 14);

the molecular weight of the polymer backbone (i.e., the polymer minus the CPT-gly, which results in the cysteine moieties having a free —C(O)OH) is from about 48 to about 85 kDa;

the polydispersity of the polymer backbone is less than about 2.2; and

the loading of the CPT onto the polymer backbone is from about 6 to about 13% by weight, wherein 13% is theoretical maximum, meaning, in some instances, one or more of the cysteine residues has a free —C(O)OH (i.e., it lacks the CPT-gly).

In some embodiments, the polydispersity of the PEG component in the above structure is less than about 1.1.

In some embodiments, a CDP-camptothecin conjugate described herein has a terminal amine and/or a terminal carboxylic acid.

### Linkers/Tethers

The CDPs described herein can include one or more linkers. In some embodiments, a linker can link a topoisomerase inhibitor to a CDP. In some embodiments, a linker can link camptothecin or a camptothecin derivative to a CDP. In some embodiments, for example, when referring to a linker that links a topoisomerase inhibitor to the CDP, the linker can be referred to as a tether.

In certain embodiments, a plurality of the linker moieties are attached to a topoisomerase inhibitor or prodrug thereof and are cleaved under biological conditions.

Described herein are CDP-topoisomerase inhibitor conjugates comprising a CDP covalently attached to a topoisomerase inhibitor through attachments that are cleaved under biological conditions to release the topoisomerase inhibitor. In certain embodiments, a CDP-topoisomerase inhibitor conjugate comprises a topoisomerase inhibitor covalently attached to a polymer, preferably a biocompatible polymer, through a tether, e.g., a linker, wherein the tether comprises a selectivity-determining moiety and a self-cyclizing moiety which are covalently attached to one another in the tether, e.g., between the polymer and the topoisomerase inhibitor.

In some embodiments, such topoisomerase inhibitors are covalently attached to CDPs through functional groups comprising one or more heteroatoms, for example, hydroxy, thiol, carboxy, amino, and amide groups. Such groups may be covalently attached to the subject polymers through linker groups as described herein, for example, biocleavable linker groups, and/or through tethers, such as a tether comprising a selectivity-determining moiety and a self-cyclizing moiety which are covalently attached to one another.

In certain embodiments, the CDP-topoisomerase inhibitor conjugate comprises a topoisomerase inhibitor covalently attached to the CDP through a tether, wherein the tether comprises a self-cyclizing moiety. In some embodiments, the tether further comprises a selectivity-determining moiety. Thus, one aspect of the invention relates to a polymer conjugate comprising a topoisomerase inhibitor covalently attached to a polymer, preferably a biocompatible polymer, through a tether, wherein the tether comprises a selectivity-determining moiety and a self-cyclizing moiety which are covalently attached to one another.

In some embodiments, the selectivity-determining moiety is bonded to the self-cyclizing moiety between the self-cyclizing moiety and the CDP.

In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, the invention contemplates any combination of the foregoing. Those skilled in the art will recognize that, for example, any topoisomerase inhibitor of the invention in combination with any linker (e.g., self-cyclizing moiety, any selectivity-determining moiety, and/or any topoisomerase inhibitor) are within the scope of the invention.

In certain embodiments, the selectivity-determining moiety is selected such that the bond is cleaved under acidic conditions.

In certain embodiments, where the selectivity-determining moiety is selected such that the bond is cleaved under basic conditions, the selectivity-determining moiety is an aminoalkylcarbonyloxyalkyl moiety. In certain embodiments, the selectivity-determining moiety has a structure

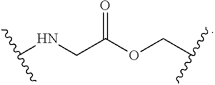 [SMILES: *COC(=O)CN*]

In certain embodiments where the selectivity-determining moiety is selected such that the bond is cleaved enzymatically, it may be selected such that a particular enzyme or class of enzymes cleaves the bond. In certain preferred such embodiments, the selectivity-determining moiety may be selected such that the bond is cleaved by a cathepsin, preferably cathepsin B.

In certain embodiments the selectivity-determining moiety comprises a peptide, preferably a dipeptide, tripeptide, or tetrapeptide. In certain such embodiments, the peptide is a dipeptide is selected from KF and FK, In certain embodiments, the peptide is a tripeptide is selected from GFA, GLA, AVA, GVA, GIA, GVL, GVF, and AVF. In certain embodiments, the peptide is a tetrapeptide selected from GFYA and GFLG, preferably GFLG.

In certain such embodiments, a peptide, such as GFLG, is selected such that the bond between the selectivity-determining moiety and the self-cyclizing moiety is cleaved by a cathepsin, preferably cathepsin B.

In certain embodiments, the selectivity-determining moiety is represented by Formula A:

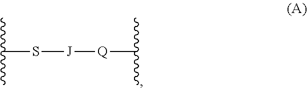

wherein
S a sulfur atom that is part of a disulfide bond;
J is optionally substituted hydrocarbyl; and
Q is O or NR¹³, wherein R¹³ is hydrogen or alkyl.

In certain embodiments, J may be polyethylene glycol, polyethylene, polyester, alkenyl, or alkyl. In certain embodiments, J may represent a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl. In certain embodiments, J may be substituted or unsubstituted lower alkylene, such as ethylene. For example, the selectivity-determining moiety may be

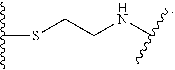 [SMILES: *NCCS*]

In certain embodiments, the selectivity-determining moiety is represented by Formula B:

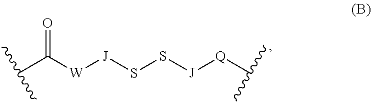

wherein
W is either a direct bond or selected from lower alkyl, NR¹⁴, S, O;
S is sulfur;
J, independently and for each occurrence, is hydrocarbyl or polyethylene glycol;
Q is O or NR¹³, wherein R¹³ is hydrogen or alkyl; and
R¹⁴ is selected from hydrogen and alkyl.

In certain such embodiments, J may be substituted or unsubstituted lower alkyl, such as methylene. In certain such embodiments, J may be an aryl ring. In certain embodiments, the aryl ring is a benzo ring. In certain embodiments W and S are in a 1,2-relationship on the aryl ring. In certain embodiments, the aryl ring may be optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, the aryl ring is optionally substituted with alkyl, alkenyl, alkoxy, aralkyl, aryl, heteroaryl, halogen, —CN, azido, —NR^{x}R^{x}, —CO₂OR^{x}, —C(O)—NR^{x}R^{x}, —C(O)—R^{x}, —NR^{x}—C(O)—R^{x}, —NR^{x}SO₂R^{x}, —SR^{x}, —S(O)R^{x}, —SO₂R^{x}, —SO₂NR^{x}R^{x}, —(C(R^{x})₂)ₙ—OR^{x}, —(C(R^{x})₂)ₙ—NR^{x}R^{x}, and —(C(R^{x})₂)ₙ—SO₂R^{x}; wherein R^{x} is, independently for each occurrence, H or lower alkyl; and n is, independently for each occurrence, an integer from 0 to 2.

In certain embodiments, J, independently and for each occurrence, is polyethylene glycol, polyethylene, polyester, alkenyl, or alkyl.

In certain embodiments, independently and for each occurrence, the linker comprises a hydrocarbylene group comprising one or more methylene groups, wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR³⁰, O or S), —OC(O)—, —C(═O)O, —NR³⁰—, —NR₁CO—, —C(O)NR³⁰—, —S(O)ₙ— (wherein n is 0, 1, or 2), —OC(O)—NR³⁰, —NR³⁰—C(O)—NR³⁰—, —NR³⁰—C(NR³⁰)—NR³⁰—, and —B(OR³⁰)—; and R³⁰, independently for each occurrence, represents H or a lower alkyl.

In certain embodiments, J, independently and for each occurrence, is substituted or unsubstituted lower alkylene. In certain embodiments, J, independently and for each occurrence, is substituted or unsubstituted ethylene.

In certain embodiments, the selectivity-determining moiety is selected from

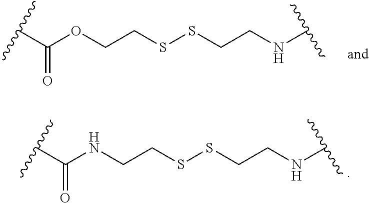 [SMILES: *NCCSSCCOC(*)=O ; *NCCSSCCNC(*)=O]

The selectivity-determining moiety may include groups with bonds that are cleavable under certain conditions, such as disulfide groups. In certain embodiments, the selectivity-determining moiety comprises a disulfide-containing moiety, for example, comprising aryl and/or alkyl group(s) bonded to a disulfide group. In certain embodiments, the selectivity-determining moiety has a structure

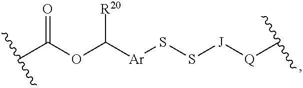

wherein
Ar is a substituted or unsubstituted benzo ring;
J is optionally substituted hydrocarbyl; and

### Q is O or NR¹³,

wherein R¹³ is hydrogen or alkyl.

In certain embodiments, Ar is unsubstituted. In certain embodiments, Ar is a 1,2-benzo ring. For example, suitable moieties within Formula B include:

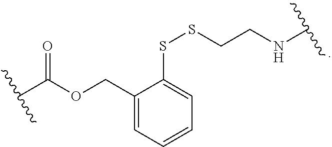 [SMILES: *NCCSSc1ccccc1COC(*)=O]

In certain embodiments, the self-cyclizing moiety is selected such that upon cleavage of the bond between the selectivity-determining moiety and the self-cyclizing moiety, cyclization occurs thereby releasing the therapeutic agent. Such a cleavage-cyclization-release cascade may occur sequentially in discrete steps or substantially simultaneously. Thus, in certain embodiments, there may be a temporal and/or spatial difference between the cleavage and the self-cyclization. The rate of the self-cyclization cascade may depend on pH, e.g., a basic pH may increase the rate of self-cyclization after cleavage. Self-cyclization may have a half-life after introduction in vivo of 24 hours, 18 hours, 14 hours, 10 hours, 6 hours, 3 hours, 2 hours, 1 hour, 30 minutes, 10 minutes, 5 minutes, or 1 minute.

In certain such embodiments, the self-cyclizing moiety may be selected such that, upon cyclization, a five- or six-membered ring is formed, preferably a five-membered ring. In certain such embodiments, the five- or six-membered ring comprises at least one heteroatom selected from oxygen, nitrogen, or sulfur, preferably at least two, wherein the heteroatoms may be the same or different. In certain such embodiments, the heterocyclic ring contains at least one nitrogen, preferably two. In certain such embodiments, the self-cyclizing moiety cyclizes to form an imidazolidone.

In certain embodiments, the self-cyclizing moiety has a structure

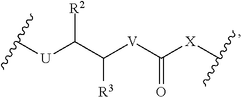

wherein
U is selected from NR¹ and S;
X is selected from O, NR⁵, and S, preferably O or S;
V is selected from O, S and NR⁴, preferably O or NR⁴;
R² and R³ are independently selected from hydrogen, alkyl, and alkoxy; or R² and R³ together with the carbon atoms to which they are attached form a ring; and
R¹, R⁴, and R⁵ are independently selected from hydrogen and alkyl.

In certain embodiments, U is NR¹ and/or V is NR⁴, and R¹ and R⁴ are independently selected from methyl, ethyl, propyl, and isopropyl. In certain embodiments, both R¹ and R⁴ are methyl. On certain embodiments, both R² and R³ are hydrogen. In certain embodiments R² and R³ are independently alkyl, preferably lower alkyl. In certain embodiments, R² and R³ together are —(CH₂)ₙ— wherein n is 3 or 4, thereby forming a cyclopentyl or cyclohexyl ring. In certain embodiments, the nature of R² and R³ may affect the rate of cyclization of the self-cyclizing moiety. In certain such embodiments, it would be expected that the rate of cyclization would be greater when R² and R³ together with the carbon atoms to which they are attached form a ring than the rate when R² and R³ are independently selected from hydrogen, alkyl, and alkoxy. In certain embodiments, U is bonded to the self-cyclizing moiety.

In certain embodiments, the self-cyclizing moiety is selected from

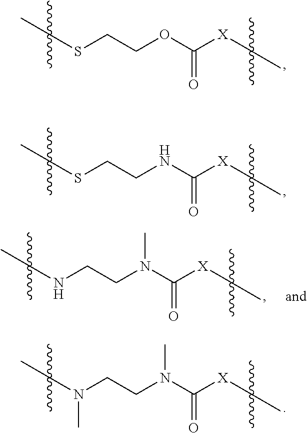

In certain embodiments, the selectivity-determining moiety may connect to the self-cyclizing moiety through carbonyl-heteroatom bonds, e.g., amide, carbamate, carbonate, ester, thioester, and urea bonds.

In certain embodiments, a topoisomerase inhibitor is covalently attached to a polymer through a tether, wherein the tether comprises a selectivity-determining moiety and a self-cyclizing moiety which are covalently attached to one another. In certain embodiments, the self-cyclizing moiety is selected such that after cleavage of the bond between the selectivity-determining moiety and the self-cyclizing moiety, cyclization of the self-cyclizing moiety occurs, thereby releasing the therapeutic agent. As an illustration, ABC may be a selectivity-determining moiety, and DEFGH maybe be a self-cyclizing moiety, and ABC may be selected such that enzyme Y cleaves between C and D. Once cleavage of the bond between C and D progresses to a certain point, D will cyclize onto H, thereby releasing topoisomerase inhibitor X, or a prodrug thereof.

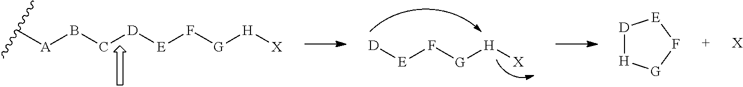

In certain embodiments, topoisomerase inhibitor X may further comprise additional intervening components, including, but not limited to another self-cyclizing moiety or a leaving group linker, such as CO₂ or methoxymethyl, that spontaneously dissociates from the remainder of the molecule after cleavage occurs.

In some embodiments, a linker may be and/or comprise an alkylene chain, a polyethylene glycol (PEG) chain, polysuccinic anhydride, poly-L-glutamic acid, poly(ethyleneimine), an oligosaccharide, an amino acid (e.g., glycine or cysteine), an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, the linker group(s) of the present invention represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —OC(O)—, —C(═O)O, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, the linker group represents a derivatized or non-derivatized amino acid (e.g., glycine or cysteine). In certain embodiments, linker groups with one or more terminal carboxyl groups may be conjugated to the polymer. In certain embodiments, one or more of these terminal carboxyl groups may be capped by covalently attaching them to a therapeutic agent, a targeting moiety, or a cyclodextrin moiety via an (thio)ester or amide bond. In still other embodiments, linker groups with one or more terminal hydroxyl, thiol, or amino groups may be incorporated into the polymer. In preferred embodiments, one or more of these terminal hydroxyl groups may be capped by covalently attaching them to a therapeutic agent, a targeting moiety, or a cyclodextrin moiety via an (thio)ester, amide, carbonate, carbamate, thiocarbonate, or thiocarbamate bond. In certain embodiments, these (thio)ester, amide, (thio)carbonate or (thio)carbamates bonds may be biohydrolyzable, i.e., capable of being hydrolyzed under biological conditions.

In certain embodiments, a linker group represents a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalkyl, or —O—, C(═X) (wherein X is NR₁, O or S), —C(═O)O, —NR₁CO—, —C(O)NR₁—, —S(O)ₙ— (wherein n is 0, 1, or 2), —OC(O)—NR₁, —NR₁—C(O)—NR₁—, —NR₁—C(NR₁)—NR₁—, and —B(OR₁)—; and R₁, independently for each occurrence, represents H or a lower alkyl.

In certain embodiments, a linker group, e.g., between a topoisomerase inhibitor and the CDP, comprises a self-cyclizing moiety. In certain embodiments, a linker group, e.g., between a topoisomerase inhibitor and the CDP, comprises a selectivity-determining moiety.

In certain embodiments as disclosed herein, a linker group, e.g., between a topoisomerase inhibitor and the CDP, comprises a self-cyclizing moiety and a selectivity-determining moiety.

In certain embodiments as disclosed herein, the topoisomerase inhibitor or targeting ligand is covalently bonded to the linker group via a biohydrolyzable bond (e.g., an ester, amide, carbonate, carbamate, or a phosphate).

In certain embodiments as disclosed herein, the CDP comprises cyclodextrin moieties that alternate with linker moieties in the polymer chain.

In certain embodiments, the linker moieties are attached to topoisomerase inhibitors or prodrugs thereof that are cleaved under biological conditions.

In certain embodiments, at least one linker that connects the topoisomerase inhibitor or prodrug thereof to the polymer comprises a group represented by the formula

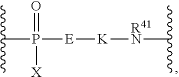

wherein
P is phosphorus;
O is oxygen;
E represents oxygen or NR⁴⁰;
K represents hydrocarbyl;
X is selected from OR⁴² or NR⁴³R⁴⁴; and
R⁴⁰, R⁴¹, R⁴², R⁴³, and R⁴⁴ independently represent hydrogen or optionally substituted alkyl.

In certain embodiments, E is N⁴⁰ and R⁴⁰ is hydrogen.

In certain embodiments, K is lower alkylene (e.g., ethylene).

In certain embodiments, at least one linker comprises a group selected from

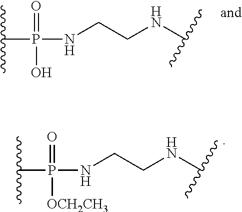 [SMILES: *NCCNP(*)(=O)O ; *NCCNP(*)(=O)OCC]

In certain embodiments, X is OR⁴².

In certain embodiments, the linker group comprises an amino acid or peptide, or derivative thereof (e.g., a glycine or cysteine).

In certain embodiments as disclosed herein, the linker is connected to the topoisomerase inhibitor through a hydroxyl group. In certain embodiments as disclosed herein, the linker is connected to the topoisomerase inhibitor through an amino group.

In certain embodiments, the linker group that connects to the topoisomerase inhibitor may comprise a self-cyclizing moiety, or a selectivity-determining moiety, or both. In certain embodiments, the selectivity-determining moiety is a moiety that promotes selectivity in the cleavage of the bond between the selectivity-determining moiety and the self-cyclizing moiety. Such a moiety may, for example, promote enzymatic cleavage between the selectivity-determining moiety and the self-cyclizing moiety. Alternatively, such a moiety may promote cleavage between the selectivity-determining moiety and the self-cyclizing moiety under acidic conditions or basic conditions.

In certain embodiments, any of the linker groups may comprise a self-cyclizing moiety or a selectivity-determining moiety, or both. In certain embodiments, the selectivity-determining moiety may be bonded to the self-cyclizing moiety between the self-cyclizing moiety and the polymer.

In certain embodiments, any of the linker groups may independently be or include an alkyl chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkyl chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

In certain embodiments, any of the linker groups may independently be an alkyl group wherein one or more methylene groups is optionally replaced by a group Y (provided that none of the Y groups are adjacent to each other), wherein each Y, independently for each occurrence, is selected from aryl, heteroaryl, carbocyclyl, heterocyclyl, or —O—, C(═X) (wherein X is NR¹, O or S), —OC(O)—, —C(═O)O—, —NR¹—, —NR¹CO—, —C(O)NR¹—, —S(O)ₙ— (wherein n is 0, 1, or 2), —OC(O)—NR¹—, —NR¹—C(O)—NR¹—, —NR¹—C(NR¹)—NR¹—, and —B(OR¹)—; and R¹, independently for each occurrence, is H or lower alkyl.

In certain embodiments, the present invention contemplates a CDP, wherein a plurality of topoisomerase inhibitors are covalently attached to the polymer through attachments that are cleaved under biological conditions to release the therapeutic agents as discussed above, wherein administration of the polymer to a subject results in release of the therapeutic agent over a period of at least 2, 3, 5, 6, 8, 10, 15, 20, 24, 36, 48 or even 72 hours.

In some embodiments, the conjugation of the topoisomerase inhibitor to the CDP improves the aqueous solubility of the topoisomerase inhibitor and hence the bioavailability. Accordingly, in one embodiment of the invention, the topoisomerase inhibitor has a log P>0.4, >0.6, >0.8, >1, >2, >3, >4, or even >5.

The CDP-topoisomerase inhibitor conjugate of the present invention preferably has a molecular weight in the range of 10,000 to 500,000; 30,000 to 200,000; or even 70,000 to 150,000 amu.

In certain embodiments, the present invention contemplates attenuating the rate of release of the topoisomerase inhibitor by introducing various tether and/or linking groups between the therapeutic agent and the polymer. Thus, in certain embodiments, the CDP-topoisomerase inhibitor conjugates of the present invention are compositions for controlled delivery of the topoisomerase inhibitor.

CDP-Topoisomerase Inhibitor Conjugate Characteristics

In some embodiments, the CDP and/or CDP-topoisomerase inhibitor conjugate, particle or composition as described herein have polydispersities less than about 3, or even less than about 2.

One embodiment of the present invention provides an improved delivery of certain topoisomerase inhibitor by covalently attaching one or more topoisomerase inhibitors to a CDP. Such conjugation can improve the aqueous solubility and hence the bioavailability of the topoisomerase inhibitor.

The CDP-topoisomerase inhibitor conjugates, particles and compositions described herein preferably have molecular weights in the range of 10,000 to 500,000; 30,000 to 200,000; or even 70,000 to 150,000 amu. In certain embodiments as disclosed herein, the compound has a number average (Mₙ) molecular weight between 1,000 to 500,000 amu, or between 5,000 to 200,000 amu, or between 10,000 to 100,000 amu. One method to determine molecular weight is by gel permeation chromatography (“GPC”), e.g., mixed bed columns, CH₂Cl₂ solvent, light scattering detector, and off-line do/dc. Other methods are known in the art.

In certain embodiments as disclosed herein, the CDP-topoisomerase inhibitor conjugate, particle or composition is biodegradable or bioerodible.

In certain embodiments as disclosed herein, the topoisomerase inhibitor, e.g., the camptothecin, camptothecin derivative, or prodrug thereof makes up at least 3% (e.g., at least about 5%) by weight of the polymer. In certain embodiments, the topoisomerase inhibitor, e.g., the camptothecin, camptothecin derivative or prodrug thereof makes up at least 20% by weight of the compound. In certain embodiments, the topoisomerase inhibitor, e.g., the camptothecin, camptothecin derivative or prodrug thereof makes up at least 5%, 10%, 15%, or at least 20% by weight of the compound.

CDP-topoisomerase inhibitor conjugates, particles and compositions of the present invention may be useful to improve solubility and/or stability of the topoisomerase inhibitor, reduce drug-drug interactions, reduce interactions with blood elements including plasma proteins, reduce or eliminate immunogenicity, protect the topoisomerase inhibitor from metabolism, modulate drug-release kinetics, improve circulation time, improve topoisomerase inhibitor half-life (e.g., in the serum, or in selected tissues, such as tumors), attenuate toxicity, improve efficacy, normalize topoisomerase inhibitor metabolism across subjects of different species, ethnicities, and/or races, and/or provide for targeted delivery into specific cells or tissues.

In other embodiments, the CDP-topoisomerase inhibitor conjugate, particle or composition may be a flexible or flowable material. When the CDP used is itself flowable, the CDP composition of the invention, even when viscous, need not include a biocompatible solvent to be flowable, although trace or residual amounts of biocompatible solvents may still be present.

While it is possible that the biodegradable polymer or the biologically active agent may be dissolved in a small quantity of a solvent that is non-toxic to more efficiently produce an amorphous, monolithic distribution or a fine dispersion of the biologically active agent in the flexible or flowable composition, it is an advantage of the invention that, in a preferred embodiment, no solvent is needed to form a flowable composition. Moreover, the use of solvents is preferably avoided because, once a polymer composition containing solvent is placed totally or partially within the body, the solvent dissipates or diffuses away from the polymer and must be processed and eliminated by the body, placing an extra burden on the body's clearance ability at a time when the illness (and/or other treatments for the illness) may have already deleteriously affected it.

However, when a solvent is used to facilitate mixing or to maintain the flowability of the CDP-topoisomerase inhibitor conjugate, particle or composition, it should be non-toxic, otherwise biocompatible, and should be used in relatively small amounts. Solvents that are toxic should not be used in any material to be placed even partially within a living body. Such a solvent also must not cause substantial tissue irritation or necrosis at the site of administration.

Examples of suitable biocompatible solvents, when used, include N-methyl-2-pyrrolidone, 2-pyrrolidone, ethanol, propylene glycol, acetone, methyl acetate, ethyl acetate, methyl ethyl ketone, dimethylformamide, dimethylsulfoxide, tetrahydrofuran, caprolactam, oleic acid, or 1-dodecylazacylcoheptanone. Preferred solvents include N-methylpyrrolidone, 2-pyrrolidone, dimethylsulfoxide, and acetone because of their solvating ability and their biocompatibility.

In certain embodiments, the CDP-topoisomerase inhibitor conjugates, particles and compositions are soluble in one or more common organic solvents for ease of fabrication and processing. Common organic solvents include such solvents as chloroform, dichloromethane, dichloroethane, 2-butanone, butyl acetate, ethyl butyrate, acetone, ethyl acetate, dimethylacetamide, N-methylpyrrolidone, dimethylformamide, and dimethylsulfoxide.

In certain embodiments, the CDP-topoisomerase inhibitor conjugates, particles and compositions described herein, upon contact with body fluids, undergo gradual degradation. The life of a biodegradable polymer in vivo depends upon, among other things, its molecular weight, crystallinity, biostability, and the degree of crosslinking. In general, the greater the molecular weight, the higher the degree of crystallinity, and the greater the biostability, the slower biodegradation will be.

If a subject composition is formulated with a topoisomerase inhibitor or other material, release of the topoisomerase inhibitor or other material for a sustained or extended period as compared to the release from an isotonic saline solution generally results. Such release profile may result in prolonged delivery (over, say 1 to about 2,000 hours, or alternatively about 2 to about 800 hours) of effective amounts (e.g., about 0.0001 mg/kg/hour to about 10 mg/kg/hour, e.g., 0.001 mg/kg/hour, 0.01 mg/kg/hour, 0.1 mg/kg/hour, 1.0 mg/kg/hour) of the topoisomerase inhibitor or any other material associated with the polymer.

A variety of factors may affect the desired rate of hydrolysis of CDP-topoisomerase inhibitor conjugates, particles and compositions, the desired softness and flexibility of the resulting solid matrix, rate and extent of bioactive material release. Some of such factors include the selection/identity of the various subunits, the enantiomeric or diastereomeric purity of the monomeric subunits, homogeneity of subunits found in the polymer, and the length of the polymer. For instance, the present invention contemplates heteropolymers with varying linkages, and/or the inclusion of other monomeric elements in the polymer, in order to control, for example, the rate of biodegradation of the matrix.

To illustrate further, a wide range of degradation rates may be obtained by adjusting the hydrophobicities of the backbones or side chains of the polymers while still maintaining sufficient biodegradability for the use intended for any such polymer. Such a result may be achieved by varying the various functional groups of the polymer. For example, the combination of a hydrophobic backbone and a hydrophilic linkage produces heterogeneous degradation because cleavage is encouraged whereas water penetration is resisted.

One protocol generally accepted in the field that may be used to determine the release rate of a therapeutic agent such as a topoisomerase inhibitor or other material loaded in the CDP-topoisomerase inhibitor conjugates, particles or compositions of the present invention involves degradation of any such matrix in a 0.1 M PBS solution (pH 7.4) at 37° C., an assay known in the art. For purposes of the present invention, the term “PBS protocol” is used herein to refer to such protocol.

In certain instances, the release rates of different CDP-topoisomerase inhibitor conjugates, particles and compositions of the present invention may be compared by subjecting them to such a protocol. In certain instances, it may be necessary to process polymeric systems in the same fashion to allow direct and relatively accurate comparisons of different systems to be made. For example, the present invention teaches several different methods of formulating the CDP-topoisomerase inhibitor conjugates, particles and compositions. Such comparisons may indicate that any one CDP-topoisomerase inhibitor conjugate, particle or composition releases incorporated material at a rate from about 2 or less to about 1000 or more times faster than another polymeric system.

Alternatively, a comparison may reveal a rate difference of about 3, 5, 7, 10, 25, 50, 100, 250, 500 or 750 times. Even higher rate differences are contemplated by the present invention and release rate protocols.

In certain embodiments, when formulated in a certain manner, the release rate for CDP-topoisomerase inhibitor conjugates, particles and compositions of the present invention may present as mono- or bi-phasic.

Release of any material incorporated into the polymer matrix, which is often provided as a microsphere, may be characterized in certain instances by an initial increased release rate, which may release from about 5 to about 50% or more of any incorporated material, or alternatively about 10, 15, 20, 25, 30 or 40%, followed by a release rate of lesser magnitude.

The release rate of any incorporated material may also be characterized by the amount of such material released per day per mg of polymer matrix. For example, in certain embodiments, the release rate may vary from about 1 ng or less of any incorporated material per day per mg of polymeric system to about 500 or more ng/day/mg. Alternatively, the release rate may be about 0.05, 0.5, 5, 10, 25, 50, 75, 100, 125, 150, 175, 200, 250, 300, 350, 400, 450, or 500 ng/day/mg. In still other embodiments, the release rate of any incorporated material may be 10,000 ng/day/mg, or even higher. In certain instances, materials incorporated and characterized by such release rate protocols may include therapeutic agents, fillers, and other substances.

In another aspect, the rate of release of any material from any CDP-topoisomerase inhibitor conjugate, particle or composition of the present invention may be presented as the half-life of such material in the matrix.

In addition to the embodiment involving protocols for in vitro determination of release rates, in vivo protocols, whereby in certain instances release rates for polymeric systems may be determined in vivo, are also contemplated by the present invention. Other assays useful for determining the release of any material from the polymers of the present system are known in the art.

Physical Structures of the CDP-Topoisomerase Inhibitor Conjugates, Particles and Compositions

The CDP-topoisomerase inhibitor conjugates, particles and compositions may be formed in a variety of shapes. For example, in certain embodiments, CDP-topoisomerase inhibitor conjugates may be presented in the form of microparticles or nanoparticles. Microspheres typically comprise a biodegradable polymer matrix incorporating a drug. Microspheres can be formed by a wide variety of techniques known to those of skill in the art. Examples of microsphere forming techniques include, but are not limited to, (a) phase separation by emulsification and subsequent organic solvent evaporation (including complex emulsion methods such as oil in water emulsions, water in oil emulsions and water-oil-water emulsions); (b) coacervation-phase separation; (c) melt dispersion; (d) interfacial deposition; (e) in situ polymerization; (f) spray drying and spray congealing; (g) air suspension coating; and (h) pan and spray coating. These methods, as well as properties and characteristics of microspheres are disclosed in, for example, U.S. Pat. No. 4,438,253; U.S. Pat. No. 4,652,441; U.S. Pat. No. 5,100,669; U.S. Pat. No. 5,330,768; U.S. Pat. No. 4,526,938; U.S. Pat. No. 5,889,110; U.S. Pat. No. 6,034,175; and European Patent 0258780, the entire disclosures of which are incorporated by reference herein in their entireties.

To prepare microspheres, several methods can be employed depending upon the desired application of the delivery vehicles. Suitable methods include, but are not limited to, spray drying, freeze drying, air drying, vacuum drying, fluidized-bed drying, milling, co-precipitation and critical fluid extraction. In the case of spray drying, freeze drying, air drying, vacuum drying, fluidized-bed drying and critical fluid extraction; the components (stabilizing polyol, bioactive material, buffers, etc.) are first dissolved or suspended in aqueous conditions. In the case of milling, the components are mixed in the dried form and milled by any method known in the art. In the case of co-precipitation, the components are mixed in organic conditions and processed as described below. Spray drying can be used to load the stabilizing polyol with the bioactive material. The components are mixed under aqueous conditions and dried using precision nozzles to produce extremely uniform droplets in a drying chamber. Suitable spray drying machines include, but are not limited to, Buchi, NIRO, APV and Lab-plant spray driers used according to the manufacturer's instructions.

The shape of microparticles and nanoparticles may be determined by scanning electron microscopy. Spherically shaped nanoparticles are used in certain embodiments, for circulation through the bloodstream. If desired, the particles may be fabricated using known techniques into other shapes that are more useful for a specific application.

In addition to intracellular delivery of a topoisomerase inhibitor, it also possible that particles of the CDP-topoisomerase inhibitor conjugates, such as microparticles or nanoparticles, may undergo endocytosis, thereby obtaining access to the cell. The frequency of such an endocytosis process will likely depend on the size of any particle.

In one embodiment, the surface charge of the molecule is neutral, or slightly negative. In some embodiments, the zeta potential of the particle surface is from about −80 mV to about 50 mV.

### CDPs, Methods of Making Same, and Methods of Conjugating CDPs to Topoisomerase Inhibitors

Generally, the CDP-topoisomerase inhibitor conjugates, particles and compositions described herein can be prepared in one of two ways: monomers bearing topoisomerase inhibitors, targeting ligands, and/or cyclodextrin moieties can be polymerized, or polymer backbones can be derivatized with topoisomerase inhibitors, targeting ligands, and/or cyclodextrin moieties. Exemplary methods of making CDPs and CDP-topoisomerase inhibitor conjugates, particles and compositions are described, for example, in U.S. Pat. No. 7,270,808, the contents of which is incorporated herein by reference in its entirety.

The CDPs described herein can be made using a variety of methods including those described herein. In some embodiments, a CDP can be made by: providing cyclodextrin moiety precursors; providing comonomer precursors which do not contain cyclodextrin moieties (comonomer precursors); and copolymerizing the said cyclodextrin moiety precursors and comonomer precursors to thereby make a CDP wherein CDP comprises at least four cyclodextrin moieties and at least four comonomers.

In some embodiments, the at least four cyclodextrin moieties and at least four comonomers alternate in the water soluble linear polymer. In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the cyclodextrin monomers comprise linkers to which the topoisomerase inhibitor may be further linked.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent can be achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring.

In some embodiments, the cyclodextrin moiety comprises an alpha, beta, or gamma cyclodextrin moiety.

In some embodiments, the CDP is suitable for the attachment of sufficient topoisomerase inhibitor such that up to at least 3%, 5%, 10%, 11%, 12%, 13%, 14%, 15%, 20%, 25%, 30%, or even 35% by weight of the CDP, when conjugated, is topoisomerase inhibitor.

In some embodiments, the CDP has a molecular weight of 10,000-500,000 amu. In some embodiments, the cyclodextrin moieties make up at least about 2%, 5%, 10%, 20%, 30%, 50% or 80% of the CDP by weight.

In some embodiments, a CDP of the following formula can be made by the scheme below:

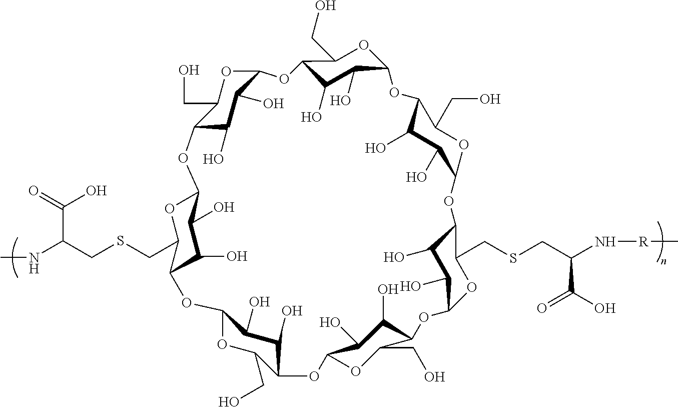

wherein R is of the form:

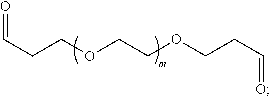

comprising the steps of:

reacting a compound of the formula below:

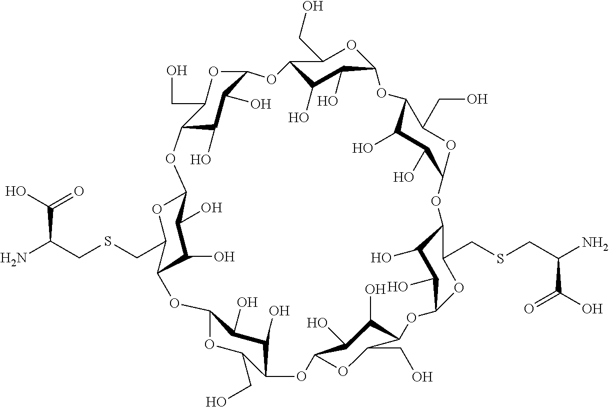 [SMILES: N[C@H](CSCC1O[C@@H]2O[C@@H]3C(CO)O[C@@H](O[C@@H]4C(CO)O[C@@H](O[C@@H]5C(CSC[C@@H](N)C(=O)O)O[C@@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O)C(=O)O]

with a compound of the formula below:

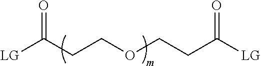

wherein the group

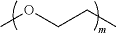

has a Mw of 3.4 kDa or less and n is at least four,

in the presence of a non-nucleophilic organic base in a solvent.

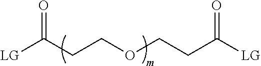

In some embodiments, is

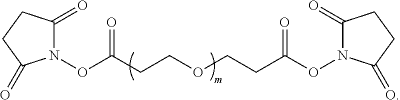

In some embodiments, the solvent is a polar aprotic solvent. In some embodiments, the solvent is DMSO.

In some embodiments, the method also includes the steps of dialysis; and lyophylization.

In some embodiments, a CDP provided below can be made by the following scheme:

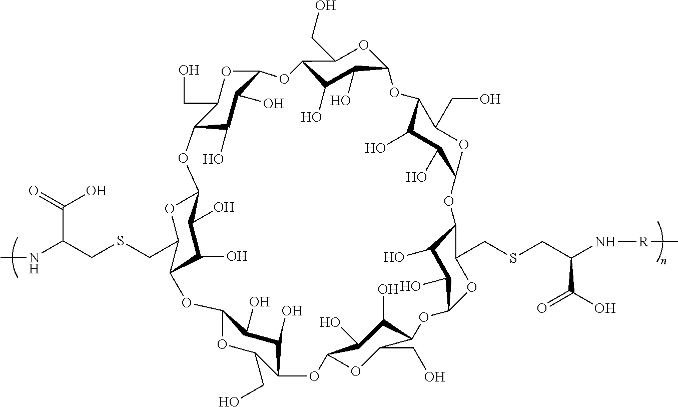

wherein R is of the form:

with a compound provided below:

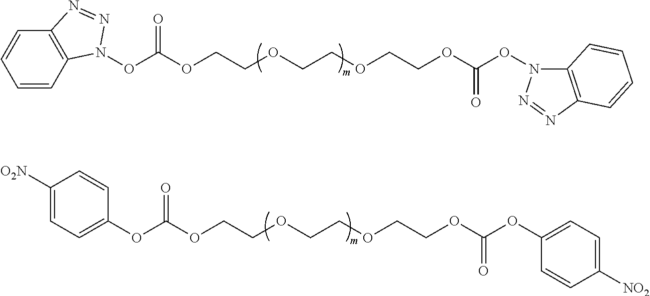

wherein the group

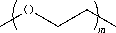

has a Mw of 3.4 kDa;
in the presence of a non-nucleophilic organic base in DMSO;

and dialyzing and lyophilizing the following polymer

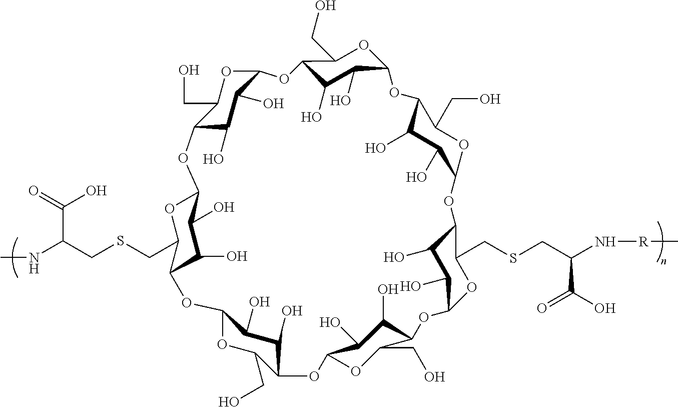

The present invention further contemplates CDPs and CDP-conjugates synthesized using CD-biscysteine monomer and a di-NHS ester such as PEG-DiSPA or PEG-BTC as shown in Scheme I.

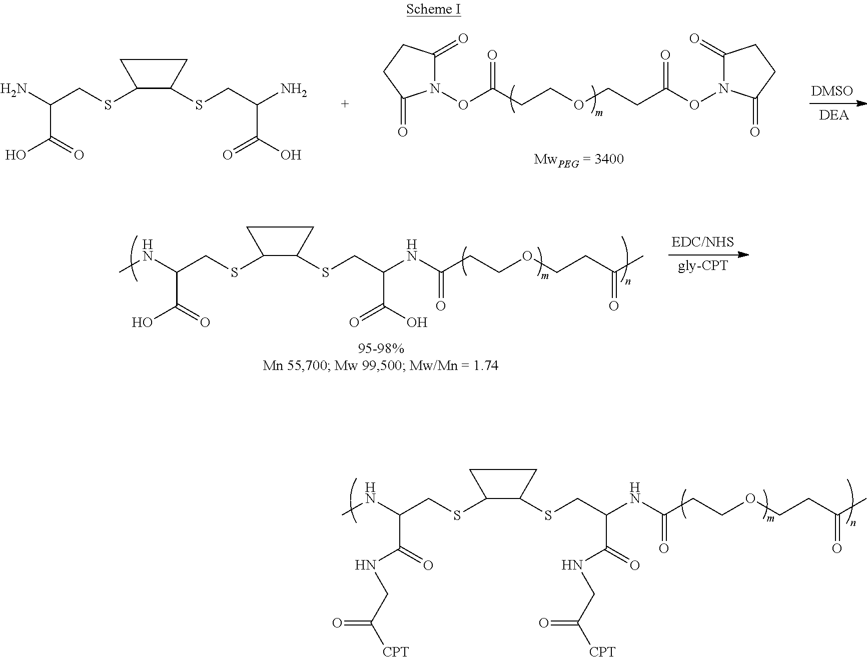

Scheme XIII, as provided above, includes embodiments where gly-CPT is absent in one or more positions as provided above. This can be achieved, for example, when less than 100% yield is achieved when coupling the CPT to the polymer and/or when less than an equivalent amount of CPT is used in the reaction. Accordingly, the loading of the topoisomerase inhibitor such as camptothecin, by weight of the polymer, can vary. Therefore, while Scheme XIII depicts CPT at each cysteine residue of each polymer subunit, the CDP-CPT conjugate can have less than 2 CPT molecules attached to any given polymer subunit of the CDP. For example, in one embodiment, the CDP-CPT conjugate includes several polymer subunits and each of the polymer subunits can independently include two, one or no CPT attached at each cysteine residue of the polymer subunit. In addition, the particles and compositions can include CDP-CPT conjugates having two, one or no CPT attached at each cysteine residue of each polymer subunit of the CDP-CPT conjugate and the conjugates include a mixture of CDP-CPT conjugates that can vary as to the number of CPTs attached to the gly at each of the polymer subunits of the conjugates in the particle or composition.

In some embodiments, a CDP-topoisomerase inhibitor conjugate can be made by providing a CDP comprising cyclodextrin moieties and comonomers which do not contain cyclodextrin moieties (comonomers), wherein the cyclodextrin moieties and comonomers alternate in the CDP and wherein the CDP comprises at least four cyclodextrin moieties and at least four comonomers; and attaching a topoisomerase inhibitor to the CDP.

In some embodiments, one or more of the topoisomerase inhibitor moieties in the CDP-topoisomerase inhibitor conjugate can be replaced with another therapeutic agent, e.g., another anticancer agent or anti-inflammatory agent.

In some embodiments, the topoisomerase inhibitor is attached to the water soluble linear polymer via a linker. In some embodiments, the topoisomerase inhibitor is attached to the water soluble linear polymer through an attachment that is cleaved under biological conditions to release the topoisomerase inhibitor. In some embodiments, the topoisomerase inhibitor is attached to the water soluble linear polymer at a cyclodextrin moiety or a comonomer. In some embodiments, the topoisomerase inhibitor is attached to the water soluble linear polymer via an optional linker to a cyclodextrin moiety or a comonomer.

In some embodiments, the cyclodextrin moieties comprise linkers to which therapeutic agents are linked.

In some embodiments, the CDP is made by a process comprising: providing cyclodextrin moiety precursors, providing comonomer precursors, and copolymerizing said cyclodextrin moiety precursors and comonomer precursors to thereby make a CDP comprising cyclodextrin moieties and comonomers. In some embodiments, the CDP is conjugated with a topoisomerase inhibitor such as camptothecin to provide a CDP-topoisomerase inhibitor conjugate.

In some embodiments, the method includes providing cyclodextrin moiety precursors modified to bear one reactive site at each of exactly two positions, and reacting the cyclodextrin moiety precursors with comonomer precursors having exactly two reactive moieties capable of forming a covalent bond with the reactive sites under polymerization conditions that promote reaction of the reactive sites with the reactive moieties to form covalent bonds between the comonomers and the cyclodextrin moieties, whereby a CDP comprising alternating units of a cyclodextrin moiety and a comonomer is produced.

In some embodiments, the topoisomerase inhibitor is attached to the CDP via a linker. In some embodiments, the linker is cleaved under biological conditions.

In some embodiments, the topoisomerase inhibitor makes up at least 5%, 10%, 11%, 12%, 13%, 14%, 15%, 20%, 25%, 30%, or even 35% by weight of the CDP-topoisomerase inhibitor conjugate.

In some embodiments, the comonomer comprises polyethylene glycol of molecular weight 3,400 Da, the cyclodextrin moiety comprises beta-cyclodextrin, the theoretical maximum loading of camptothecin on a CDP-camptothecin conjugate is 13%, and camptothecin is 6-10% by weight of the CDP-camptothecin conjugate.

In some embodiments, the comonomer precursor is a compound containing at least two functional groups through which reaction and thus linkage of the cyclodextrin moieties is achieved. In some embodiments, the functional groups, which may be the same or different, terminal or internal, of each comonomer precursor comprise an amino, acid, imidazole, hydroxyl, thio, acyl halide, —HC═CH—, —C≡C— group, or derivative thereof. In some embodiments, the two functional groups are the same and are located at termini of the comonomer precursor. In some embodiments, a comonomer contains one or more pendant groups with at least one functional group through which reaction and thus linkage of a therapeutic agent is achieved. In some embodiments, the functional groups, which may be the same or different, terminal or internal, of each comonomer pendant group comprise an amino, acid, imidazole, hydroxyl, thiol, acyl halide, ethylene, ethyne group, or derivative thereof. In some embodiments, the pendant group is a substituted or unsubstituted branched, cyclic or straight chain C1-C10 alkyl, or arylalkyl optionally containing one or more heteroatoms within the chain or ring.

In some embodiments, the cyclodextrin moiety comprises an alpha, beta, or gamma cyclodextrin moiety.

In some embodiments, the topoisomerase inhibitor is poorly soluble in water.

In some embodiments, administration of the CDP-topoisomerase inhibitor conjugate, particle or composition to a subject results in release of the topoisomerase inhibitor over a period of at least 6 hours. In some embodiments, administration of the CDP-topoisomerase inhibitor conjugate, particle or composition to a subject results in release of the topoisomerase inhibitor over a period of 6 hours to a month. In some embodiments, upon administration of the CDP-topoisomerase inhibitor conjugate, particle or composition to a subject the rate of topoisomerase inhibitor release is dependent primarily upon the rate of hydrolysis as opposed to enzymatic cleavage.

In some embodiments, the CDP-topoisomerase inhibitor conjugate, particle or composition has a molecular weight of 10,000-500,000 amu.

In some embodiments, the cyclodextrin moieties make up at least about 2%, 5%, 10%, 20%, 30%, 50% or 80% of the polymer by weight.

In some embodiments, a CDP-polymer conjugate of the following formula can be made as follows:

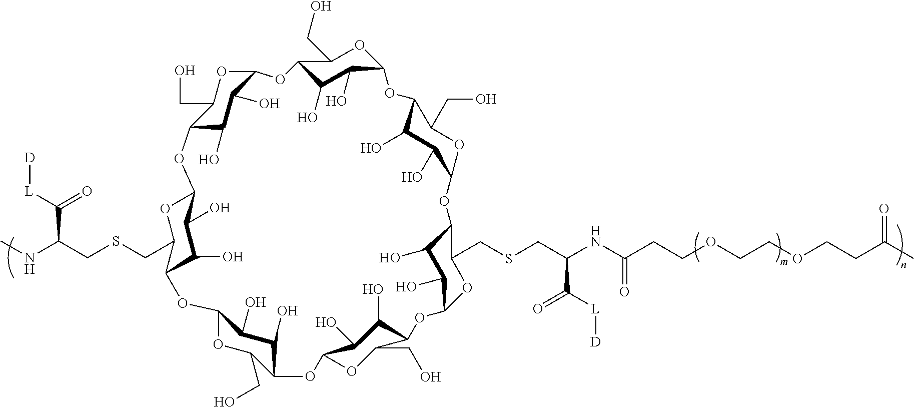

providing a polymer below:

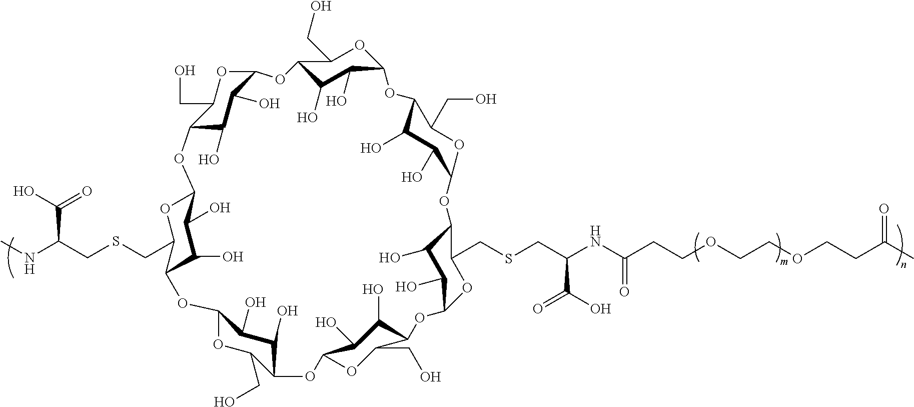

and coupling the polymer with a plurality of L-D moieties, wherein L is a linker, or absent and D is topoisomerase inhibitor such as camptothecin or a camptothecin derivative, to provide:

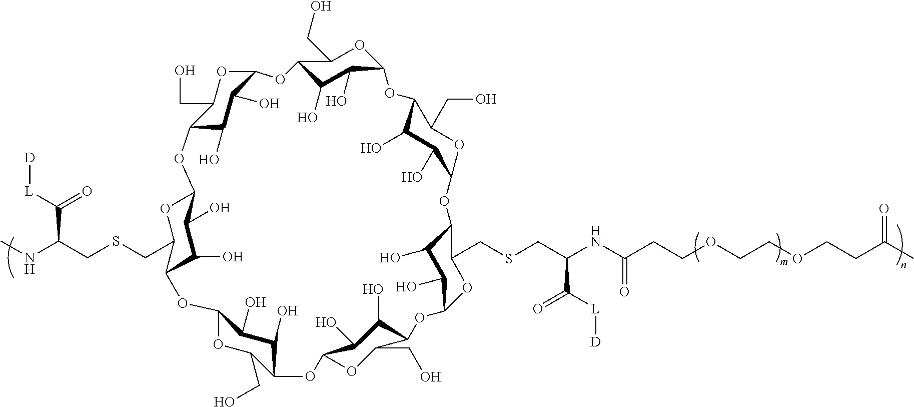

wherein the group

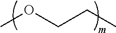

has a Mw of 3.4 kDa or less and n is at least 4, wherein on the final product, L can be a linker, a bond, or OH, and D can be a topoisomerase inhibitor (e.g., camptothecin or a camptothecin derivative) or absent.

In some embodiments, one or more of the topoisomerase inhibitor moieties in the CDP-topoisomerase inhibitor conjugate can be replaced with another therapeutic agent, e.g., another anticancer agent or anti-inflammatory agent.

The reaction scheme as provided above includes embodiments where L-D is absent in one or more positions as provided above. This can be achieved, for example, when less than 100% yield is achieved when coupling the topoisomerase inhibitor-linker to the polymer and/or when less than an equivalent amount of topoisomerase inhibitor-linker is used in the reaction. Accordingly, the loading of the topoisomerase inhibitor, by weight of the polymer, can vary, for example, the loading of the topoisomerase inhibitor can be at least about 3% by weight, e.g., at least about 5%, at least about 8%, at least about 10%, at least about 11%, at least about 12%, at least about 13%, at least about 14%, at least about 15%, or at least about 20%.

In some embodiments, at least a portion of the L moieties of L-D is absent. In some embodiments, each L is independently an amino acid or derivative thereof (e.g., glycine).

In some embodiments, the coupling of the polymer with the plurality of L-D moieties results in the formation of a plurality of amide bonds.

In certain instances, the CDPs are random copolymers, in which the different subunits and/or other monomeric units are distributed randomly throughout the polymer chain. Thus, where the formula Xₘ—Yₙ—Zₒ appears, wherein X, Y and Z are polymer subunits, these subunits may be randomly interspersed throughout the polymer backbone. In part, the term “random” is intended to refer to the situation in which the particular distribution or incorporation of monomeric units in a polymer that has more than one type of monomeric units is not directed or controlled directly by the synthetic protocol, but instead results from features inherent to the polymer system, such as the reactivity, amounts of subunits and other characteristics of the synthetic reaction or other methods of manufacture, processing, or treatment.

### Evaluating Biomarker Expression

In some embodiments, the disclosure features to predictive medicine using biomarker expression as a determinant of efficacy of a cancer treatment, e.g., treatment with a CDP-topoisomerase inhibitor conjugate, particle or composition described herein, and/or the risk of recurrence in a subject having cancer and, e.g., receiving one or more administrations of a CDP-topoisomerase inhibitor conjugate, particle or composition described herein. Contemplated within the scope of this invention is the implementation of biomarker expression analysis as a part of a pharmacogenetic protocol (e.g., using biochips) for evaluating patient status and prognosis. Biomarkers useful in the claimed methods include CAIX, PAI-1, VEGF, gamma-H2AX, RAD51, Topo1, and Topo2.

The level of mRNA corresponding to the biomarker gene in a cell can be determined, e.g., by in situ or in vitro formats.

Biomarker mRNA probes can be used in hybridization or amplification assays that include, but are not limited to, Southern or Northern analyses, polymerase chain reaction analyses and probe arrays. One method for the detection of biomarker levels involves contacting the mRNA with a nucleic acid molecule (probe) that can hybridize to the mRNA encoded by the gene being detected. The nucleic acid probe can be, for example, a full-length nucleic acid or a portion thereof, such as an oligonucleotide of at least 7, 10, 15, 30, 50, 100, 250 or 500 nucleotides in length and sufficient to hybridize under stringent conditions to biomarker mRNA, cDNA, or portions thereof. The probes can be labeled with a detectable reagent to facilitate identification of the probe. Useful reagents include, but are not limited to, radioactivity, fluorescent dyes or enzymes capable of catalyzing a detectable product.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array.

The level of mRNA in a sample that is encoded by a biomarker nucleic acid can be evaluated with nucleic acid amplification, e.g., by RT-PCR (U.S. Pat. No. 4,683,202), ligase chain reaction (Barany, Proc. Natl. Acad. Sci. USA 88:189-193, 1991), self sustained sequence replication (Guatelli et al., Proc. Natl. Acad. Sci. USA 87:1874-1878, 1990), transcriptional amplification system (Kwoh et al., Proc. Natl. Acad. Sci. USA 86:1173-1177, 1989), Q-Beta Replicase (Lizardi et al., Bio/Technology 6:1197, 1988), rolling circle replication (U.S. Pat. No. 5,854,033), or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule including the nucleotide sequence flanked by the primers.

For in situ methods, a cell or tissue sample can be prepared/processed and immobilized on a support, typically a glass slide, and then contacted with a probe that can hybridize to mRNA that encodes the biomarker gene being analyzed.

A variety of methods can be used to determine the level of the biomarker protein. In general, these methods include contacting an agent that selectively binds to the protein, such as an antibody, with a sample to evaluate the level of protein in the sample. In one embodiment, the antibody includes a detectable label. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. The detection methods can be used to detect biomarker protein in a biological sample in vitro as well as in vivo. In vitro techniques for detection of a biomarker protein include enzyme linked immunosorbent assays (ELISAs), immunoprecipitations, immunofluorescence, enzyme immunoassays (EIA), radioimmunoassays (RIA), and Western blot analysis. In vivo techniques for detection of biomarker protein include introducing into a subject a labeled anti-biomarker antibody. For example, the antibody can be labeled with a radioactive marker, e.g., a radioisotope) whose presence and location in a subject can be detected by standard imaging techniques. A radioisotope can be an α-, β-, or γ-emitter. Examples of radioisotopes that can be used include, but are not limited to: yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (.sup. 131I or ¹²⁵I), indium (¹¹¹In), technetium (⁹⁹mTc), phosphorus (³²P), carbon (¹⁴C), and tritium (³H).

### Pharmaceutical Compositions

In another aspect, the present invention provides a composition, e.g., a pharmaceutical composition, comprising a CDP-topoisomerase inhibitor conjugate or particle and a pharmaceutically acceptable carrier or adjuvant.

In some embodiments, a pharmaceutical composition may include a pharmaceutically acceptable salt of a compound described herein, e.g., a CDP-topoisomerase inhibitor conjugate, particle or composition. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a CDP-topoisomerase inhibitor conjugate, particle or composition, which may be any liquid solution compatible with the CDP-topoisomerase inhibitor conjugate, particle or composition, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, and mixtures thereof.

A composition described herein may also include another component, such as an antioxidant, antibacterial, buffer, bulking agent, chelating agent, an inert gas, a tonicity agent and/or a viscosity agent.

In one embodiment, the CDP-topoisomerase inhibitor conjugate, particle or composition is provided in lyophilized form and is reconstituted prior to administration to a subject. The lyophilized CDP-topoisomerase inhibitor conjugate, particle or composition can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the CDP-topoisomerase inhibitor conjugate, particle or composition from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxomers, Tweens, lecithins, saccharides, oligosaccharides, polysaccharides and polyols (e.g., trehalose, mannitol, sorbitol, lactose, sucrose, glucose and dextran), salts and crown ethers. In one embodiment, the lyoprotectant is mannitol.

In some embodiments, the lyophilized CDP-topoisomerase inhibitor conjugate, particle or composition is reconstituted with a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. In some embodiments, the lyophilized CDP-topoisomerase inhibitor conjugate, particle or composition is reconstituted in water for infusion. The lyophilized product and vehicle for reconstitution can be packaged separately in appropriately light-protected vials, e.g., amber or other colored vials. To minimize the amount of surfactant in the reconstituted solution, only a sufficient amount of the vehicle may be provided to form a solution having a concentration of about 2 mg/mL to about 4 mg/mL of the CDP-topoisomerase inhibitor conjugate, particle or composition. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject CDP-topoisomerase inhibitor conjugate, particle or composition with a third vial containing sufficient parenteral diluent to prepare the final concentration for administration. A typical diluent is Lactated Ringer's Injection.

The final dilution of the reconstituted CDP-topoisomerase inhibitor conjugate, particle or composition may be carried out with other preparations having similar utility, for example, 5% Dextrose Injection, Lactated Ringer's and Dextrose for Injection (D5W), Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The dosage form can be, e.g., in a bog, e.g., a bag for infusion or intraperitoneal administration. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

### Routes of Administration

The pharmaceutical compositions described herein may be administered orally, parenterally (e.g., via intravenous, subcutaneous, intracutaneous, intramuscular, intraarticular, intraarterial, intraperitoneal, intrasynovial, intrasternal, intrathecal, intralesional or intracranial injection), topically, mucosally (e.g., rectally or vaginally), nasally, buccally, ophthalmically, via inhalation spray (e.g., delivered via nebulization, propellant or a dry powder device) or via an implanted reservoir. Typically, the compositions are in the form of injectable or infusible solutions. The preferred mode of administration is, e.g., intravenous, subcutaneous, intraperitoneal, intramuscular.

Pharmaceutical compositions suitable for parenteral administration comprise one or more CDP-topoisomerase inhibitor conjugate(s), particle(s) or composition(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the CDP-topoisomerase inhibitor conjugate, particle or composition then depends upon its rate of dissolution which, in turn, may depend upon crystal size and crystalline form. Alternatively, delayed absorption of a parenterally administered drug form is accomplished by dissolving or suspending the CDP-topoisomerase inhibitor conjugate, particle or composition in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the CDP-topoisomerase inhibitor conjugate, particle or composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the CDP-topoisomerase inhibitor conjugate, particle or composition may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more CDP-topoisomerase inhibitor conjugate, particle or composition described herein with one or more suitable non-irritating excipients which is solid at room temperature, but liquid at body temperature. The composition will therefore melt in the rectum or vaginal cavity and release the CDP-topoisomerase inhibitor conjugate, particle or composition. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention.

### Dosages and Dosing Regimens

The CDP-topoisomerase inhibitor conjugate, particle or composition can be formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the subject.

In one embodiment, the CDP-topoisomerase inhibitor conjugate, particle or composition is administered to a subject at a dosage of, e.g., about 1 to 40 mg/m², about 3 to 35 mg/m², about 9 to 40 mg/m², e.g., about 1, 3, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 mg/m² of the topoisomerase inhibitor. Administration can be at regular intervals, such as weekly, or every 2, 3, 4, 5 or 6 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. The CDP-topoisomerase inhibitor conjugate, particle or composition can be administered, e.g., by intravenous or intraperitoneal administration.

In one embodiment, the CDP-topoisomerase inhibitor conjugate, particle or composition is administered as a bolus infusion or intravenous push, e.g., over a period of 15 minutes, 10 minutes, 5 minutes or less. In one embodiment, the CDP-topoisomerase inhibitor conjugate, particle or composition is administered in an amount such the desired dose of the agent is administered. Preferably the dose of the CDP-topoisomerase inhibitor conjugate, particle or composition is a dose described herein.

In one embodiment, the subject receives 1, 2, 3, up to 10 treatments, or more, or until the disorder or a symptom of the disorder is cured, healed, alleviated, relieved, altered, remedied, ameliorated, palliated, improved or affected. For example, the subject receives an infusion once every 1, 2, 3 or 4 weeks until the disorder or a symptom of the disorder is cured, healed, alleviated, relieved, altered, remedied, ameliorated, palliated, improved or affected. Preferably, the dosing schedule is a dosing schedule described herein.

The CDP-topoisomerase inhibitor conjugate, particle or composition can be administered as a first line therapy, e.g., alone or in combination with an additional agent or agents. In other embodiments, a CDP-topoisomerase inhibitor conjugate, particle or composition is administered after a subject has developed resistance to, has failed to respond to or has relapsed after a first line therapy. The CDP-topoisomerase inhibitor conjugate, particle or composition can be administered in combination with a second agent. Preferably, the CDP-topoisomerase inhibitor conjugate, particle or composition is administered in combination with a second agent described herein.

### Kits

A CDP-topoisomerase inhibitor conjugate, particle or composition described herein may be provided in a kit. The kit includes a CDP-topoisomerase inhibitor conjugate, particle or composition described herein and, optionally, a container, a pharmaceutically acceptable carrier and/or informational material. The informational material can be descriptive, instructional, marketing or other material that relates to the methods described herein and/or the use of the CDP-topoisomerase inhibitor conjugate, particle or composition for the methods described herein.

The informational material of the kits is not limited in its form. In one embodiment, the informational material can include information about production of the CDP-topoisomerase inhibitor conjugate, particle or composition, physical properties of the CDP-topoisomerase inhibitor conjugate, particle or composition, concentration, date of expiration, batch or production site information, and so forth. In one embodiment, the informational material relates to methods for administering the CDP-topoisomerase inhibitor conjugate, particle or composition, e.g., by a route of administration described herein and/or at a dose and/or dosing schedule described herein.

In one embodiment, the informational material can include instructions to administer a CDP-topoisomerase inhibitor conjugate, particle or composition described herein in a suitable manner to perform the methods described herein, e.g., in a suitable dose, dosage form, or mode of administration (e.g., a dose, dosage form, or mode of administration described herein). In another embodiment, the informational material can include instructions to administer a CDP-topoisomerase inhibitor conjugate, particle or composition described herein to a suitable subject, e.g., a human, e.g., a human having or at risk for a disorder described herein. In another embodiment, the informational material can include instructions to reconstitute a CDP-topoisomerase inhibitor conjugate, particle or composition described herein into a pharmaceutically acceptable composition.

In one embodiment, the kit includes instructions to use the CDP-topoisomerase inhibitor conjugate, particle or composition, such as for treatment of a subject. The instructions can include methods for reconstituting or diluting the CDP-topoisomerase inhibitor conjugate, particle or composition for use with a particular subject or in combination with a particular chemotherapeutic agent. The instructions can also include methods for reconstituting or diluting the CDP-topoisomerase inhibitor conjugate, particle or composition for use with a particular means of administration, such as by intravenous infusion or intraperitoneal administration.

In another embodiment, the kit includes instructions for treating a subject with a particular indication, such as a particular cancer, or a cancer at a particular stage. For example, the instructions can be for a cancer or cancer at stage described herein, e.g., lung cancer (e.g., non small cell lung cancer and/or small cell lung cancer, e.g., squamous cell non-small cell and/or small cell lung cancer) or ovarian cancer. The instructions may also address first line treatment of a subject who has a particular cancer, or cancer at a stage described herein. The instructions can include, e.g., a dose and/or dosing schedule described herein. The instructions can also address treatment of a subject who has been non-responsive to a first line therapy or has become sensitive (e.g., has one or more unacceptable side effect) to a first line therapy, such as a taxane, an anthracycline, an antimetabolite, a vinca alkaloid, a vascular endothelial growth factor (VEGF) pathway inhibitor, an epidermal growth factor (EGF) pathway inhibitor, an alkylating agent, a platinum-based agent, a vinca alkaloid. In another embodiment, the instructions will describe treatment of selected subjects with the CDP-topoisomerase inhibitor conjugate, particle or composition. For example, the instructions can describe treatment of one or more of: a subject having a cancer that has increased levels of KRAS and/or ST expression, e.g., as compared to a reference standard.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a CDP-topoisomerase inhibitor conjugate, particle or composition described herein and/or its use in the methods described herein. The informational material can also be provided in any combination of formats.

In addition to a CDP-topoisomerase inhibitor conjugate, particle or composition described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a CDP-topoisomerase inhibitor conjugate, particle or composition described herein. In such embodiments, the kit can include instructions for admixing a CDP-topoisomerase inhibitor conjugate, particle or composition described herein and the other ingredients, or for using a CDP-topoisomerase inhibitor conjugate, particle or composition described herein together with the other ingredients. For example, the kit can include an agent, which reduces or inhibits one or more symptom of hypersensitivity, a polysaccharide, and/or an agent which increases urinary excretion and/or neutralizes one or more urinary metabolite.

In another embodiment, the kit includes a second therapeutic agent, such as a second chemotherapeutic agent, e.g., a chemotherapeutic agent or combination of chemotherapeutic agents described herein. In one embodiment, the second agent is in lyophilized or in liquid form. In one embodiment, the CDP-topoisomerase inhibitor conjugate, particle or composition and the second therapeutic agent are in separate containers, and in another embodiment, the CDP-topoisomerase inhibitor conjugate, particle or composition and the second therapeutic agent are packaged in the same container.

In some embodiments, a component of the kit is stored in a sealed vial, e.g., with a rubber or silicone closure (e.g., a polybutadiene or polyisoprene closure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A CDP-topoisomerase inhibitor conjugate, particle or composition described herein can be provided in any form, e.g., liquid, frozen, dried or lyophilized form. It is preferred that a composition including the conjugate, particle or composition, e.g., a composition comprising a particle or particles that include a conjugate described herein be substantially pure and/or sterile. When a CDP-topoisomerase inhibitor conjugate, particle or composition described herein is provided in a liquid solution, the liquid solution preferably is an aqueous solution, with a sterile aqueous solution being preferred. In one embodiment, the CDP-topoisomerase inhibitor conjugate, particle or composition is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a CDP-topoisomerase inhibitor conjugate, particle or composition described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a CDP-topoisomerase inhibitor conjugate, particle or composition described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In one embodiment, the device is a medical implant device, e.g., packaged for surgical insertion.

### Combination Therapy

The CDP-topoisomerase inhibitor conjugate, particle or composition may be used in combination with other known therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The CDP-topoisomerase inhibitor conjugate, particle or composition and the at least one additional therapeutic agent can be administered simultaneously, in the same or in separate compositions, or sequentially. For sequential administration, the CDP-topoisomerase inhibitor conjugate, particle or composition can be administered first, and the additional agent can be administered second, or the order of administration can be reversed.

In some embodiments, the CDP-topoisomerase inhibitor conjugate, particle or composition is administered in combination with a VEGF pathway inhibitor, e.g., aflibercept or bevacizumab.

In some embodiments, the CDP-topoisomerase inhibitor conjugate, particle or composition is administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various monotherapies. The phrase “radiation” includes, but is not limited to, external-beam therapy which involves three dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, the CDP-topoisomerase inhibitor conjugate, particle or composition is administered with at least one additional therapeutic agent, such as a chemotherapeutic agent. In certain embodiments, the CDP-topoisomerase inhibitor conjugate, particle or composition is administered in combination with one or more additional chemotherapeutic agent, e.g., with one or more chemotherapeutic agents described herein. Exemplary classes of chemotherapeutic agents include, e.g., angiogenesis inhibitors which include, without limitation A6 (Angstrom Pharmaceuticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP O2) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharmaceuticals), Combretastatin (Oxigene), CP-751,871 (Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfuram (Antabuse), E7820 (Eisai Limited), E7080 (Eisai Limited), EMD 121974 (Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089 (Foretinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (MedImmune), MLN518 (Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230 (Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

In some embodiment, the CDP-topoisomerase inhibitor conjugate, particle or composition is administered instead of another topoisomerase inhibitor, e.g., instead of a topoisomerase inhibitor as a first line therapy or a second line therapy. For example, the CDP-topoisomerase inhibitor conjugate, particle or composition can be used instead of any of the following topoisomerase inhibitors: a topoisomerase I inhibitor, e.g., camptothecin, irinotecan, SN-38, topotecan, lamellarin D; a topoisomerase II inhibitor, e.g., etoposide, tenoposide, doxorubicin.

In some cases, a hormone and/or steriod can be administered in combination with a CDP-topoisomerase inhibitor conjugate, particle or composition. Examples of hormones and steroids include: 17a-ethinylestradiol (Estinyl®, Ethinoral®, Feminone®, Orestralyn®), diethylstilbestrol (Acnestrol®, Cyren A®, Deladumone®, Diastyl®, Domestrol®, Estrobene®, Estrobene®, Estrosyn®, Fonatol®, Makarol®, Milestrol®, Milestrol®, Neo-Oestronol I®, Oestrogenine®, Oestromenin®, Oestromon®, Palestrol®, Stilbestrol®, Stilbetin®, Stilboestroform®, Stilboestrol®, Synestrin®, Synthoestrin®, Vagestrol®), testosterone (Delatestryl®, Testoderm®, Testolin®, Testostroval®, Testostroval-PA®, Testro AQ®), prednisone (Delta-Dome®, Deltasone®, Liquid Pred®, Lisacort®, Meticorten®, Orasone®, Prednicen-M®, Sk-Prednisone®, Sterapred®), Fluoxymesterone (Android-F®, Halodrin®, Halotestin®, Ora-Testryl®, Ultandren®), dromostanolone propionate (Drolban®, Emdisterone®, Masterid®, Masteril®, Masteron®, Masterone®, Metholone®, Permastril®), testolactone (Teslac®), megestrolacetate (Magestin®, Maygace®, Megace®, Megeron®, Megestat®, Megestil®, Megestin®, Nia®, Niagestin®, Ovaban®, Ovarid®, Volidan®), methylprednisolone (Depo-Medrol®, Medlone 21®, Medrol®, Meprolone®, Metrocort®, Metypred®, Solu-Medrol®, Summicort®), methyl-testosterone (Android®, Testred®, Virilon®), prednisolone (Cortalone®, Delta-Cortef®, Hydeltra®, Hydeltrasol®, Meti-derm®, Prelone®), triamcinolone (Aristocort®), chlorotrianisene (Anisene®, Chlorotrisin®, Clorestrolo®, Clorotrisin®, Hormonisene®, Khlortrianizen®, Merbentul®, Metace®, Rianil®, Tace®, Tace-Fn®, Trianisestrol®), hydroxyprogesterone (Delalutin®, Gestiva™), aminoglutethimide (Cytadren®, Elipten®, Orimeten®), estramustine (Emcyt®), medroxyprogesteroneacetate (Provera®, Depo-Provera®), leuprolide (Lupron®, Viadur®), flutamide (Eulexin®), toremifene (Fareston®), and goserelin (Zoladex®).

In certain embodiments, the CDP-topoisomerase inhibitor conjugate, particle or composition is administered in combination with an anti-microbial (e.g., leptomycin B).

In one embodiment, the CDP-topoisomerase inhibitor conjugate, particle or composition is administered in combination with the agent which ameliorates bladder toxicity associated with therapy, e.g., the agent which increases urinary excretion and/or neutralizes one or more urinary metabolite, is administered prior to, concurrently with and/or after administration with the CDP-topoisomerase inhibitor conjugate, particle or composition. In one embodiment, the agent which ameliorates bladder toxicity associated with therapy is saline, e.g., intravenous saline, D5 half normal saline or D5 water. In one embodiment, the agent which increases urinary excretion and/or neutralizes one or more urinary metabolite is 2-mercaptoethane sulfonate sodium (MESNA). In one embodiment, the agent which ameliorates bladder toxicity associated with therapy is 2-mercaptoethane sulfonate sodium (MESNA) and the MESNA is administered intravenously at a dose of about 10%, 20%, 30% the dose of the camptothecin or camptothecin derivative and/or the MESNA is administered orally at a dose of about 20%, 30%, 40%, 50% the dose of the camptothecin or camptothecin derivative.

In another embodiment, the CDP-topoisomerase inhibitor conjugate, particle or composition is administered in combination with an agent or procedure to mitigate potential side effects from the agent compositions such as cystisis, hypersensitivity, diarrhea, nausea and vomiting.

Cystisis can be mitigated with an agent that increases urinary excretion and/or neutralizes one or more urinary metabolite. For example, cystisis can be mitigated or treated with MESNA.

Diarrhea may be treated with antidiarrheal agents including, but not limited to opioids (e.g., codeine (Codicept®, Coducept®), oxicodeine, percocet, paregoric, tincture of opium, diphenoxylate (Lomotil®), diflenoxin), and loperamide (Imodium A-D®), bismuth subsalicylate, lanreotide, vapreotide (Sanvar®, Sanvar IRC1), motiln antagonists, COX2 inhibitors (e.g., celecoxib (Celebrex®), glutamine (NutreStore®), thalidomide (Synovir®, Thalomid®), traditional antidiarrhea remedies (e.g., kaolin, pectin, berberine and muscarinic agents), octreotide and DPP-IV inhibitors.

DPP-IV inhibitors employed in the present invention are generically and specifically disclosed in PCT Publication Nos.: WO 98/19998, DE 196 16 486 A1, WO 00/34241 and WO 95/15309.

Nausea and vomiting may be treated with antiemetic agents such as dexamethasone (Aeroseb-Dex®, Alba-Dex®, Decaderm®, Decadrol®, Decadron®, Decasone®, Decaspray®, Deenar®, Deronil®, Dex-4®, Dexace®, Dexameth®, Dezone®, Gammacorten®, Hexadrol®, Maxidex®, Sk-Dexamethasone®), metoclopramide (Reglan®), diphenylhydramine (Benadryl®, SK-Diphenhydramine®), lorazepam (Ativan®), ondansetron (Zofran®), prochlorperazine (Bayer A 173®, Buccastem®, Capazine®, Combid®, Compazine®, Compro®, Emelent®, Emetiral®, Eskatrol®, Kronocin®, Meterazin®, Meterazin Maleate®, Meterazine®, Nipodal®, Novamin®, Pasotomin®, Phenotil®, Stemetil®, Stemzine®, Tementil®, Temetid®, Vertigon®), thiethylperazine (Norzine®, Torecan®), and dronabinol (Marinol®).

In some embodiments, the CDP-topoisomerase inhibitor conjugate, particle or composition is administered in combination with an immunosuppressive agent. Immunosuppressive agents suitable for the combination include, but are not limited to natalizumab (Tysabri®), azathioprine (Imuran®), mitoxantrone (Novantrone®), mycophenolate mofetil (Cellcept®), cyclosporins (e.g., Cyclosporin A (Neoral®, Sandimmun®, Sandimmune®, SangCya®), cacineurin inhibitors (e.g., Tacrolimus (Prograf®, Protopic®), sirolimus (Rapamune®), everolimus (Afinitor®), cyclophosphamide (Clafen®, Cytoxan®, Neosar®), or methotrexate (Abitrexate®, Folex®, Methotrexate®, Mexate®)), fingolimod, mycophenolate mofetil (CellCept®), mycophenolic acid (Myfortic®), anti-CD3 antibody, anti-CD25 antibody (e.g., Basiliximab (Simulect®) or daclizumab (Zenapax®)), and anti-TNFα antibody (e.g., Infliximab (Remicade®) or adalimumab (Humira®)).

When employing the methods or compositions, other agents used in the modulation of tumor growth or metastasis in a clinical setting, such as antiemetics, can also be administered as desired.

When formulating the pharmaceutical compositions featured in the invention the clinician may utilize preferred dosages as warranted by the condition of the subject being treated. For example, in one embodiment, a CDP-topoisomerase inhibitor conjugate, particle or composition may be administered at a dosing schedule described herein, e.g., once every one, two, three or four weeks.

Also, in general, a CDP-topoisomerase inhibitor conjugate, particle or composition and an additional chemotherapeutic agent(s) do not have to be administered in the same pharmaceutical composition, and may, because of different physical and chemical characteristics, have to be administered by different routes. For example, the CDP-topoisomerase inhibitor conjugate, particle or composition may be administered intravenously while the chemotherapeutic agent(s) may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

In one embodiment, a CDP-topoisomerase inhibitor conjugate, particle or composition is administered once every three weeks and an additional therapeutic agent (or additional therapeutic agents) may also be administered every three weeks for as long as treatment is required. Examples of other chemotherapeutic agents which are administered one every three weeks include, e.g., a vascular endothelial growth factor (VEGF) pathway inhibitor, e.g., aflibercept.

In another embodiment, the CDP-topoisomerase inhibitor conjugate, particle or composition is administered once every two weeks in combination with one or more additional chemotherapeutic agent that is administered orally. For example, the CDP-topoisomerase inhibitor conjugate, particle or composition can be administered once every two weeks in combination with aflibercept.

The actual dosage of the CDP-topoisomerase inhibitor conjugate, particle or composition and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

The disclosure also encompasses a method for the synergistic treatment of cancer wherein a CDP-topoisomerase inhibitor conjugate, particle or composition is administered in combination with an additional chemotherapeutic agent or agents. For example, the CDP-topoisomerase inhibitor conjugate, particle or composition can be administered in combination with aflibercept.

The particular choice of conjugate, particle or composition and anti-proliferative cytotoxic agent(s) or radiation will depend upon the diagnosis of the attending physicians and their judgment of the condition of the subject and the appropriate treatment protocol.

If the CDP-topoisomerase inhibitor conjugate, particle or composition and the chemotherapeutic agent(s) and/or radiation are not administered simultaneously or essentially simultaneously, then the initial order of administration of the CDP-topoisomerase inhibitor conjugate, particle or composition, and the chemotherapeutic agent(s) and/or radiation, may be varied. Thus, for example, the CDP-topoisomerase inhibitor conjugate, particle or composition may be administered first followed by the administration of the chemotherapeutic agent(s) and/or radiation; or the chemotherapeutic agent(s) and/or radiation may be administered first followed by the administration of the CDP-topoisomerase inhibitor conjugate, particle or composition. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (CDP-topoisomerase inhibitor conjugate, particle or composition, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

### Indications

The disclosed CDP-topoisomerase inhibitor conjugates, particles and compositions are useful in treating proliferative disorders, e.g., treating a tumor, e.g., a primary tumor, and/or metastases thereof, wherein the tumor is a primary tumor or a metastases thereof, e.g., a cancer described herein or a metastases of a cancer described herein.

The methods described herein can be used to treat a solid tumor, a soft tissue tumor or a liquid tumor. Exemplary solid tumors include malignancies (e.g., sarcomas and carcinomas (e.g., adenocarcinoma or squamous cell carcinoma)) of the various organ systems, such as those of brain, lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal cell carcinoma, liver cancer, non-small cell carcinoma of the lung, and cancer of the small intestine. The disclosed methods are also useful in evaluating or treating soft tissue tumors such as those of the tendons, muscles or fat, and liquid tumors.

The methods described herein can be used to treat a cancer that involves or results from bone marrow derived progenitor cell mobilization of one or more of: endothelial progenitor cells (EPCs), hematopoietic progenitor cells (HPCs), immature myeloid cells (iMC, including myeloid derived suppressor cells (MDSC) and mesenchymal progenitor cells (MPC). In a preferred embodiment, the subject has, or is at risk of having, a cancer or metastatic disorder (e.g., a carcinoma). For example, the subject has a primary tumor and has, or is at risk of having, a metastasis of the primary tumor.

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:

Digestive/gastrointestinal cancers such as anal cancer; bile duct cancer (e.g. Klatskin tumor); extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including childhood hepatocellular (liver) cancer; pancreatic cancer including childhood pancreatic cancer; sarcoma, rhabdomyosarcoma; pancreatic cancer, islet cell; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; rhabdomyosarcoma including childhood rhabdomyosarcoma; soft tissue sarcoma including childhood soft tissue sarcoma; clear cell sarcoma of tendon sheaths; and uterine sarcoma;

Breast cancer such as breast cancer and pregnancy including childhood and male breast cancer;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and supratentorial primitive neuroectodermal tumors including childhood and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Hematologic/blood cell cancers such as a leukemia (e.g., acute lymphoblastic leukemia in adults and children; acute myeloid leukemia, e.g., in adults and children; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including Hodgkin's lymphoma in adults and children; Hodgkin's lymphoma during pregnancy; non-Hodgkin's lymphoma including non-Hodgkin's lymphoma in adults and children; non-Hodgkin's lymphoma during pregnancy; mycosis fungoides; Sézary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);

Lung cancer such as non-small cell lung cancer; and small cell lung cancer;

Respiratory cancers such as malignant mesothelioma including malignant mesothelioma in adults and children; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site;

and metastases of the aforementioned cancers can also be treated or prevented in accordance with the methods described herein.

The CDP-topoisomerase inhibitor conjugates, particles and compositions described herein are particularly suited to treat accelerated or metastatic cancers of gastric cancer, colorectal cancer, non-small cell lung cancer, ovarian cancer, and breast cancer. In some embodiments, the cancer is metastatic colorectal cancer (mCRC) that is resistant to or has progressed following treatment with an oxaliplatin-containing regimen.

In some embodiments, the cancer is head or neck cancer.

In one embodiment, a method is provided for a combination treatment of a cancer, such as by treatment with a CDP-topoisomerase inhibitor conjugate, particle or composition and a second therapeutic agent. Various combinations are described herein. The combination can reduce the development of tumors, reduces tumor burden, or produce tumor regression in a mammalian host.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Human Phase 1 Study of CRLX101

The below example describes the first human phase 1 study of CRLX101. The study was composed of two parts. Part 1 of the Phase 1 study had a primary objective to determine the safety profile, toxicity, and pharmacokinetics of CRLX101 when administered weekly for 3 consecutive weeks of every 4-week cycle (the initial dosing regimen, sometimes referred to herein as “weekly×3”). In part 2, after the first twelve patients were enrolled, an every other week schedule was initiated for the remainder of the Phase 1 program (sometimes referred to herein as “biweekly”).

Patients and Methods

Eligibility Criteria.

Patients with histologically or cytologically confirmed metastatic or unresectable solid tumors refractory to standard therapy, or for which no standard curative or palliative therapy existed, were eligible for this trial. Prior treatment with topoisomerase inhibitors was allowed. Main eligibility criteria included male or female patients, at least 18 years of age, with advanced, histologically confirmed solid tumor refractory to standard treatment, or for which no standard therapy existed, measurable or evaluable disease, Eastern Cooperative Oncology Group (ECOG) Performance Status≦2, acceptable organ and bone marrow function, no evidence of clinically significant conduction abnormalities or ischemia, and ejection fraction≧45%. Prior chemotherapy, radiation therapy, or investigational therapy had to be completed within a prescribed interval before enrollment, could not have included high dose chemotherapy with autologous stem cell rescue bone marrow transplantation, and patients could not have been refractory to prior treatment with a topoisomerase I inhibitor.

This trial was conducted at City of Hope (COH) (Duarte, Calif.) following approval of the Clinical Protocol Review and Monitoring Committee (CPRMC) and the Institutional Review Board (IRB). After the initial 18 patients were enrolled, an additional site was added at TGen (Scottsdale, Ariz.) following the approval of WIRB (Western Institutional Review Board).

Study Design and Drug Administration.

This was an open-labeled, single-arm dose-escalation phase 1 study of CRLX101. In part 1, CRLX101 was administered at 6, 12, or 18 mg/m² on a weekly schedule three weeks per month. In part 2, CRLX101 was administered at 12, 15 or 18 mg/m² on an every other week schedule.

In part 1, CRLX101 was administered as an intravenous infusion over 90 minutes on days 1, 8, and 15 followed by a 7 day rest period (28 day cycle). In part 2, CRLX101 was administered as an intravenous infusion over 90 minutes on days 1 and 15, every 28 days. CRLX101 was administered as an intravenous infusion over 60 minutes on days 1 and 15, every 28 days for the last six patients in part 2 of the Phase 1 study.

The starting dose in part 1 was 6 mg/m². Dose escalation was done using an accelerated Simon design (Simon et al., “Accelerated Titration Designs for Phase I Clinical Trials in Oncology” J Natl Cancer Inst. 89:1138-47, 1997) with a modified Fibonacci dose escalation scheme. In brief, patients were accrued in cohorts of one at escalating doses until the occurrence of a dose limiting toxicity (DLT) in the first cycle. Once a DLT occurred, additional patients were accrued in that dosage group to provide for either 3 or 6 patients as provided in a standard dose escalation with the intent of determining the maximum tolerated dose. Concurrent accrual was allowed within the same dose level. No intrapatient dose escalation was permitted.

Toxicity Assessment.

Toxicity was graded according to the National Cancer Institute (NCl) common toxicity criteria (CTCAE) version 3.0. The DLT in a given patient was defined as any treatment-related grade III non-hematologic toxicity, any grade IV hematologic toxicity, or persisting toxicities of any grade requiring delay of scheduled treatment by more than 2 weeks. DLT was based on the first course of treatment.

Rules for Dose Escalation.

One patient was treated at each dose level. If a DLT attributable to the study drug(s) was experienced, up to 5 additional patients were subsequently treated at that dose level. If no additional DLTs were observed at the expanded dose level (i.e., at most 1/6 with an attributable DLT), the dose was escalated. Escalation was terminated when two of six patients experienced any DLT attributable to the study drug at a given dose level. The maximally tolerated dose (MTD) was defined as the dose level preceding the dose at which ≧2/6 patients experienced a first-course DLT. Treatment was continued in an individual patient for a total of 6 cycles at the same dose level if no DLT was observed and if clinical benefit was observed. Therapy was discontinued in any patient if excessive toxicity was experienced. No intra-patient dose escalation was permitted. Patients who completed 6 cycles with clinical benefit had the option of continuing treatment at the same dose level every other week.

Safety and Efficacy Evaluations.

Patients were seen, examined and a complete blood count with differential and serum chemistry was obtained before each dose. Radiographic assessments of tumor response (as evaluated by the Response Evaluation Criteria in Solid Tumor criteria RECIST) were performed every two cycles (Therasse et al., “New Guidelines to Evaluate the Response to Treatment in Solid Tumors. European Organization for Research and Treatment of Cancer, National Cancer Institute of the United States, National Cancer Institute of Canada” J. Natl. Cancer Inst. 92:205-16, 2000).

Treatment Modification.

Patients who experienced a DLT during the initial cycle, or a severe or life threatening non-hematologic toxicity at any time during the study, were dose reduced at the next dose administration when their DLT returned to Grade 1. If a non-hematologic DLT occurred in the interval between dose administrations within a cycle, and the toxicity was not life threatening and resolved rapidly, the next dose administration within the cycle was to be at the next lower dose level. If a hematologic DLT occurred in the interval between dose administrations within a cycle, the next dose administration was to be at the next lower dose level, even if the blood cell counts recovered by the date of the next scheduled administration. Dosing was to be held for any non-hematologic toxicity grade>2, except for grade 2 fatigue and anorexia. After the first cycle, dose modifications were based on interval toxicity, and platelet and granulocyte counts were obtained on the day of treatment.

Hematologic Toxicity:

On day 1 of a cycle, administration of CRLX101 required an absolute granulocyte count (AGC) of ≧1500 and platelet count of ≧100,000. On day 8, if the AGC was ≧1000 and platelet count≧75,000, then CRLX101 was given at full dose and in the same manner for day 15. If the AGC was <1000, the treating physicians could delay the dose of CRLX101 on days 8 and 15. If unacceptable hematologic toxicity persisted >7 days, the CRLX101 dose was reduced to the next lower dose level. Similar standards were used in the biweekly part.

Non-Hematologic Toxicity:

Any grade 2 toxicity that was intolerable to the patients, or any grade 3 or greater non-hematologic toxicity that was attributed to CRLX101, had to return to grade 1 before a new cycle of treatment was started. Any treatment delay>2 weeks because of toxicity due to CRLX101 would result in the patient being removed from the study. If a patient experienced any grade 3 genitourinary (GU) toxicity, the dose of CRLX101 was reduced to the next lower dose level, as CPT has been reported to cause hematuria, cystitis or other GU toxicities, which can be irreversible. If the patient experienced grade 3 non-hematologic toxicities that did not recover to grade 1 before the next treatment, treatment was held until recovery and then reduced by one dose level. If the patient experienced grade 4 non-hematologic toxicities that did not recover to grade 1 before the next treatment, then the dose was reduced by 50% in subsequent cycles. If there were conflicting dose attenuations by hematologic and non-hematologic toxicity, the greater dose reduction was applied. All dose modifications were permanent. Criteria for removal from treatment included disease progression, clinical progression, excessive toxicity, or patient withdrawal.

Plasma/Urine Sampling and Analysis.

Whole blood samples (5 mL) were collected in heparinized tubes at the following times during cycle one: pre-dose, during dosing at 30 minutes, 60 minutes and just prior to end of administration (90 minutes), and then at 0.25, 0.5, 1, 2, 3, 4, 6, 8, 24, (48-72 hr optional), 168 hours (1 week), and 336 hours (only for patients on biweekly schedule) following the first dose. A spot trough PK blood sample was collected prior to dosing on Day 1 and Day 15 of every cycle. Plasma was separated by centrifugation at 1,300×g for 10 minutes at 4° C. Plasma was immediately frozen at −20° C., and stored frozen until analysis. For determination of urinary excretion of polymer conjugated and unbound CPT, total urine collections were performed following dose 1 of the first cycle (0-24 and 24-48 hours post administration. A spot urine sample (15 mL) was collected at 8, 24, 48, 168 (1 wk), and 336 hours (pre-second dose) for more accurate determination of total to free CPT ratio. Once each urine collection was complete, the actual collection times and total volume of urine was recorded and a 5-10 mL aliquot of each was frozen for subsequent drug level analysis.

Samples were extracted and analyzed by LC-MS/MS (liquid chromatography-mass spectrometry/mass spectrometry) using a validated method. In order to determine total CPT in samples, a 20 μL aliquot was incubated with 5 μL 0.2N NaOH for 1 hour to release all CPT from the polymer conjugate. The solution was acidified with 7 μL 44% formic acid. Proteins were precipitated by addition of 160 μL cold (<−20° C.) methanol containing the internal standard 9-nitro camptothecin (9-NC) at a concentration of 8 ng/mL, incubation for 30 minutes on ice, and centrifugation at 14,000 rpm. The resulting supernatant was diluted with an equal volume of 0.5 mM ammonium acetate buffer, pH 3.5, and analyzed by LC-MS/MS.

In order to determine unconjugated CPT in samples, a 20 μL aliquot was acidified with 3 μL 44% formic acid. Proteins were precipitated by addition of 160 μL cold (<−20° C.) methanol containing 9-NC at a concentration of 8 ng/mL, incubation for 30 minutes on ice, and centrifugation at 14,000 rpm. The resulting supernatant was diluted with an equal volume of 0.5 mM ammonium acetate buffer, pH 3.5 and analyzed by LC-MS/MS.

LC-MS/MS was performed on an Agilent 1100 series HPLC system (Palo Alto, Calif.) coupled to a Micromass Quattro Ultima Triple Quadrupole Mass Spectrometer (Micromass, Inc., Beverly, Mass.). HPLC separation was achieved using a Synergy Hydro-RP 4 μm 75×2 mm analytical column (Phenomenex, Torrance, Calif.) preceded by a C₁₈ guard column (Phenomenex, Torrance, Calif.). The isocratic mobile phase consisted of 34% acetonitrile, 66% 0.5 mM ammonium acetate, pH 3.5 at a flow rate of 0.2 mL/min at room temperature. MassLynx version 3.5 software was used for data acquisition and processing.

Immunohistochemical Analysis of Topoisomerase I Expression.

Ascites fluid from one patient with ovarian cancer was obtained pre-treatment and on day 2 post treatment with CRLX101 at 6 mg/m². Cells were pelleted by centrifugation and the pellet frozen. The pellet was formalin fixed and paraffin embedded. Immunohistochemical staining was performed on 5 μm thick sections. Sections were deparaffinized in xylene followed by 100% ethanol. Samples were then quenched in 3% hydrogen peroxide and pretreated to promote antigen retrieval by steam in DIVA/citrate buffer (pH 6.0, Biocare Medical, Concord, Calif.) solution. After antigen retrieval, slides were incubated in Protein Block for 20 minutes. Slides were then incubated with primary antibody overnight at 4° C. Topoisomerase I antibody was rabbit polyclonal from Abcam (Cambridge, Mass.).

The next day, slides were washed in Dako Buffer (DB) and incubated with the appropriate secondary antibodies for 30 minutes at room temperature. After washes in DB, slides were incubated with the chromogen diaminobenzidine tetrahydrochloride (DAB), counterstained with hematoxylin, and mounted.

Topoisomerase I Enzymatic Activity Assay.

Lysates containing total cellular protein were made from the plain frozen ascites cells according to the method of Minagawa et al. (“Enhanced Topoisomerase I Activity and Increased Topoisomerase II Alpha Content in Cisplatin-Resistant Cancer Cell Lines” Jpn J Cancer Res. 88:1218-23, 1997), a procedure used specifically for frozen cells.

The catalytic activity of topoisomerase I was determined by measuring the relaxation of supercoiled plasmid substrate DNA using the Topo I assay kit (TopoGEN, Port Orange, Fla.) following the manufacturer's instructions. Briefly, reaction mixtures consisted of supercoiled plasmid substrate DNA (0.5 μg), whole cell lysate (0.25 μg or 0.5 μg, as indicated in data) and the assay buffer (final concentrations: 10 mM Tris HCl [pH 7.9], 1 mM EDTA, 150 mM NaCl, 0.1% BSA, 0.1 mM spermidine, and 5% glycerol). Reaction mixtures were incubated at 37° C. for 30 minutes, and terminated by adding 5 μL stop buffer/gel loading buffer. Samples were loaded onto a 1% agarose gel in 1×TAE buffer (40 mM Tris base [pH 8.3, adjusted in the 50× stock buffer using glacial acetic acid], 2.5 mM NaOAc, and 0.05 mM EDTA) and electrophoresed at 4-5 volts per centimeter for 3-4 hours. Supercoiled plasmid DNA (0.5 μg) and relaxed DNA (0.5 μg) provided by the Topo I assay kit were used as the control markers. The gel was stained with 0.2 μg/mL ethidium bromide for 20 minutes at room temperature, destained in water for 20 minutes and photographed under ultraviolet (UV) light.

Pharmacokinetic and Statistical Analysis.

Plasma concentration versus time data were analyzed using the ADAPT II (Biomedical Simulations Resource, Los Angeles) non-compartmental model. Results are summarized using descriptive statistics.

Results

Patient Enrollment.

Twenty-four patients were enrolled from June 2006 to April 2010. Patient characteristics are summarized in Table 1. The patients had a variety of solid tumors, and lung cancer was the most common tumor type. All twenty-four patients were considered evaluable for toxicity having received two completed cycles of therapy.

TABLE 1
Patient demographics

| | |
|---|---|
| Total number of patients | 24 |
| Number of evaluable patients | 24 (100%) |
| Median age (range) | 61 (46-79) |
| Gender - Male/Female | 13/11 |
| Ethnicity | |
| Caucasian | 16 (66%) |
| Asian | 6 (25%) |
| Others | 2 (9%) |
| Performance status (ECOG) | |
| 0 | 11 (46%) |
| 1 | 12 (50%) |
| 2 | 1 (4%) |
| Tumor types | |
| Lung | 6 |
| Breast | 3 |
| Urinary/Renal | 3 |
| Liver | 2 |
| Pancreatic | 5 |
| Ovarian | 1 |
| Thyroid | 1 |
| Head and Neck | 1 |
| GI | 1 |
| Endometrial | 1 |
| Prior chemotherapy regimens | |
| 0 | 1 |
| 1 | 5 |
| 2 | 2 |
| 3 | 4 |
| greater than or | 12 |
| equal to 4 | |

Tumor Response.

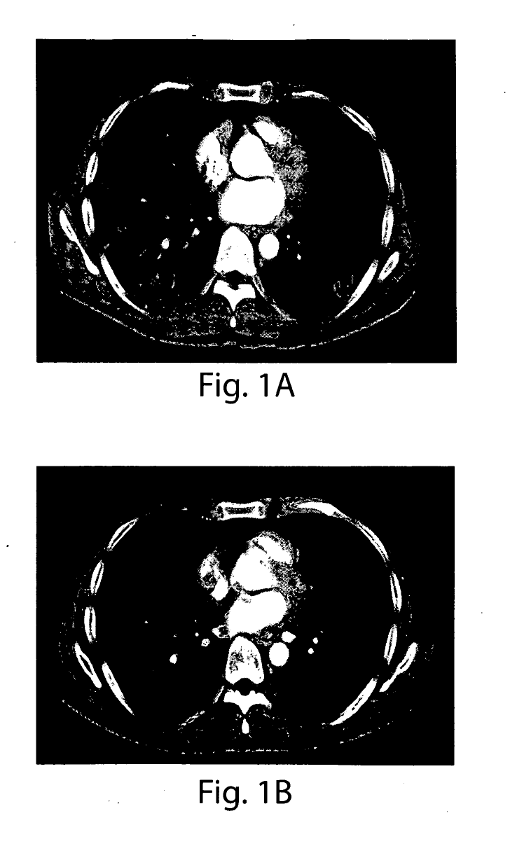

Patient dosing and data are shown in Table 2. At the first dose schedule (part 1), one patient with metastatic pancreatic cancer that had spread to lungs and liver experienced stable disease and received compassionate treatment for a total of 22 cycles before disease progression. The results of a CT scan performed on this patient are shown in FIGS. 1A and 1B. Four patients experienced prolonged (>6 months) SD (stable disease), one patient with renal cancer with lung metastases, two patients with non-small cell lung cancer patient, and one patient with adenocarcinoma of the pancreas. (Table 3). All patients had been heavily pretreated for metastatic disease (see Table 3). Although the protocol allowed for prior treatment with CPT, none of these patients had received prior CPT treatment.

TABLE 2
Analysis of population and patient disposition.

| | | | | | | | Best | |
|---|---|---|---|---|---|---|---|---|
| | | | | | | | Responses | Number of |
| | | Number | Number of | | Pts. w. DR | | During | Pts requiring |
| | Number | of patients | patients | No. Cycles | AE | Discontinuation | therapy (all | dose |
| | of | completed | completed | Median | leading to | AE | eligible | modification |
| | patients. | cycle 1 | cycle 6 | (range) | discontinuation | description | patients) | for toxicity |
| 6 mg/m² | 6 | 6 | 1 (17%) | 2.0 (1-6) | 0 | — | 1 SD (17%) | 0 |
| weekly x3 | | | | | | | | |
| 12 mg/m² | 3 | 3 | 1 (33%) | 2.0 (2-6) | 2 (67%) | Anemia, | 2 SD (67%) | 1 (33%) |
| weekly x3 | | | | | | cystitis | | |
| 18 mg/m² | 3 | 3 | 1 (33%) | 2.0 (1-6) | 2 (67%) | Neutropenia, | 2 SD (67%) | 2 (67%) |
| weekly x3 | | | | | | thrombocytopenia, | | |
| | | | | | | upper | | |
| | | | | | | resp. tract | | |
| | | | | | | infection | | |
| 12 mg/m² | 3 | 3 | 0 | 3.0 (2-4) | 0 | — | — | 0 |
| biweekly | | | | | | | | |
| 15 mg/m² | 6 | 6 | 1 (16%) | 3.0 (2-6) | 1 | Cystitis | 4 SD (67%) | 1 (17%) |
| biweekly | | | | | | | | |
| 18 mg/m² | 3 | 3 | 0 | 2 (1-5) | 1 | Thrombocytopenia | 1 SD (33%) | 2 (67%) |
| biweekly | | | | | | | | |

Note:
One patient still active - presently in cycle 5

Four patients who completed 6 cycles of therapy continued to receive CRLX101 every other week on a compassionate use basis. Abbreviations: AE=adverse event, SD=stable disease, PD=progressive disease, DR=drug related, Pt=patient

TABLE 3
CRLX101 showed activity in this heavily pre-treated patient
population with 4 long-term progression free survivors,
two of which showed minor responses.

| | | | | Duration |
|---|---|---|---|---|
| Tumor | Dose/ | | | of Prior |
| Type | Schedule | Activity | Prior Agents | Response |
| Pancreatic | 6 mg/m² | 22.8 mo PFS, | 5-FU, cisplatin, | 10 mo |
| | weekly x3 | CT scan ↓ 16% | gemcitabine, | |
| | | | radiation | |
| Non-small | 18 mg/m² | 9.7 mo PFS, CT | Getfitinib, | 6 mo |
| cell lung | weekly x3 | scan ↓ 6% | carboplatin, | |
| | | | paclitaxel, | |
| | | | pemetrexed, | |
| | | | vinorelbine, | |
| | | | gemictabine | |
| Renal | 12 mg/m²/ | 7.7 mo PFS, SD | Sunitinib | 18 mo |
| | weekly x3 | | | |
| Non-small | 15 mg/m² | 12.0 mo PFS, | Carboplatin, | 7 mo |
| cell lung | biweekly | SD | paclitaxel, | |
| | | | experimental Rx, | |
| | | | pemetrexed, | |
| | | | gemcitabine, | |
| | | | vinorelbine, | |
| | | | erlotinib | |

Abbreviations:
PFS = progression free survival,
SD = stable disease

Toxicity Evaluation.

All patients in the first twelve enrolled (weekly×3) experienced grade 3 or 4 toxicities at doses above 6 mg/m². The weekly×3 schedule was stopped after these first twelve patients due to the severe toxicity observed in all patients treated at doses higher than 6 mg/m². One patient tolerated 9 mg/m² on a weekly×3 schedule for five cycles (three doses per cycle).

No patients in the second twelve patients enrolled at the bi-weekly schedule experienced severe toxicity until the MTD was reached. Grade 2 DLTs included the following.

One bladder cancer patient experienced grade 2 anemia in cycle 2, day 8. This grade 2 hematologic toxicity resolved in two weeks.

One lung cancer patient experienced elevated amylase in laboratory tests but no clinical manifestation of pancreatitis.

One head and neck cancer patient (nasopharyngeal with heavy previous chemotherapy) experienced delayed onset grade 2 transaminitis in cycle 2, day 1.

One lung cancer patient experienced grade 2 hematologic toxicity which resolved in two weeks.

At the highest dose tested, two patients experienced DLT hematological toxicities. The first patient, a breast cancer patient, who had metastatic disease to lung and other organs and had previously received multiple chemotherapy regimens, developed grade 3 anemia, neutropenia, and thrombocytopenia. This patient required platelet transfusions. The second patient had metastatic lung cancer, and had previously received carboplatin and paclitaxel with a brief response. This patient developed grade 3 neutropenia, requiring dose reduction and granulocyte colony-stimulating factor (G-CSF) support. This hematological toxicity in 2 of 3 patients established 18 mg/m² as the DLT level.

One pancreatic cancer patient experienced grade 2 anemia on cycle 5, day 1.

One patient with hepatocellular cancer passed away one week after cycle 3, day 1 due to progressive disease.

One patient with lung cancer who had received multiple prior regimens, developed grade 2 neutropenia on cycle 3, day 15. This patient required a dose reduction and G-CSF support.

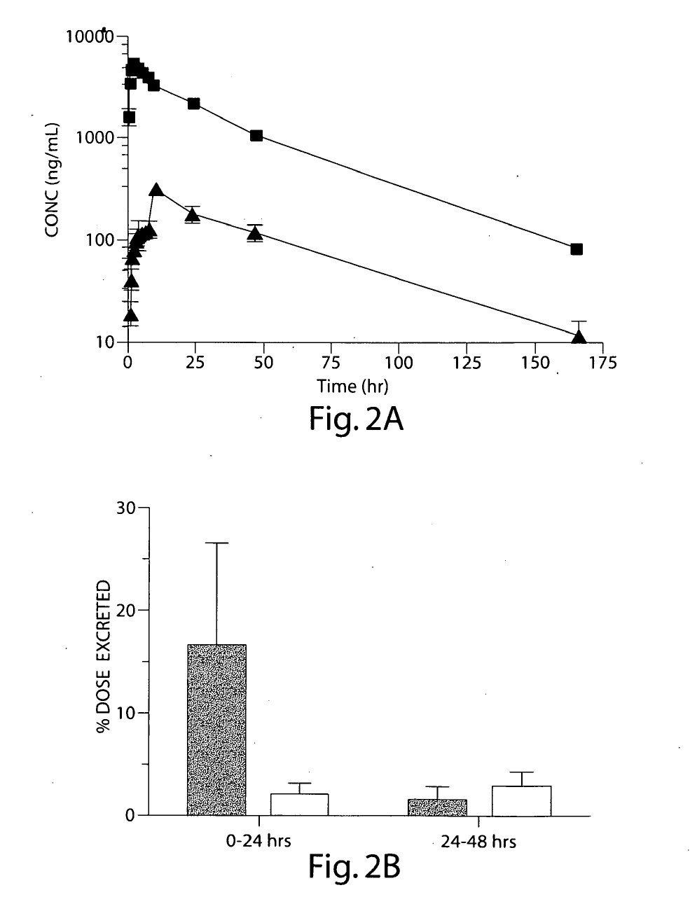

Based on the long terminal half-life of polymer conjugated, and especially unconjugated, CPT (see FIGS. 2A and 2B), the protocol was amended in part 2 to evaluate an every other week schedule. A total of 12 patients were treated on this schedule, three at 12 mg/m² and six at 15 mg/m² and three at 18 mg/m². Except for one occurrence of grade 3 neutropenia, no grade 3/4 hematologic events were recorded at dose level 12 mg/m² and 15 mg/m². At dose level 18 mg/m² one grade 4 leukopenia, two grade 4 neutropenia, two grade 4 thrombocytopenia and one grade 3 anemia were reported under hematological toxicities. This determined the DLT of bone marrow suppression at 18 mg/m² on this schedule. The only other notable non hematologic event was a grade 3 hypersensitivity reaction. (Table 4).

Table 4 shows a summary of all grade 3/4 treatment related toxicities for all evaluable patients. Four patients on the weekly schedule developed delayed onset (after cycle 4) mild hematuria and mild dysuria that may have been related to treatment as previously reported for CPT (Muggia et al., “Phase I Clinical Trial of Weekly and Daily Treatment with Camptothecin (NSC-100880): Correlation with Preclinical Studies” Cancer Chemother Rep 56:515-521, 1972). However, upon evaluation by a urologist, cystitis could not be confirmed. Thus, at 18 mg/m² biweekly two patients were reported who developed DLT, and the MTD for the biweekly schedule was established as 15 mg/m².

TABLE 4
Treatment related grade ¾ hematologic and non-hematologic adverse
events by dose cohort observed during all courses of therapy.

| DOSE | Grade 3 | Grade 4 |
|---|---|---|
| 6 mg/m² | | |
| Elevated CPK | 1 | 0 |
| Fatigue | 1 | |
| lymphopenia | 1 | |
| 12 mg/m² | | |
| hyponatremia | 2 | 0 |
| Dysuria/cystitis | 2 | 0 |
| 18 mg/m² | | |
| thrombocytopenia | 2 | 3 |
| leukopenia | 4 | 1 |
| neutropenia | 4 | 3 |
| anemia | 3 | 0 |
| fatigue | 3 | 0 |
| dehydration | 1 | 0 |
| rash | 1 | 0 |
| SOB | 1 | 0 |
| Hypersensitivity | 1 | 0 |
| reaction | | |

Pharmacokinetic and Toxicokinetic Analysis.

Samples for pharmacokinetic analysis were collected from all patients during cycle 1. Results from this analysis are summarized in FIGS. 2A, 2B and Table 5. The mean elimination half-lives were 31.8±5.7 hr and 43.8±9.7 hr for conjugated and unconjugated CPT, respectively. Volume of distribution of the polymer conjugate was 4.2±1.1 liter and was independent of dose. The low volume of distribution suggests that CRLX101 is initially retained in plasma and avoids rapid first pass clearance. Cₘₐₓ and AUC_{0-inf} were linear across doses and similar when normalized for dose/m². FIGS. 2A and 2B summarize pharmacokinetic parameters measured for polymer conjugated and unconjugated CPT for both schedules.

Unconjugated CPT was slowly released from CRLX101 as shown by increasing plasma concentrations that peaked at 20.2±9.7 hrs. Plasma concentrations of unconjugated CPT were significantly below the plasma concentrations of conjugated CPT at all timepoints, with unconjugated CPT accounting for an average of 8.7±2.7% of total CPT plasma exposure. FIG. 2A shows average plasma time-concentration curves for the biweekly 12 mg/m cohort Systemic plasma clearance of conjugated CPT was 0.12±0.2 L/h, significantly below the kidney and liver blood flows in humans, and was also independent of dose. FIG. 2B shows average urinary excretion of polymer conjugated and unconjugated CPT in the first 48 hours following CRLX101 administration.

Urinary loss of total CPT was variable with an average of 22.8±12.1% of dose excreted during the first 48 hours, of which 78±9% was in the conjugated form. Interestingly, urinary excretion of the polymer conjugate was primarily in the first 24 hours (16.4%±10.0% of dose) compared to the second 24 hours (1.5±1.3% of dose) post administration. Urinary excretion of unconjugated CPT remained approximately constant over both 24 hour periods (2.0±1.1% vs. 2.9±1.4% of dose). Toxicokinetic analysis of the two schedules showed that the predicted monthly exposure for conjugated and unconjugated CPT was similar for 6 mg/m² weekly vs. 12 mg/m² bi-weekly and 12 mg/m² weekly vs. 15 mg/m² bi-weekly; however, fewer patients experienced cycle one drug related adverse events with the bi-weekly regimen.

TABLE 5
Pharmacokinetic parameters and toxicokinetic summary. Values are in
geometric means ± standard deviation.

| | | Cₘₐₓ | | T_{1/2,β} | | AUC | | Per cycle AUC | | # Pt with |
|---|---|---|---|---|---|---|---|---|---|---|
| | | mg/L | | hr | | mg/L/hr | | mg/L/hr | | DR AE ≧ |
| Dose/Schedule | N | Bound | Free | Bound | Free | Bound | Free | Bound | Free | Grade. 3 |
| 6 mg/m² | 6 | 3.55 ± 0.46 | 0.10 ± 0.06 | 31.1 ± 5.2 | 43.7 ± 14.6 | 114.4 ± 21.5 | 11.9 ± 7.0 | 343.2 ± 64.5 | 35.7 ± 21.1 | 2 (33%) |
| weekly x3 | | | | | | | | | | |
| 12 mg/m² | 3 | 5.55 ± 1.33 | 0.18 ± 0.01 | 33.8 ± 5.9 | 61.5 ± 37.6 | 188.5 ± 56.7 | 18.3 ± 3.8 | 565.6 ± 170.1 | 54.9 ± 11.4 | 1 (33%) |
| weekly x3 | | | | | | | | | | |
| 18 mg/m² | 3 | 7.90 ± 1.18 | 0.24 ± 0.06 | 37.7 ± 6.2 | 38.3 ± 4.9 | 248.3 ± 29.2 | 23.7 ± 5.9 | 744.8 ± 87.5 | 71.1 ± 17.8 | 3 (100%) |
| weekly x3 | | | | | | | | | | |
| 12 mg/m² | 3 | 5.56 ± 0.37 | 0.22 ± 0.09 | 27.8 ± 4.3 | 32.5 ± 4.9 | 182.0 ± 21.6 | 14.6 ± 2.5 | 364.0 ± 43.1 | 29.0 ± 5.0 | 0 |
| biweekly | | | | | | | | | | |
| 15 mg/m² | 3 | 8.63 ± 0.76 | 0.27 ± 0.13 | 30.4 ± 1.2 | 48.3 ± 6.5 | 276.7 ± 14.2 | 23.5 ± 9.0 | 553.8 ± 28.3 | 47.0 ± 18.0 | 1 (33%) |
| biweekly | | | | | | | | | | |

Correlative Studies.

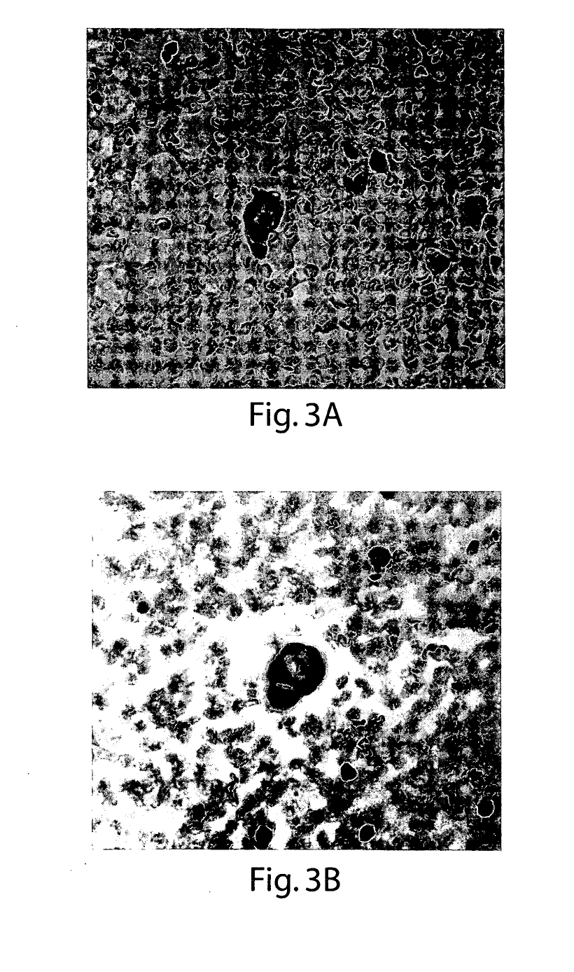

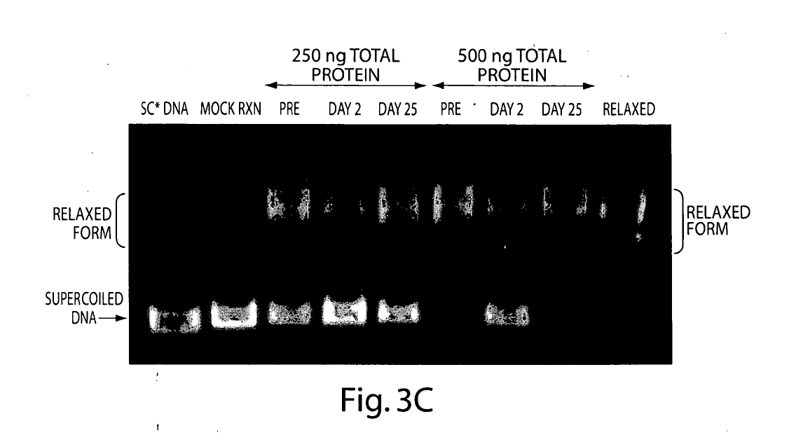

Ascites cells were collected from one patient with ovarian cancer pre-treatment and on Days 2 and 25 post-treatment. Pellets of these cells were frozen for later analysis. Levels of polymer conjugated and unconjugated CPT were also determined in the ascites fluid pre-treatment and on day 2 post-treatment. On day 2, the concentrations detected were 46.6 μg/L for conjugated CPT and 19.6 μg/L for fully active, unconjugated CPT. For each amount of lysate used, there was less topoisomerase I unwinding activity (i.e., more remaining supercoiled DNA) in the Day 2 samples than in the pretreatment or Day 25 samples. This suggests an inhibitory effect of CRLX101 on these cells at this early time point after administration. The ascites cells were also used for immunohistochemistry (1HC) to assess the levels of topoisomerase I. Basic agreement was observed between the topoisomerase I activity assay and topoisomerase I IHC. As seen in FIGS. 3A and 3B, there was a reduction of approximately 30% in staining in the nucleus of ovarian cancer cells isolated from the patient's ascites fluid 2 days after treatment (FIG. 3B), compared to a similar sample taken before drug administration (FIG. 3A). The decrease in topoisomerase I levels directly seen by IHC in these cells at 48 hours explains why much of the supercoiled DNA remains present in lanes of FIG. 3C with reactions from Day 2 lysates; i.e., there is much less enzyme available to act on these substrate molecules.

Summary.

In the phase I trial reported above, two dosing schedules, weekly×3 and every other week (biweekly), were investigated. In the weekly×3 schedule the maximum tolerated dose was approximately 9 mg/m². Hematologic toxicity and cystitis were the DLTs in this schedule. Non-hematologic grade 3/4 adverse events included fatigue in 3 patients (25%), delayed onset hematuria/dysuria in 2 patients (17%), elevated CPK (creatine phosphokinase) in 1 patient (8%), and dehydration in 1 patient (8%), all of which were reversible. Cumulative bladder toxicity in some patients on this schedule was primarily observed post cycle one and may have been related to the long terminal half-life of unconjugated CPT, which is primarily cleared through the kidneys, leading to cumulative bladder irritation. Based on this observation, it was decided to investigate a biweekly schedule as a strategy to reduce cumulative toxicity while maintaining dose delivery.

The biweekly schedule allowed for similar per cycle plasma exposure to be achieved in patients but with a significantly reduced incidence of adverse events. The only observed grade 3/4 adverse event was grade 3 neutropenia in one patient that was reversible. One dose reduction was required on this schedule. On this schedule, CRLX101 was well tolerated without the toxicities normally associated with camptothecin analogs, such as severe diarrhea and hemorrhagic cystitis. The MTD on this schedule was determined to be 15 mg/m².

Pharmacokinetic analysis of CRLX101 was performed after the first dose for all patients. Consistent with preclinical data, the pharmacokinetics of CRLX101 were characterized by a low volume of distribution and limited systemic clearance. Preclinical studies showed accumulation of CRLX101 in tumors and tissues of the reticuloendothelial system such as liver and spleen. Increased release of active CPT from the conjugate was also observed in these tissues. This study also confirmed that release kinetics of CPT were such that plasma levels of unconjugated CPT remained significantly below levels of conjugate at all times. After one week, approximately 10% of the maximum recorded concentration of unconjugated CPT was still detected in plasma, possibly leading to the cumulative toxicity observed on the weekly×3 schedule. On the biweekly schedule however, unconjugated CPT levels dropped below the limit of quantitation before the second dose, which may explain the lack of urinary side effects on this schedule.

In general, CRLX101 was well tolerated and myelosuppression was the DLT. Ten out of twenty-four patients demonstrated stable disease on CT scan evaluation at the end of cycle 2. One pancreatic cancer patient remained stable for 22.8 months. Serum and urine PK data from all the treated patients indicated that the mean elimination half-lives for conjugated and unconjugated CPT were 31.8 hr and 43.8 hr, respectively. Cₘₐₓ and AUC_{0-inf} were linear across doses and similar when normalized for dose/m². The biweekly schedule allowed for similar per cycle plasma exposure to be achieved in patients, but with a significantly reduced incidence of adverse events.

### Example 2

### Synthesis of 6^{A},6^{D}-Bis-(2-amino-2-carboxylethylthio)-6^{A},6^{D}-dideoxy-β-cyclodextrin, 4 (CD-BiSCys)

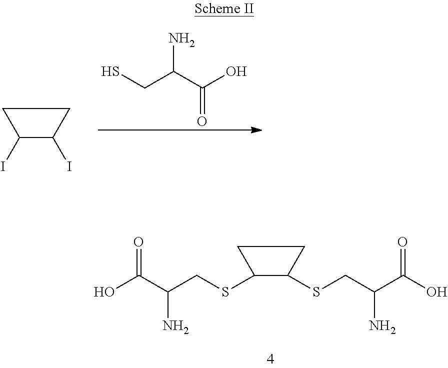 [SMILES: IC1CCC1I ; NC(CS)C(=O)O ; NC(CSC1CCC1SCC(N)C(=O)O)C(=O)O]

167 mL of 0.1 M sodium carbonate buffer were degassed for 45 minutes in a 500 mL 2-neck round bottom flask equipped with a magnetic stir bar, a condenser and septum. To this solution were added 1.96 g (16.2 mmol) of L-cysteine and 10.0 g (73.8 mmol) of diiodo, deoxy-β-cyclodextrin 2. The resulting suspension was heated at a reflux temperature for 4.5 h until the solution turned clear (colorless). The solution was then cooled to room temperature and acidified to pH 3 using 1N HCl. The product was precipitated by slow addition of acetone (3 times weight ratio of the solution). This afforded 9.0 g crude material containing CD-biscysteine (90.0%), unreacted cyclodextrin, CD-mono-cysteine and cystine. The resulting solid was subjected to anionic exchange column chromatography (SuperQ650M, Tosoh Bioscience) using a gradient elution of 0-0.4M ammonium bicarbonate. All fractions were analyzed by HPLC. The desired fractions were combined and the solvent was reduced to 100 mL under vacuum. The final product was either precipitated by adding acetone or by adding methanol (3 times weight ratio of the solution). 4 was obtained in 60-90% yield. ¹H NMR (D₂O) δ 5.08 (m, 7H, CD-2-CH), 3.79-3.94 (m, 30H, CD-3,4-CH, CD-CH₂, Cys-CH), 3.49-3.62 (m, 14H, CD-5,6-CH), 2.92-3.30 (m, 4H, Cys-CH₂). ¹³C NMR (D₂O) δ 172.3, 101.9, 83.9, 81.6, 81.5, 73.3, 72.2, 72.0, 60.7, 54.0, 34.0, 30.6. ESI/MS (m/z): 1342 [M]⁺, 1364 [M+Na]⁺. Purity of 4 was confirmed by HPLC.

### Example 3

### Synthesis of Gly-CPT (Structure 11) (Greenwald et al., Bioorg. Med. Chem., 1998, 6, 551-562)

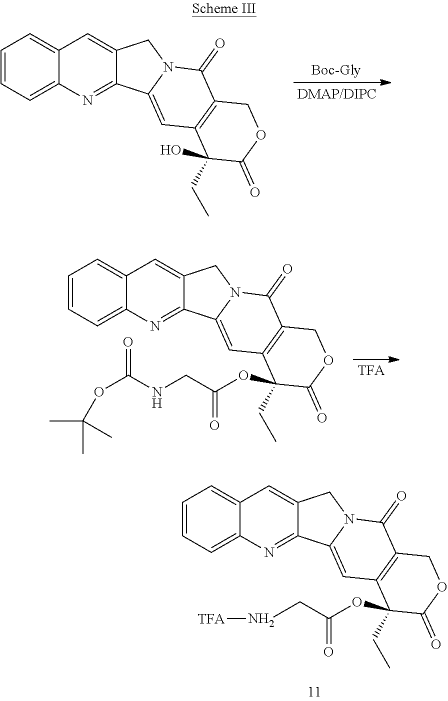

t-Boc-glycine (0.9 g, 4.7 mmol) was dissolved in 350 mL of anhydrous methylene chloride at room temperature, and to this solution were added DIPC (0.75 mL, 4.7 mmol), DMAP (382 mg, 3.13 mmol) and camptothecin (0.55 g, 1.57 mmol) at 0° C. The reaction mixture was allowed to warm to room temperature and left for 16 h. The solution was washed with 0.1 N HCl, dried and evaporated under reduced pressure to yield a white solid, which was recrystallized from methanol to give camptothecin-20-ester of t-Boc-glycine: ¹H NMR (DMSO-d₆) 7.5-8.8 (m), 7.3 (s), 5.5 (s), 5.3 (s), 4 (m), 2.1 (m), 1.6 (s), 1.3 (d), 0.9 (t). Camptothecin-20-ester of t-Boc-glycine (0.595 g, 1.06 mmol) was dissolved in a mixture of methylene chloride (7.5 mL) and TFA (7.5 mL) and stirred at room temperature for 1 h. Solvent was removed and the residue was recrystallized from methylene chloride and ether to give 0.45 g of 11. ¹H NMR (DMSO-d₆) δ7.7-8.5 (m); 7.2 (s), 5.6 (s), 5.4 (s), 4.4 (m), 2.2 (m), 1.6 (d), 1.0 (t), ¹³C NMR (DMSO-d₆) δ168.6, 166.6, 156.5, 152.2, 147.9, 146.2, 144.3, 131.9, 130.6, 129.7, 128.8, 128.6, 128.0, 127.8, 119.0, 95.0, 77.6, 66.6, 50.5, 47.9, 30.2, 15.9, 7.9. ESI/MS (m/z) expected 405. Found 406 (M+H).

### Example 4

### Synthesis and Characterization of CD-BisCys-Peg3400 Copolymers 36 and their CPT Conjugates 37

### A. Synthesis and Characterization of CD-BisCys-Peg3400 Copolymers 36

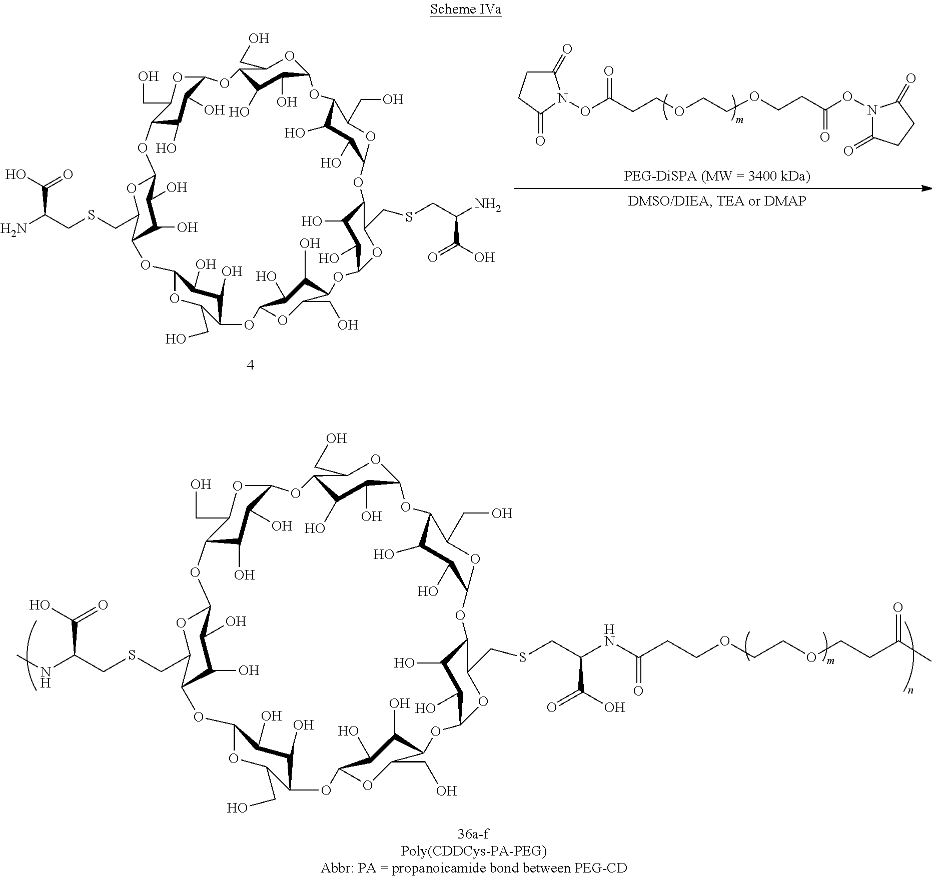

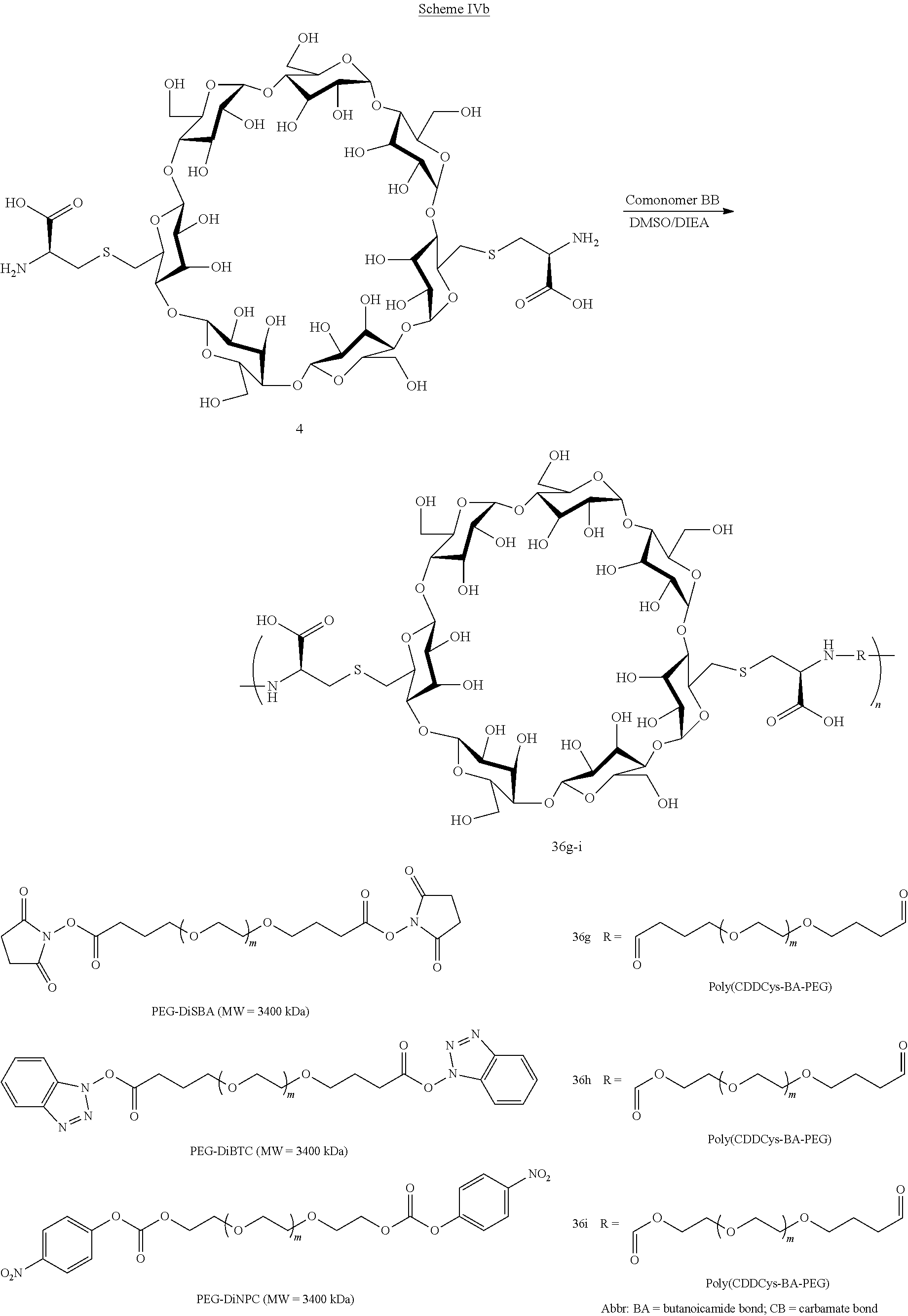

Synthesis of Poly(CDDCys-PA-PEG), 36a 4 (after precipitation with acetone, 63 mg, 0.047 mmol) and PEG-DiSPA (MW 3400, 160 mg, 0.047 mmol) were dried under vacuum for 8 hours. Anhydrous DMSO (1.26 mL) was added to the mixture under argon. After 10 minutes of stiffing, anhydrous diisopropylethylamine (DIEA, 19 μL, 2.3 eq.) was added under argon. The reaction mixture was stirred under argon for 120 h. The polymer containing solution was dialyzed using a 10,000 MWCO membrane (Spectra/Por 7) against water for 48 h and lyophilized to yield 196 mg 36a (90%, Table 1). M_{w}=57.4 kDa, Mₙ=41.7 kDa, M_{w}/Mₙ=1.38. ¹H NMR (D₂O) δ 5.08 (m, CD-2-H), 4.27 (m, Cys-CH), 2.72-3.76 (m, CD-3,4,5,6-CH, CD-CH₂, PEG-CH₂), 2.44 (m, Cys-CH₂).

Synthesis of other poly(CDDCys-PA-PEG) (36b-f), Poly(CDDCys-BA-PEG) (36g) Poly(CDDCys-CB-PEG) (36h-i) were achieved under polymerization condition similar to that of 36a. Details for the polymerization conditions, monomer selection, polymer molecular weight, polydispersity and yields are listed in Table 6. 36g: ¹H NMR (D₂O) δ 5.10 (m, CD-2-H), 4.25-4.37 (m, Cys-CH), 2.72-3.86 (m, CD-3,4,5,6-CH, CD-CH₂, PEG-CH₂), 2.21 (m, Cys-CH₂). 36h-i: ¹H NMR (D₂O) δ 5.05 (m, CD-2-H), 4.56 (m, Cys-CH), 2.70-3.93 (m, CD-3,4,5,6-CH, CD-CH₂, PEG-CH₂), 2.38 (m, —OCH₂CH₂CH₂C(O)—NH—), 2.34 (m, Cys-CH₂), 1.90 (m, —OCH₂CH₂CH₂C(O)—NH—).

Addition of a non-nucleophilic organic base (such as DIEA) was essential for this polymerization as no viscosity changes of the polymerization solutions were observed after 48 hours if no base was added. When 2.3 eq. of DIEA were added, the viscosity of the polymerization solution increased dramatically after 4-6 hours of reaction. DIEA deprotonates the amino groups of 4 to render them more nucleophilic for coupling with PEG-DiSPA. There were essentially no differences in the polymerizations if other bases, such as TEA or DMAP, were used (36b-c, Table 6). Polymerization using 4 recovered by the two different precipitation methods (acetone and methanol) produced polymers with different MWs. 4 that was purified by the methanol-precipitation method (contains no free cystine) gave higher MW polymer (36d-e) as compared to the less pure 4 that was obtained from the acetone-precipitation method (36a). Polymerization of 4 with PEG-DiSPA typically produced polymer yields greater than 90%.

4 was polymerized with other activated monomers such as PEG-DiSBA, PEG-DiBTC, and PEG-DiNPC. Reaction of 4 with PEG-DiSBA gave polymer 36g with similar linkages as 36a-f (amide bond, but one more —CH₂ group than 36a-f at the linker) with M_{w} over 100 kDa, while reaction of 4 with PEG-DiBTC and PEG-DiNPC generated polymers 36h and 36i, respectively, with connecting carbamate moiety and M_{w}'s over 50 kDa (Table 6).

TABLE 6
Polymerization of 4 with difunctionalized PEG

| | | | Polym- | | | | |
|---|---|---|---|---|---|---|---|
| | PEG | | erization | M_{w} | Mₙ | M_{w}/ | Yield |
| CDP | Comonomer | Base | time (h) | (kDa) | (kDa) | Mₙ | (%) |
| 36a^{a} | PEG-DiSPA | DIEA | 120 | 57.4 | 41.7 | 1.38 | 90 |
| 36b^{a} | PEG-DiSPA | DMAP | 120 | 54.2 | 38.1 | 1.42 | 91 |
| 36c^{a} | PEG-DiSPA | TEA | 120 | 57.4 | 42.6 | 1.35 | 91 |
| 36d^{b} | PEG-DiSPA | DIEA | 120 | 93.6 | 58.0 | 1.48 | 96 |
| 36e^{b} | PEG-DiSPA | DIEA | 144 | 97.3 | 58.0 | 1.67 | 94 |
| 36f^{b} | PEG-DiSPA | DIEA | 2 | 35.3 | 25.6 | 1.38 | 95 |
| 36g | PEG-DiSBA | DIEA | 120 | 114.7 | 77.9 | 1.47 | 96 |
| 36h | PEG-DiBTC | DIEA | 120 | 67.6 | 39.4 | 1.47 | 95 |
| 36i | PEG-DiNPC | DIEA | 120 | 86.5 | 57.2 | 1.51 | 96 |

^{a}4 was washed with acetone before polymerization.
^{b}4 was washed with methanol before polymerization.

Polymers 36a-i are highly soluble in aqueous solution. They can be easily dissolved in water or phosphate buffered saline (PBS) solution at concentrations of at least 200 mg/mL. Solubility of these polymers in aqueous solution at concentrations higher than 200 mg/mL was not attempted due to the high viscosity. These polymers were also soluble in DMF, DMSO and methanol, slightly soluble in CH₃CN and CHCl₃, but insoluble in THF and ethyl ether.

Molecular Weight Control of CD Polymers 4 (after precipitation with methanol) (56.2 mg, 0.0419 mmol) and PEG-DiSPA (147 mg, 0.0419 mmol) were dried under vacuum for 4-8 hours. To the mixture was added dry DMSO (1.1 mL) under argon. After 10 minutes stiffing, DIEA (16 μL, 2.2 eq) was added under argon. A portion of polymerization solution (150 μL) was removed and precipitated with ether at selected times (2 h, 18 h, 43 h, 70 h, 168 h and 288 h). MWs of the precipitated polymers were determined as described above.

### B. Synthesis of Poly(CDDCys-PA-PEG)-CPT Conjugates (HGGG6, LGGG10, HG6, HGGG10)

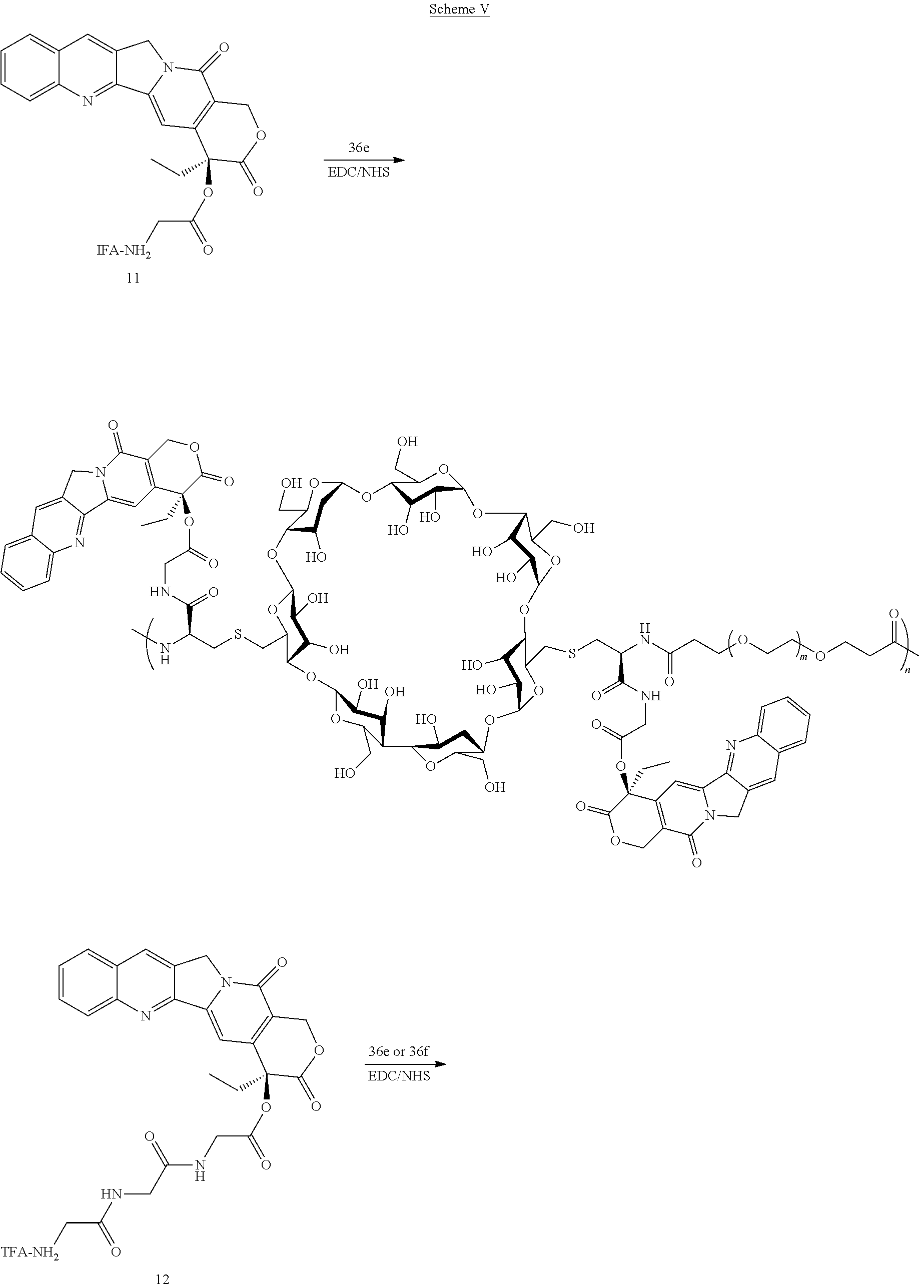

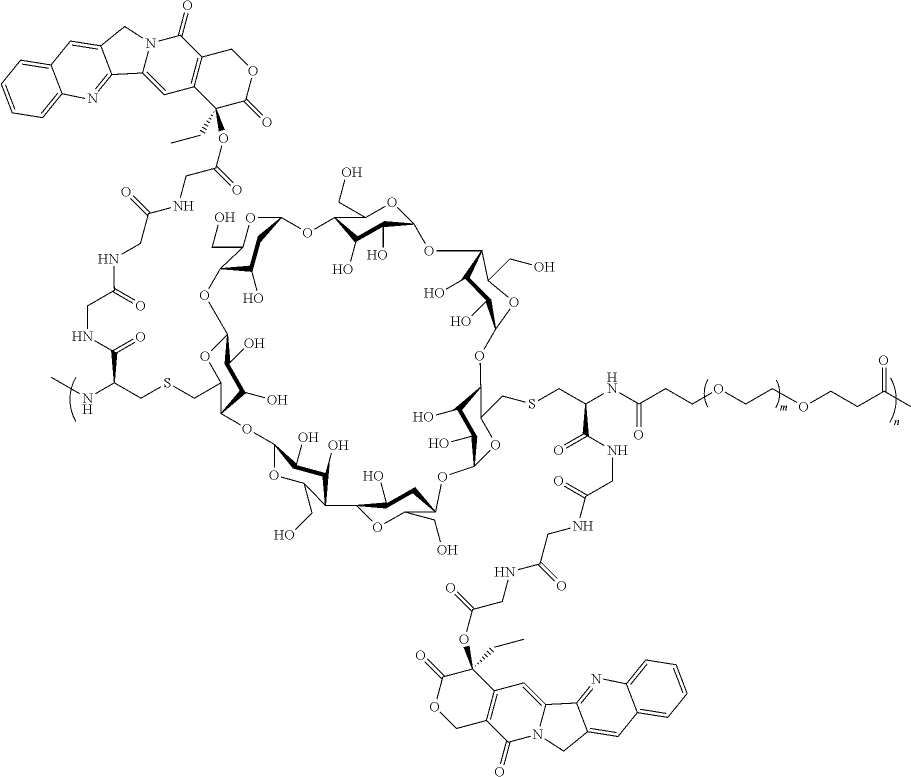

Synthesis of Poly(CDDCys-PA-PEG)-GlyGlyGly-CPT (HGGG6) 36e (1.37 g, 0.30 mmol of repeat unit) was dissolved in dry DMSO (136 mL). The mixture was stirred for 10 minutes. 12 (419 mg, 0.712 mmol, 2.36 eq), DIEA (0.092 mL, 0.712 mmol, 2.36 eq), EDC (172 mg, 0.903 mmol, 3 eq), and NHS (76 mg, 0.662 mmol, 2.2 eq) were added to the polymer solution and stirred for ca. 15 hours. The polymer was precipitated with ethyl ether (1 L). The ether was poured out and the precipitate was washed with CH₃CN (3×100 mL). The precipitate was dissolved in water 600 mL. Some insoluble solid was filtered through 0.2 μm filters. The solution was dialyzed using 25,000 MWCO membrane (Spectra/Por 7) for 10 h at 10-15° C. in DI water. Dialysis water was changed every 60 minutes. The polymer-drug conjugate solution was sterilized by passing it through 0.2 μM filters. The solution was lyophilized to yield a yellow solid HGGG6 (1.42 g, 85% yield).

Synthesis of Poly(CDDCys-PA-PEG)-GlyGlyGly-CPT (LGGG10) Conjugation of 12 to 36f was performed in a manner similar to that used to produce HGGG6 except that this conjugate was dialyzed with 10,000 MWCO membrane (Spectra/Por 7) instead of with 25,000 MWCO membrane. The yield of LGGG10 was 83%.

Synthesis of Poly(CDDCys-PA-PEG)-Gly-CPT (HG6) Conjugation of 11 to 36e was performed in a manner similar to that used to produce HGGG6. The yield of HG6 was 83%.

Synthesis of Poly(CDDCys-PA-PEG)-GlyGlyGly-CPT (HGGG10) 36e (1.5 g, 0.33 mmol of repeat unit) was dissolved in dry DMSO (150 mL). The mixture was stirred for 10 minutes. 12 (941 mg, 1.49 mmol, 4.5 eq), DIEA (0.258 mL, 1.49 mmol, 4.5 eq), EDC (283 mg, 1.49 mmol, 4.5 eq), and NHS (113 mg, 0.99 mmol, 3 eq) was added to the polymer solution and stirred for ca. 24 hours. Another portion of EDC (142 mg, 0.75 mmol, 2.3 eq) and NHS (56 mg, 0.5 mmol, 1.5 eq) were added to the conjugation solution. The polymer was stirred for an additional 22 hours. The workup procedure was the same as that for the synthesis of HGGG6. The yield of HGGG10 was 77%.

### Determination of wt % CPT on the Conjugates

Stock solutions of HGGG6, LGGG10, HG6 and HGGG10 were prepared at a concentration of 10 mg/mL in DMSO. An aliquot of corresponding stock solution was diluted to 100 μg/mL using 1 N NaOH. CPT was completely hydrolyzed in this basic solution and transformed to its carboxylate form within 2 h at room temperature. An aliquot of this solution was diluted to 10 μg/mL using 8.5% H₃PO₄, and the CPT carboxylate form was transformed to its lactone form. 30 μL of this solution was injected into the HPLC. The peak area from the CPT lactone form was integrated and compared to a standard curve.

11 and 12 were conjugated to 36e or 36f (Table 2) using conventional coupling methods. Due to the instability of the ester linker of 11 and 12 in aqueous solution, the conjugation was conducted in anhydrous DMSO under argon. An organic base was required to deprotonate the TFA salts of 11 and 12 to facilitate the coupling. For polymer conjugation with 12, the weight percent (wt %) drug loading was around 6-10%. The theoretical maximum drug loading is around 13% using PEG with MW of 3400 Da; maximum values can be increased by decreasing the MW of the PEG segments. Solubilities of all conjugates in water or PBS were more than 200 mg/mL (equivalent to a 12-20 mg CPT/mL for 6-10 wt % drug loading, respectively). Details for the HGGG6, LGGG10, HG6, and HGGG10 are summarized in Table 7.

TABLE 7
Properties of polymer-CPT conjugates.

| | M_{w} of parent polymer | | | |
|---|---|---|---|---|
| Conjugate^{a} | (×10⁻³) | M_{w}/Mₙ^{b} | Linker | CPT (wt %) |
| HGGG6 | 97 | 1.7 | triglycine | 6.1 |
| LGGG10 | 35 | 1.6 | triglycine | 10.2 |
| HG6 | 97 | 1.7 | glycine | 6.8 |
| HGGG10 | 97 | 1.7 | triglycine | 9.6 |

^{a}Abbreviations: H = High M_{w} polymer (97 kDa), L = Low M_{w} polymer (35 kDa), GGG = triglycine linker, G = glycine linker, 6 = drug loading around 6 wt %, 10 = drug loading around 10 wt %.
^{b}Polymer polydispersity as measured by light scattering techniques(26)

### C. Release of CPT from HGGG6 and HG6

### Release of CPT in PBS

HGGG6 and HG6 were prepared at 1 mg/mL in PBS (1×, pH 7.4). A 100 μL aliquot of the solution was transferred to a 1.5 mL Eppendorf tube and incubated at 37° C. The incubated samples were quenched at selected time intervals and stored at −80° C. until the analysis. Each solution was diluted with 8.5% H₃PO₄ to a 5 mL total volume in a volumetric flask. 30 μL of such solution was injected into the HPLC. The peak area from the CPT lactone form was integrated and compared to a standard curve.

Analysis for the release of CPT from HGGG6 and HG6 in PBS containing acetyl cholinesterase (an esterase, 100 units/mL), in KH₂PO₄ buffer (pH 6.1, 0.1 M) and in the KH₂PO₄ buffer (pH 6.1, 0.1 M) containing cathepsin B (a cysteine proteinase, 200 μM, preactivated on ice for 30 minutes in this buffer containing 2 mM DTT and 1 mM EDTA) were performed in a manner similar to that described above for PBS alone.

### Release of CPT in Human Plasma

An aliquot of HGGG6 and HG6 stock solution were diluted to give final concentration of 0.5 mg/mL in PBS (1×, pH 7.4). This solution was added to a lyophilized powder of human plasma to reconstitute 100% human plasma by the recommended amount. The solution was divided into equal volume (250 μL) to 1.5 mL Eppendorf tubes, incubated at 37° C., and stopped at selected time point. Samples were stored at −80° C. until the analysis. Samples were separated from plasma by solid phase extraction columns The solid phase extraction cartridge (Oasis HLB 1 cc cartridge from Waters) was pre-conditioned with 1 mL of acetonitrile and then with 1 mL of 8.5% H₃PO₄ before loading. Samples were acidified with equal volume of 8.5% H₃PO₄ prior to loading. After the acidified solution was loaded on the cartridge, the bed was washed with 3×1 mL of water. Released CPT and polymer conjugate were eluted with 3×1 mL of a solution mixture of acetonitrile and potassium phosphate buffer (pH 4.1) (60/40 v/v). The eluted solution was diluted to 5 mL total volume in a 5 mL volumetric flask. 30 μL of such solution was injected into the HPLC. The peak area from the CPT lactone form was integrated and compared to a standard curve.

Release of CPT from HGGG6 and HG6 in PBS containing 4% human plasma (PBS/reconstituted human plasma solution=96/4 (v/v)), in mouse plasma and in reconstituted human albumin (PBS solution) were performed in a manner similar to that described above for pure human plasma.

In PBS (1×, pH 7.4), the half-lives (t_{1/2}) for releasing CPT from HG6 and HGGG6 were 59 h and 32 h, respectively. The half-lives decreased to 25 h and 22 h, respectively, in the presence of 4% human plasma, and to 1.7 h and 1.6 h, respectively, in 100% human plasma (“HP”) and 2.6 h and 2.2 h, respectively, in 100% mouse plasma (“MP”). CPT release rates for both HG6 and HGGG6 in the presence of albumin (“Alb”) or acetyl cholinesterase (“Ac Cho”) were on the same order of magnitude as in PBS. In a buffer solution at a pH lower than PBS (pH 6.1) with or without the enzyme cathepsin B (active at pH 6.1), less than 50% of total conjugated CPT was released from both HG6 and HGGG6 for times up to 144 h (Table 8).

TABLE 8
Half-life (t_{1/2}, in hour) of the release of CPT from HG6 and HGGG6^{a}

| | | | | | | | | Cath B |
|---|---|---|---|---|---|---|---|---|
| | | 4% | | | | Ac | pH 6.1 | (pH |
| Conjugate | PBS^{b} | HP^{c} | HP^{d} | MP^{e} | Alb^{f} | Cho^{g} | buffer^{h} | 6.1)ⁱ |
| HG6 | 59 | 25 | 1.7 | 2.6 | 62 | 33 | >144 | >144 |
| HGGG6 | 32 | 22 | 1.6 | 2.2 | 73 | 43 | >144 | >144 |

^{a}t_{1/2} is defined as time (hours) for the release of half of the total conjugated CPT.
Abbreviations:
HP means human plasma,
MP means mouse plasma.
^{b}pH 7.4 PBS 1x buffer.
^{c}Reconstituted human plasma mixed with PBS (v/v = 4/96).
^{d}Reconstituted human plasma
^{e}Fresh mouse plasma
^{f}In reconstituted human albumin PBS buffer
^{g}In the presence of acetyl cholinesterase PBS solution (100 units/mL).
^{h}pH 6.1 phosphate buffer (0.1M)
ⁱpH 6.1 phosphate buffer in the presence of Cathepsin B

### Release of CPT in Solution at Different pH.

HGGG6 and HG6 were prepared at 1 mg/mL in buffer solution with pHs ranging from acidic (pH=1.2) to basic (pH=13.1) and incubated at 37° C. for 24 h. An aliquot of each solution was diluted with 8.5% H₃PO₄ to about 100 μg/mL. 30 μL of such solution was injected into HPLC. The peak area from the CPT lactone form was integrated and compared to a standard curve.

The pH of aqueous solution has a significant effect on the CPT release rates from both HG6 and HGGG6. The glycinyl-CPT ester bonds of both HG6 and HGGG6 were very stable in acidic pH (1.1 to 6.4) as less than 7% of CPT were released in 24 h.

Methods for Increasing Drug Weight Percent Loading

Method I. Synthesis of CD-BisCys-Peg Copolymer with a Short Peg Linkage and its GlyCPT Conjugate

### Example 5

### Synthesis of CD-BisCys-Peg (Short PEG, e.g., Peg200-Peg2000) and its CPT Conjugate 42

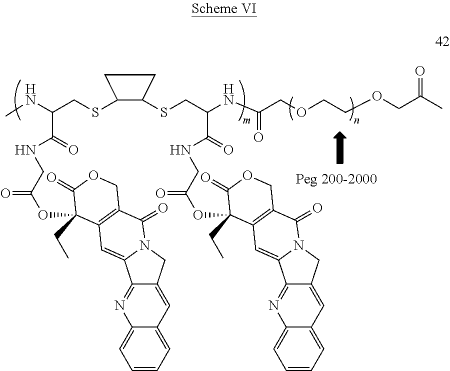

Synthesis of polymer and drug conjugate 42 are same as 36, 37, and 38

While Scheme VI shows that the drug is attached at all available positions, not all positions may be reacted. Therefore, a particle comprising conjugates described above may include a conjugate reacted at all positions available for attachment and particles that have less than all of the positions available for attachment containing the drug, e.g., the particle can include CPD reacted at one or none of the positions available for attachment. Thus, while Scheme VI depicts CPT at every point of attachment of each polymer subunit, the CDP-CPT conjugate can have less than 2 CPT molecules attached to any given polymer subunit of the CDP. For example, in one embodiment, the CDP-CPT conjugate includes several polymer subunits and each of the polymer subunits can independently include two, one or no CPT attached at each point of attachment of the polymer subunit. In addition, the particles and compositions can include CDP-CPT conjugates having two, one or no CPT attached to each polymer subunit of the CDP-CPT conjugate and the conjugates can also include a mixture of CDP-CPT conjugates that can vary as to the number of CPTs attached at each point of attachment of the polymer subunits of the conjugates in the particle or composition.

Method II. Synthesis of CD-BisCys-Peg Copolymer with Multiple Drug Molecules on Each Loading Site.

### Example 6

### Synthesis of CD-BisCys-Peg and its GluBis(GlyCPT) Conjugate 43

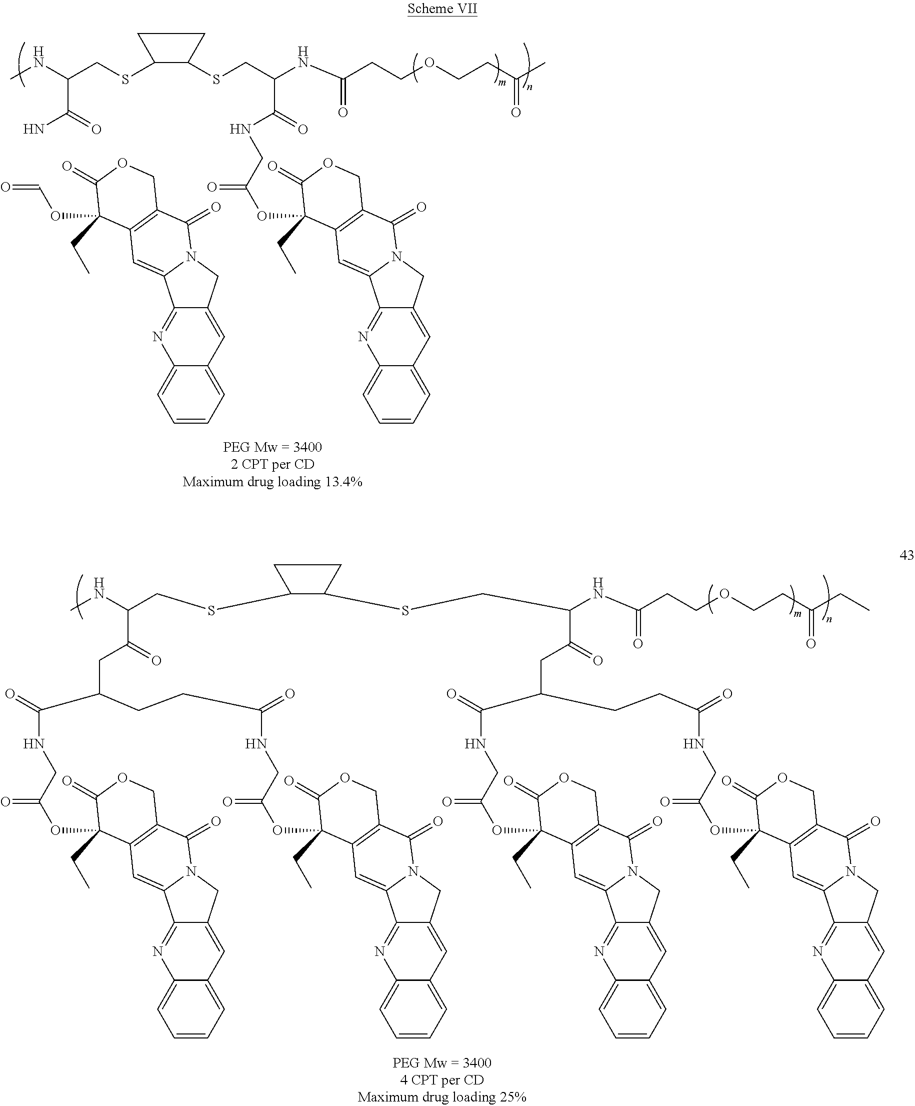

36 and Glu-Bis(Gly-CPT) 17 are dissolved in DMSO. EDC (3 eq), NHS (2.2 eq), and DIEA (2.2 eq) are added to the solution. CD-BisCys-Peg-GluBis(GlyCPT) 43 is precipitated with CH₃CN and washed with the same solvent until no free drug is detected using UV or TLC. 43 is dried under high vacuum. While Scheme VII shows that the drug is attached at all available positions, not all positions may be reacted. Therefore, a particle comprising conjugates described above may include a conjugate reacted at all positions available for attachment and particles that have less than all of the positions available for attachment containing the drug, e.g., the particle can include CDP reacted at three, two, one or none of the positions available for attachment. Thus, while Scheme VII depicts CPT at every point of attachment of each polymer subunit, the CDP-CPT conjugate can have less than 4 CPT molecules attached to any given polymer subunit of the CDP. For example, in one embodiment, the CDP-CPT conjugate includes several polymer subunits and each of the polymer subunits can independently include four, three, two, one or no CPT attached at each point of attachment of the polymer subunit. In addition, the particles and compositions can include CDP-CPT conjugates having four, three, two, one or no CPT attached to each polymer subunit of the CDP-CPT conjugate and the conjugates can also include a mixture of CDP-CPT conjugates that can vary as to the number of CPTs attached at each point of attachment of the polymer subunits of the conjugates in the particle or composition.

### Example 7

### Synthesis and In Vitro Analysis of CDP-Gly-SN-38

SN-38 was derivatized with the amino acid glycine at the 20-OH position as shown in Scheme VIII. Briefly, 20(S)-7-ethyl-10-hydroxycamptothecin (SN-38, 1.0 g, mmol) was dissolved in a mixture of 70 mL dimethylformamide (DMF) and 30 mL pyridine. A solution of di-tert-butyl-dicarbonate (0.83 g, 3.8 mmol) in 10 mL DMF was added and the mixture stirred at room temperature overnight (12 hours). The solvent was removed under vacuum to yield a yellow solid and re-crystallized from boiling 2-propanol (75 mL) to yield 20(s)-10-tert-butoxycarbonyloxy-7-ethylcamptothecin (Boc-SN-38) as a yellow solid (0.6 g, 48% yield).

Boc-SN-38 (0.73 g, 1.5 mmol), N-(tertbutoxycarbonyl)glycine (0.26 g, 1.5 mmol) and 4-dimethylaminopyridine (DMAP, 0.18 g, 1.5 mmol) were dissolved in anhydrous methylene chloride (30 mL) and chilled to 0° C. 1,3-Diisopropyl-carbodiimide (DIPC, 0.19 g, 1.5 mmol) was added, the mixture stirred at 0° C. for 30 minutes followed by stiffing for 4 hours at room temperature. The mixture was diluted with methylene chloride to 100 mL, washed twice with an aqueous solution of 0.1N hydrochloric acid (25 mL), dried over magnesium sulfate and the solvent removed under vacuum. The resulting yellow solid was purified by flash chromatography in methylene chloride:acetone (9:1) followed by solvent removal under vacuum to yield 20-O—(N-(tert-butoxycarbonyl)glycyl)-10-tert-butyoxycarbonyloxy-7-ethylcamptothecin (diBoc-Gly-SN-38, 640 mg, 67% yield).

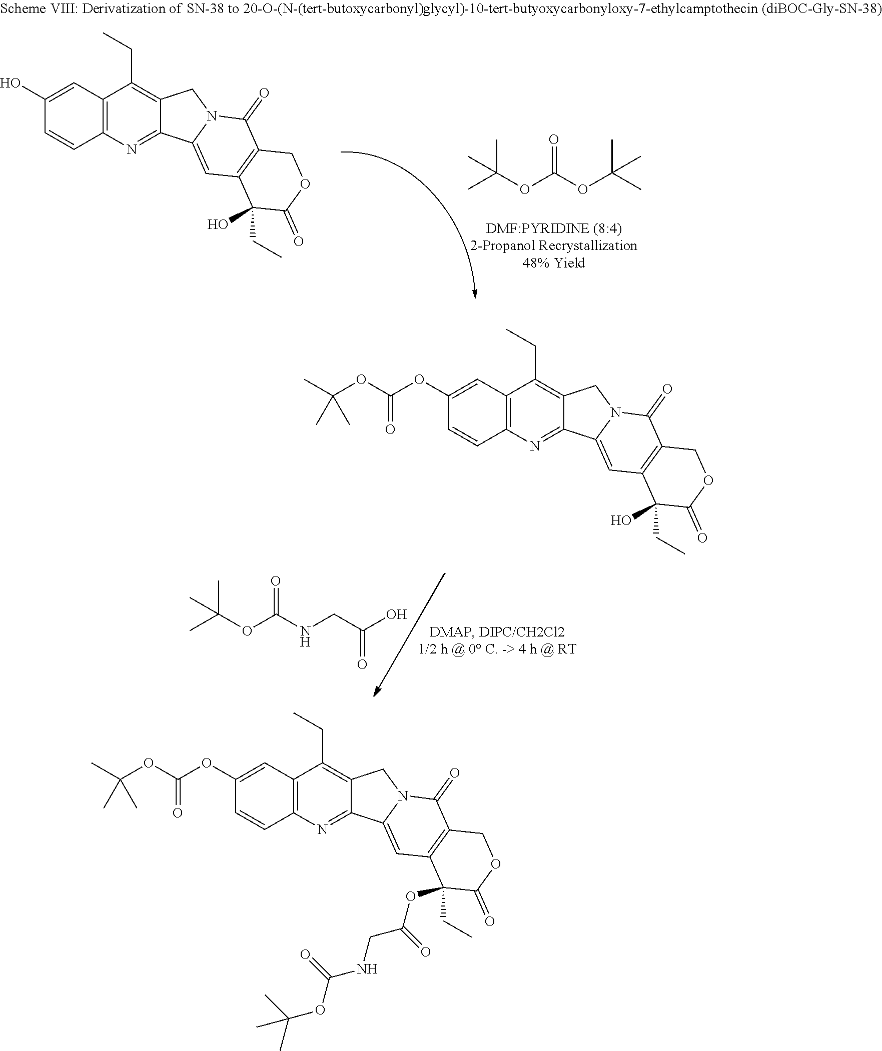 [SMILES: CCc1c2c(nc3ccc(O)cc13)-c1cc3c(c(=O)n1C2)COC(=O)[C@]3(O)CC ; CC(C)(C)OC(=O)OC(C)(C)C ; CCc1c2c(nc3ccc(OC(=O)OC(C)(C)C)cc13)-c1cc3c(c(=O)n1C2)COC(=O)[C@]3(O)CC ; CC(C)(C)OC(=O)NCC(=O)O ; CCc1c2c(nc3ccc(OC(=O)OC(C)(C)C)cc13)-c1cc3c(c(=O)n1C2)COC(=O)[C@@]3(CC)OC(=O)CNC(=O)OC(C)(C)C]

CDP was synthesized as previously described (Cheng et al. (2003) Bioconjugate Chemistry 14(5):1007-1017). diBOC-Gly-SN-38 (0.62 g, 0.77 mmol) was deprotected in 15 mL of a 1:1 mixture of methylene chloride:trifluoroacetic acid (TFA) at room temperature for 1 hour. 20-O-trifluoroglycine-10-hydroxy-7-ethylcamptothecin (TFA-Gly-SN-38, 0.57 g, 97% yield) was isolated as a yellow solid by precipitation with ethanol (100 mL), followed by two washes with ethanol (30 mL each), dissolution in methylene chloride and removal of solvent under vacuum. ESI/MS expected 449.4. Found 471.66 (M+Na).

CDP-Gly-SN-38 (Poly-CD-PEG-Gly-SN-38, scheme IX) was synthesized as follows: CDP (270 mg, 0.056 mmol), TFA-Gly-SN-38 (70 mg, 0.12 mmol), N-hydroxy-succinimide (14 mg, 0.12 mmol), and 1-ethyl-3-(3-dimethylaminopropyl) carbodiimide (EDCI, 32 mg, 0.17 mmol) were dissolved in dimethylformamide (10 mL) and stirred for 4 hours at room temperature. The polymer was precipitated by addition of 50 mL acetone followed by 50 mL diethyl ether. Precipitate was centrifuged, washed twice with 20 mL acetone each, and dissolved in water acidified to pH 3.0 with hydrochloric acid. Polymer solution was dialized for 24 hours against pH 3.0 water using a 25 kDa MWCO dialysis membrane. The resulting solution was lyophilized to yield CDP-Gly-SN-38 (180 mg, 67% yield). The polymer was analyzed for total and free SN-38 content by HPLC using SN-38 as a standard curve as previously described (Cheng et al. (2003) Bioconjugate Chemistry 14(5):1007-1017). Total SN-38 content was 7.66% w/w of which 97.4% was polymer bound. Average particle size was determined by dynamic light scattering to be 27.9 nm.

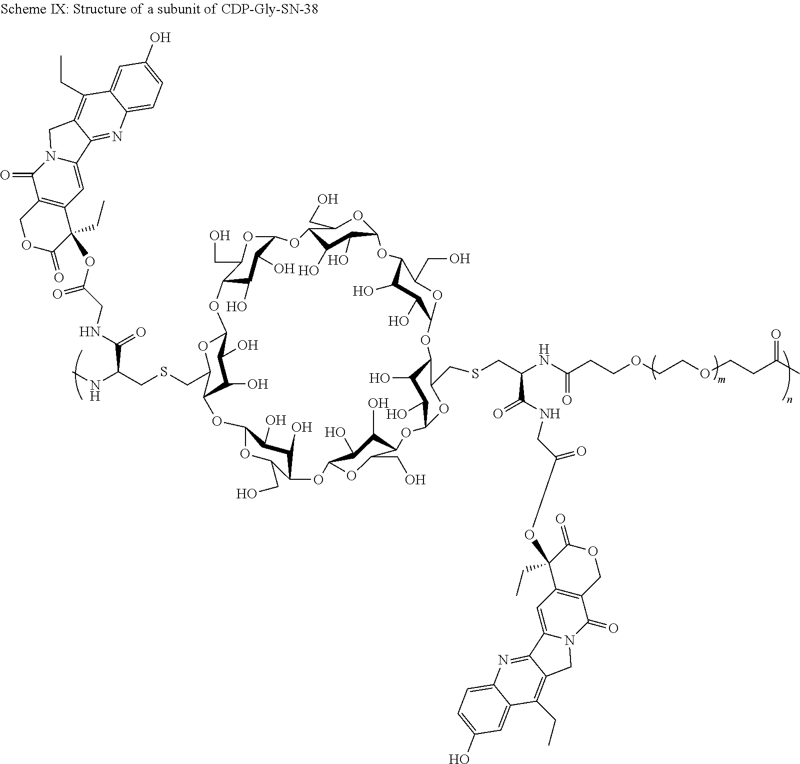

While Scheme IX shows that the drug is attached at all available positions in the subunit, not all positions may be reacted. Therefore, a particle comprising conjugates described above may include a conjugate reacted at all positions available for attachment and particles that have less than all of the positions available for attachment containing the drug, e.g., the particle can include CPD reacted at one or none of the positions available for attachment. Thus, while Scheme IX depicts SN-38 at every point of attachment of each polymer subunit, the CDP-SN-38 conjugate can have less than 2 SN-38 molecules attached to any given polymer subunit of the CDP. For example, in one embodiment, the CDP-SN-38 conjugate includes several polymer subunits and each of the polymer subunits can independently include two, one or no Sn-38 attached at each point of attachment of the polymer subunit. In addition, the particles and compositions can include CDP-SN-38 conjugates having two, one or no Sn-38 attached to each polymer subunit of the CDP-Sn-38 conjugate and the conjugates can also include a mixture of CDP-Sn-38 conjugates that can vary as to the number of Sn-38s attached at each point of attachment of the polymer subunits of the conjugates in the particle or composition.

### In Vitro Evaluation of CDP-Gly-SN-38

CDP-Gly-SN-38 was evaluated in A2780 human ovarian cancer cell lines in vitro as follows:

The human ovarian carcinoma A2780 cells were obtained from the American Type Culture Collection. Cells were seeded in 96-well plates at a concentration of 5,000 cells per well and grown in medium containing 10% fetal bovine serum at 37° C. for 24 h in a humidified 5% CO₂ atmosphere. The medium was replaced with fresh medium containing the test compound at concentrations ranging from 0.01 nmol/L to 1 μmol/L. Triplicate wells per plate were treated at each concentration. Controls were vehicle-treated cells and medium only blank. Plates were incubated at 37° C. for 72 h. MTS assay reagent was prepared by diluting CellTiter 96 AQueous One Solution (Promega) 5-fold into PBS/glucose (4.5 g/L). Cell culture medium was aspirated and 100 μL of MTS reagent were added to each well. Plates were incubated at 37° C. for 1 h. The plates were shaken for 5 min and the absorbance was measured at 485 nm using a SPECTRAFluor Plus plate reader (Tecan). The percentage of cell survival was calculated relative to untreated cells, and IC50s were estimated from the graphs of log dose (nmol/L) versus % cell survival (GraphPad Prizm).

The results of this experiment are shown in Table 9 below.

TABLE 9
IC50 of SN-38 and CDP-Gly-SN-38 on A2780 ovarian cells.

| Compound | IC50 (nM) |
|---|---|
| SN-38 | 2.44 |
| CDP-PEG-Gly-SN-38 | 7.22 |

### Example 8

### CRLX101 Causes a Dose-Dependent Inhibition of Tumor Growth in a Non-Small Cell Lung Cancer Model (A549) Possessing a KRAS Mutation

A549 cells were obtained from the American Type Culture Collection (ATCC). A549 cells (passage=4) were grown in culture to 85-90% confluency in F-12K medium supplemented 10% FBS and 1% penicillin/streptomycin and then resuspended in F-12K medium supplemented with 30% Matrigel. A549 cells (5×10⁶ cells in 100 μL) were implanted subcutaneously into the mammary fat pad of 9 week old male Taconic NCR nu/nu mice (23.3-32.5 g) on Day 1.

Animals were randomly divided into four groups and treatment was started on day-16 post implantation, when the mean tumor volume was between 170 and 180 mm³. The four treatment groups that were administered to the mice were: (1) PBS (vehicle control group), i.v. once weekly for three weeks (qwk×3); (2) CRLX101 i.v. at 2 mg/kg (active compound camptothecin equivalent) qwk×3; (3) CRLX101 i.v. at 4 mg/kg (active compound camptothecin equivalent) qwk×3; (4) CRLX101 i.v. at 6 mg/kg (active compound camptothecin equivalent) qwk×3.

Animals were monitored for any morbidity and adverse effect three times a week. Body weight and tumor volume were also measured three times a week. Each animal was euthanized when the calculated tumor volume reached the 1000 mm³ endpoint. Tumor volume was calculated with the following equation: (width×width×length)/2 mm³. Efficacy was determined by tumor growth inhibition (TGI), tumor growth delay (TGD) and survival. Tumor growth delay (TGD) was calculated by the difference between the day when the treatment group average tumor size reached the maximum tumor volume of 1000 mm³ and the day when the vehicle-treated group reached an average tumor volume of 1000 mm³. TGI was represented as % and calculated as follows: (1-(treated tumor volume/control tumor volume))×100 when the control group mean tumor volume reached ≧1000 mm³. Tolerability was determined by changes in body weight, expressed as a percent of the initial body weight on post-implantation day-16. The criteria at which a mouse was removed from the study were >20% body weight loss or severe morbidity or hind limb paralysis.

| | | | | Tumor | Tumor | Maximum |
|---|---|---|---|---|---|---|
| | | | Dose | growth | growth | body |
| Group | Treatment | | (mg/ | inhibition | delay | weight |
| # | Regimen | Schedule | kg) | (%) | (days) | loss (%) |
| 1 | Vehicle | q7d x 3* | N/A | N/A | 21.8 | 0 |
| 2 | CRLX101 | q7d x 3 | 2 | 23.1 | 27.0 | 0 |
| 3 | CRLX101 | q7d x 3 | 4 | 63.0 | >48** | 0 |
| 4 | CRLX101 | q7d x 3 | 6 | 73.9 | >48** | 3.6 |

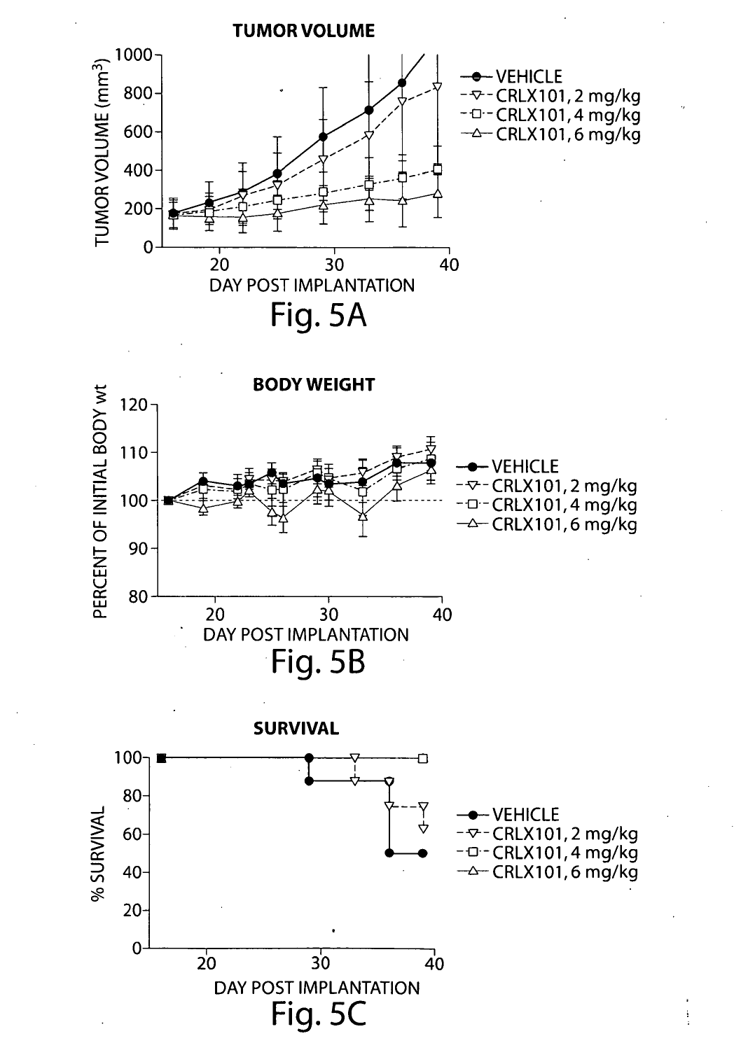

*q7d x 3 = three injections, seven days apart.
**Accurate TGD not yet available - study not yet completed
CRLX101 at 4 mg/kg and 6 mg/kg qwk x 3 resulted in a decrease in tumor volume (FIG. 5A) and survival time (FIG. 5C) as compared to PBS alone (control) in the A549 mice having the KRAS mutation.

### Example 9

### CRLX101 Causes a Dose-Dependent Inhibition of Tumor Growth in a Non-Small Cell Lung Cancer Model (NCl-H2122) Possessing a KRAS Mutation

NCI-H2122 cells (passage=4) were grown in culture to 85-90% confluency in RPMI-1640 medium supplemented 10% FBS and 1% penicillin/streptomycin and then resuspended in RPMI-1640 medium. H2122 cells (5×10⁶ cells in 100 μL) were implanted subcutaneously into the mammary fat pad area of 9 week old female Harlan nu/nu mice (21.6-25.6 g) on Day 1.

Animals were randomly divided into four groups and treatment was started on day-14 post implantation, when the mean tumor volume was between 180 and 185 mm³. The four treatment groups that were administered to the mice were: (1) PBS (vehicle control group), i.v. once weekly for three weeks (qwk×3); (2) CRLX101 i.v. at 2 mg/kg (active compound camptothecin equivalent) qwk×3; (3) CRLX101 i.v. at 4 mg/kg (active compound camptothecin equivalent) qwk×3; (4) CRLX101 i.v. at 6 mg/kg (active compound camptothecin equivalent) qwk×3.

Animals were monitored for any morbidity and adverse effect three times a week. Body weight and tumor volume were also measured three times a week. Each animal was euthanized when the calculated tumor volume reached the 1000 mm³ endpoint. Tumor volume was calculated with the following equation: (width×width×length)/2 mm³. Efficacy was determined by tumor growth inhibition (TGI), tumor growth delay (TGD) and survival. Tumor growth delay (TGD) was calculated by the difference between the day when the treatment group average tumor size reached the maximum tumor volume of 1000 mm³ and the day when the vehicle-treated group reached an average tumor volume of 1000 mm³. TGI was represented as % and calculated as follows: (1−(treated tumor volume/control tumor volume))×100 when the control group mean tumor volume reached ≧1000 mm³. Tolerability was determined by changes in body weight, expressed as a percent of the initial body weight on post-implantation day-16. The criteria at which a mouse was removed from the study were >20% body weight loss or severe morbidity or hind limb paralysis.

| | | | | Tumor | Tumor | Maximum |
|---|---|---|---|---|---|---|
| | | | Dose | growth | growth | body |
| Group | Treatment | | (mg/ | inhibition | delay | weight |
| # | Regimen | Schedule | kg) | (%) | (days) | loss (%) |
| 1 | Vehicle | q7d x 3* | N/A | N/A | 12.6 | 5 |
| 2 | CRLX101 | q7d x 3 | 2 | 72.6 | 43.0 | 10 |
| 3 | CRLX101 | q7d x 3 | 4 | 80.2 | >46** | 14 |
| 4 | CRLX101 | q7d x 3 | 6 | 81.5 | >46** | 14 |

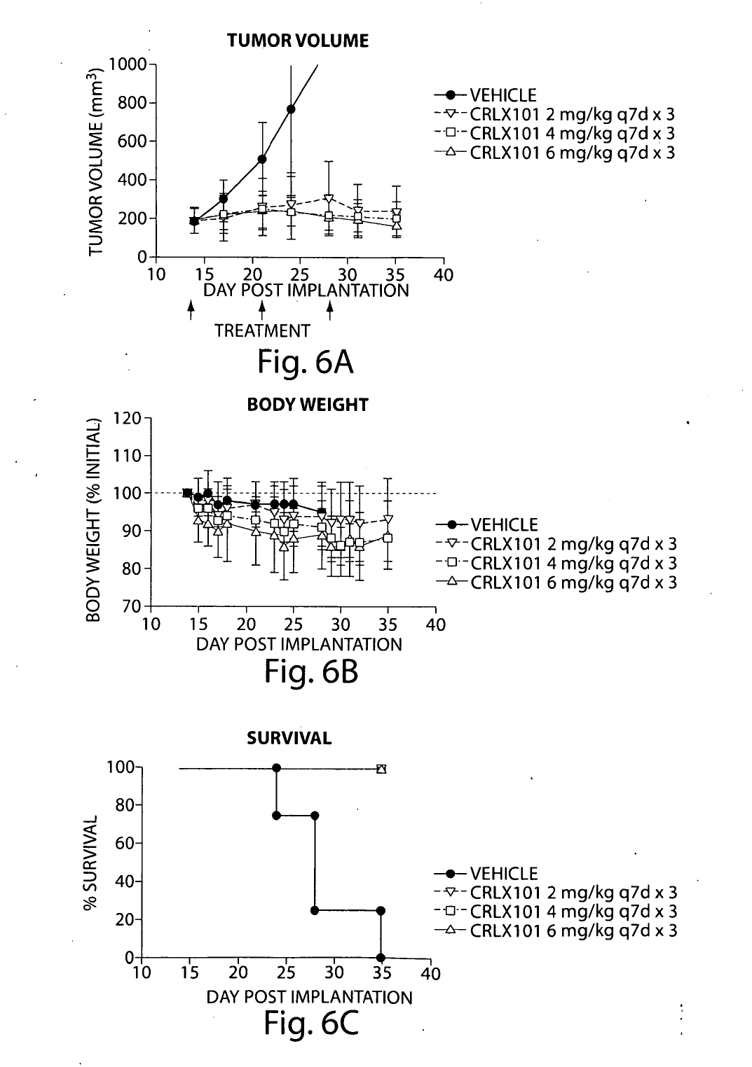

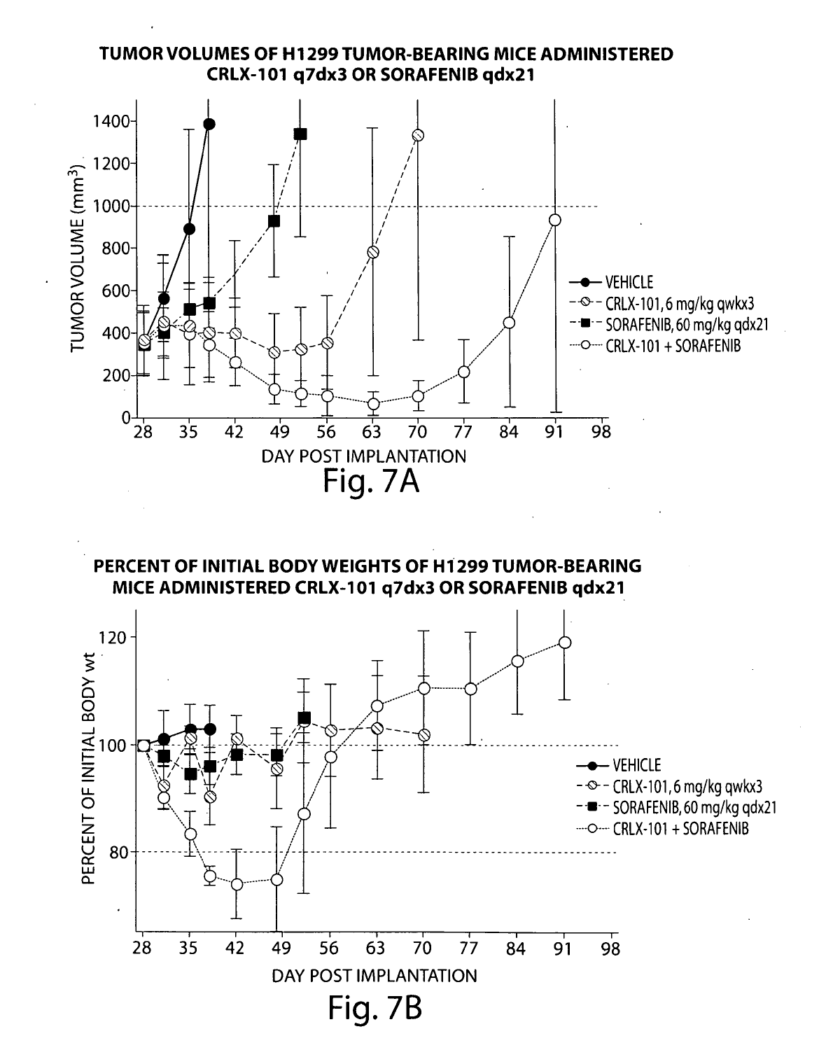

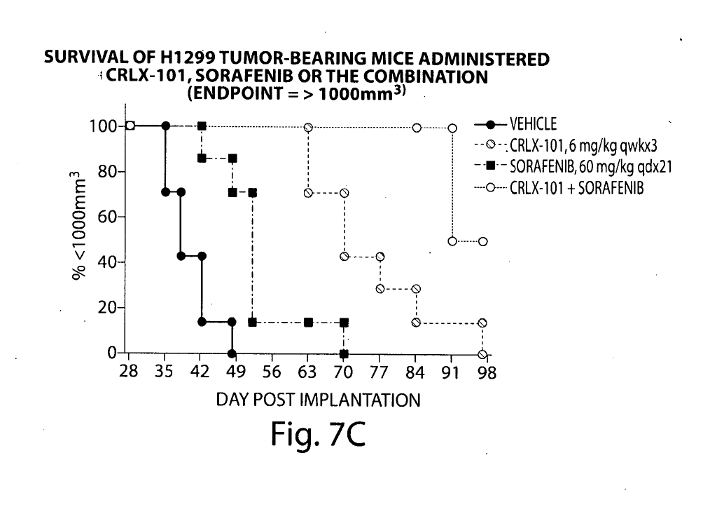

*q7d x 3 = three injections, seven days apart.
**Accurate TGD not yet available - study not yet completed
CRLX101 at 4 mg/kg and 6 mg/kg qwk x 3 resulted in a decrease in tumor volume (FIG. 6A) and survival time (FIG. 6C) as compared to PBS alone (control) in the A549 mice having the KRAS mutation.

### Example 10

### CRLX101 is Superior to Most Approved First and Second Line Treatments of Non Small Cell Lung Cancer (NSCLC) and Exhibits 100% Tumor Free Survival in H1299 Xenograft Model

H1299 cells were obtained from the American Type Culture Collection (ATCC), and the tumor line was maintained by serial engraftment in nude mice. A tumor fragment (˜1 mm³) was implanted subcutaneously in the right flank of test animals (9-week old female nu/nu mice, Harlan, body weight 18.8-25.8 gm) on day-1 of the study Animals were randomly divided into seven groups and treatment was started on day-15 post implantation, when the mean tumor volume was approximately 115 mm³

The seven treatment groups that were administered to the mice were: (1) PBS (vehicle control group), i.v. once weekly for three weeks (qwk×3), (2) gemcitabine i.p. at 120 mg/kg once every three days for four doses (q3d×4); (3) docetaxel i.v. at 30 mg/kg qwk×3; (4) topotecan i.p. at 12 mg/kg once every four days for three doses (q4d×3); (5) erlotinib orally (p.o.) at 100 mg/kg once daily for 21 days (qd×21); (6) pemetrexed p.o. at 500 mg/kg qd×14; (7) CRLX101 i.v. at 6 mg/kg (active compound camptothecin equivalent) qwk×3; and (8) CRLX101 i.v. at 6 mg/kg (active compound camptothecin equivalent) qwk×3.

Body weight was measured daily on Days 1-5, then twice weekly until the completion of the study. Tumor volume was measured twice weekly and each animal was euthanized when the calculated tumor volume reached the 2000 mm³ endpoint, or on day-88 post implantation, whichever came first. Treatment evaluations were based on tumor growth delay (TGD), defined as the increase in the median time-to-endpoint (TTE) in drug-treated versus vehicle-treated mice, on the logrank significance of survival extensions on day-88 (74 days post first treatment) survival rates, and on regression responses. Tumors in all control mice reached the volume endpoint with a median TTE of 24.4 days, allowing a maximum possible TGD of 49.6 days (203%) in the study.

Gemcitabine, docetaxel, and topotecan therapies each provided significant survival extension (P<0.001). Gemcitabine at 120 mg/kg resulted in a median TTE of 68.7 days, corresponding to 44.3-day TGD (182%), five tumor-free survivors (TFS) with a median tumor volume (MTV) of 0 mm3, two partial regressions (PRs), and one transient complete regression (CR). Docetaxel at 30 mg/kg resulted in 13.4-day TGD (55%), two D74 survivors with an MTV of 302 mm3, and one PR. Topotecan at 12 mg/kg resulted in 14.8-day TGD (61%), and one TFS. Docetaxel therapy resulted in acceptable 9.3% group mean body weight loss on D21; mean weight losses were <3% with all other drugs. Erlotinib and pemetrexed produced non-significant survival extensions. Erlotinib at 100 mg/kg resulted in 4.7-day TGD (19%), pemetrexed at 500 mg/kg resulted in 1.9-day TGD (8%), and neither drug yielded D74 survivors or regressions. Both were well-tolerated. Two deaths among pemetrexed-treated mice were classified as non-treatment-related. CRLX101 at 10 mg/kg was well tolerated and resulted in assigned median TTEs of 74 days post first treatment, corresponding to the maximum possible 49.6-day TGD (203%), and yielded 100% complete response and survivors.

In summary, CRLX101 at 10 mg/kg qwk×3 resulted in 100% tumor-free survival for 74 days (post first treatment) in the H1299 human NSCLC xenograft model. In comparison, among the marketed drugs administered on optimal preclinical regimens: gemcitabine yielded 50% TFS and three transient regressions; topotecan yielded 10% TFS; docetaxel yielded 20% survival and no TFS; and erlotinib and pemetrexed each had non-significant activities. Docetaxel caused body weight loss within the acceptable limit (<20%) and CRLX101 and all other treatment agents were well-tolerated, except that one animal dosed at 10 mg/kg experienced >15% BW loss on Days 18-25, and was documented to be thin, hunched, and have an impaired gait on D25. This animal subsequently gained weight and survived to the end of the study. Although drug toxicity could not be excluded in this animal, it was unlikely, as no substantial weight loss occurred in other CRLX101-treated mice.

TABLE 12
Response Summary of different treatment regimens in a NSCLC H1299 model.

| | | | | | Response Summary | | | | | | | |
|---|---|---|---|---|---|---|---|---|---|---|---|---|
| | | Treatment | Dose | | | | | p | | | | BW |
| Gr | n | Regimen | (mg/kg) | Schedule | TTE | T − C | % TGD | value | PR | CR | TFS | Nadir |
| 1 | 10 | Vehicle | — | qwk x 3, | 24.4 | — | — | — | 0 | 0 | 0 | — |
| | | | | i.v. | | | | | | | | |
| 2 | 10 | Gemcitabine | 120 | q3d x 4, | 68.7 | 44.3 | 182 | <.001 | 2 | 6 | 5 | −2.3% |
| | | | | i.p. | | | | | | | | |
| 3 | 10 | Docetaxel | 30 | qwk x 3, | 27.8 | 13.4 | 55 | <.001 | 1 | 0 | 0 | −9.3% |
| | | | | i.v. | | | | | | | | |
| 4 | 10 | Topotecan | 12 | q4d x 3, | 39.2 | 14.8 | 61 | <.001 | 0 | 0 | 1 | −0.6% |
| | | | | i.p. | | | | | | | | |
| 5 | 10 | Erlotinib | 100 | qd x 21, | 29.1 | 4.7 | 19 | NS | 0 | 0 | 0 | −2.9% |
| | | | | p.o. | | | | | | | | |
| 6 | 10 | Pemetrexed | 500 | qd x 14, | 26.3 | 1.9 | 8 | NS | 0 | 0 | 0 | — |
| | | | | p.o. | | | | | | | | |
| 7 | 10 | CRLX101 | 6 | Qwk x 3, | 74.0 | 49.6 | 203 | <.001 | 1 | 9 | 9 | — |
| | | | | i.v. | | | | | | | | |
| 8 | 10 | CRLX101 | 10 | qwk x 3, | 74.0 | 49.6 | 203 | <.001 | 0 | 10 | 10 | −1.1% |
| | | | | i.v. | | | | | | | | |

n = number of animals in a group
TTE = time to endpoint
T − C = difference between median TTE (days) of treated versus control group,
% TGD = [(T − C)/C] × 100. The maximum T − C in this study is 49.6 days (203%), compared with Group 1
Statistical Significance NS = not significant,
*P < 0.001 = significant, compared to Group 1
PR = partial regressions;
CR = complete regressions;
TFS = tumor free survivors, i.e., CRs at end of study
BW Nadir = lowest group mean body weight, as % change from Day 1;
“—” indicates that no decrease in mean body weight was observed

### Example 11

### Combination Therapy of CRLX101 and Sorafenib Inhibits Tumor Growth in Non-Small Cell Lung Cancer H1299 Xenograft Model

NCI-H1299 NSCLC cells were obtained from the American Type Culture Collection and were grown in culture to 85-90% confluency in RPMI medium supplemented with 10% FBS and 1% penicillin/streptomycin (passage=4). The cells were then resuspended in RPMI-1640 (no FBS/antibiotics). NCI-H1299 cells (density=50×10⁶ cells in 100 mL) were implanted subcutaneously (SC) into the mammary fat pad of male Taconic NCR nu/nu mice (20-22 g) on day 1.

Five treatments were administered to mice starting 28 days post tumor implantation, when the group mean tumor volume was 350-384 mm3. The animals were divided into the following treatment groups: 1) PBS solution (q7d×3); 2)6 mg/kg CRLX101, weekly for 3 weeks (i.v.) 3) 45 mg/kg sorafenib, daily for 21 days (p.o.); 4); 60 mg/kg sorafenib, daily for 21 days (p.o.); 5) 60 mg/kg sorafenib, daily for 21 days (p.o.) plus 6 mg/kg CRLX101, weekly for 3 weeks (i.v.). Intravenous treatments were given into the tail vein of the mouse at a dose volume of 10 mL/kg. Oral treatments were administered at a dose volume of 10 mL/kg. Health status of the animals was monitored daily and the body weight and tumor volume were measured two times a week for 4 weeks and then once a week thereafter to evaluate the effect of the treatment. The study endpoint used to determine the tumor growth delay was a group mean tumor size of 1000 mm3. Thereafter, the individual endpoint was a mouse tumor size of 1000 mm³, after which the mouse was removed from the study.

### Example 12

### CRLX101 Inhibits HIF in HCT-116 Colorectal Xenograft Tumor Model

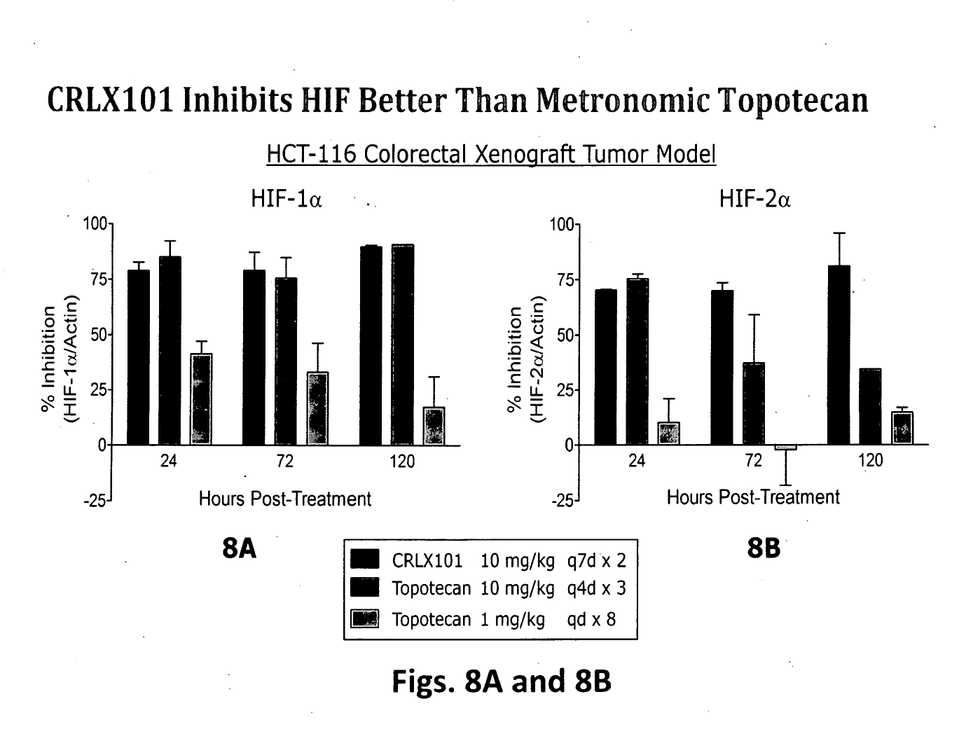

CRLX101 was shown to be a potent inhibitor of HIF-1α, producing a greater and longer lasting inhibition, than metronomic topotecan (FIG. 8A) in an HCT-116 colorectal xenograft tumor model. CRLX101 also exhibited longer lasting inhibition of HIF-2α (FIG. 8B) even after 120 hours as compared to the maximum tolerated doses (MTD) of topotecan and the metronomic topotecan. In addition, the MTD of topotecan out performed metronomic topotecan in inhibiting HIF-1α and HIF-2α.

### Example 13

### HIF-1α/HIF-2α Inhibition in a Panel of Xenografts Tumor Models

Eight different tumor models were grown in nude mice and, when the tumor volumes were in the range of 72-318 mm³, the mice were treated with a single intravenous administration of CRLX101 at 6 mg/kg. Tumor samples were collected 72 hours later and analyzed for HIF-1α and HIF-2α levels using Western blot methods. HIF-1α and HIF-2α levels were compared to control vehicle PBS-treated tumors of the same respective tumor type. HIF-1α and HIF-2α protein levels were normalized to actin protein levels (actin is a protein that is not affected by CRLX101).

Following CRLX101 treatment, HIF-1α and HIF-2α levels declined in 7 of the different tumor models, A2780, SKOV-3, HCT-116, DU-145, H1299, H520 and Caki-1, which represent 5 different tumor types—ovarian, colon, prostate, NSCLC and renal. In one tumor model, the breast cancer tumor model MDA-MB-231/GFP, HIF-1α and HIF-2α levels did not decrease after treatment with CRLX101. Table 13 below contains the data as a percent of PBS-treated control levels.

TABLE 13
HIF-1α and HIF-2α Levels After CRLX101 Treatment

| | HIF-1α | HIF-2α |
|---|---|---|
| Model | as % of Control | as % of Control |
| HCT-116 (colon) | 65 | 24 |
| DU-145 (prostate) | 55 | 58 |
| H1299 (non-small cell lung) | 27 | 7 |
| H520 (non-small cell lung) | 66 | * |
| A2780 (ovarian) | 23 | * |
| SKOV-3 (ovarian) | 27 | 64 |
| Caki-1 (renal) | 56 | 17 |
| MDA-MB-231/GFP (breast) | 92 | * |

* Not measured.

### Example 14

### Inhibition of Downstream Genes in Some Representative Xenograft Models

Eight different tumor models were grown in nude mice and, when the tumor volumes were in the range of 72-318 mm³, the mice were treated with a single intravenous administration of CRLX101 at 6 mg/kg. Tumor samples were collected 72 hours later and analyzed for mRNA levels of the HIF-1α-dependent proteins of VEGF, GLUT-1, GLUT-3, CA9 and CD-31 using qRT-PCR methods. mRNA levels of VEGF, GLUT-1, GLUT-3, CA9 and CD-31 were normalized to mRNA levels of the protein GAPDH (that is not affected by CRLX101) and compared to the respective normalized levels in control vehicle PBS-treated tumors.

Following CRLX 101 treatment, mRNA levels of HIF-1α-dependent proteins declined in the three tumor models analyzed in which there was also a decrease in HIF-1α levels, i.e., A2780, SKOV-3 and HCT-116. In the tumor model that did not respond to CRLX101 with a decrease in HIF-1α levels, the breast cancer tumor model MDA-MB-231/GFP, HIF-1α-dependent protein mRNA levels did not decrease after treatment with CRLX101. Table 14 below contains the data as a percent of PBS-treated control levels.

TABLE 14
HIF-1α Downstream Protein mRNA Levels After CRLX101 Treatment

| | VEGF | GLUT-1 | GLUT-3 | CA9 | CD-31 |
|---|---|---|---|---|---|
| | as % of | as % of | as % of | as % of | as % of |
| Model | Control | Control | Control | Control | Control |
| HCT-116 (colon) | 54 | 70 | 14 | 31 | 11 |
| A2780 (ovarian) | 71 | 74 | 51 | 35 | 72 |
| SKOV-3 (ovarian) | 16 | 70 | 3 | 25 | * |
| MDA-MB-231/GFP | 99 | 172 | 99 | 81 | 111 |
| (breast) | | | | | |

*Not measured

### Example 15

### Kinetics of HIF-1α/HIF-2α Inhibition in the HCT-116 Xenograft Colon Tumor Model

HCT-116 tumors were grown in nude mice and, when the tumor volumes were in the range of 92-246 mm³, the mice were treated with a single intravenous administration of CRLX101 at 6 mg/kg or control vehicle PBS. Tumor samples were collected 24, 72, 120 and 168 hours later and analyzed for HIF-1α and HIF-2α levels using Western blot. HIF-1α and HIF-2α levels were compared to control vehicle PBS-treated tumors. HIF-1α and HIF-2α protein levels were normalized to actin protein levels (actin is a protein that is not affected by CRLX101).

Following CRLX101 treatment, HIF-1α and HIF-2α levels declined to 47% and 50% of PBS-treated control levels at 24 hrs, respectively, and continued to decline to 11% and 9% as compared to PBS-treated control levels at 120 hrs, respectively. At 168 hrs after CRLX101 treatment, HIF-1α and HIF-2α levels remained low, and were 17% and 12% as compared to PBS-treated control levels, respectively. Table 15 below contains the data at the different time points after CRLX101 treatment, as a percent of PBS-treated control levels.

TABLE 15
HIF-1α and HIF-2α Levels After CRLX101 Treatment

| HCT-116 | 24 hr | 72 hr | 120 hr | 168 hr |
|---|---|---|---|---|
| HIF-1α levels as a % of control | 47 | 14 | 11 | 17 |
| HIF-2α levels as a % of control | 50 | 18 | 9 | 12 |

### Example 16

### Synergy of CRLX101 Plus Bevacizumab in Tumor Growth Inhibition and Survival in the A2780 Xenograft Ovarian Tumor Model

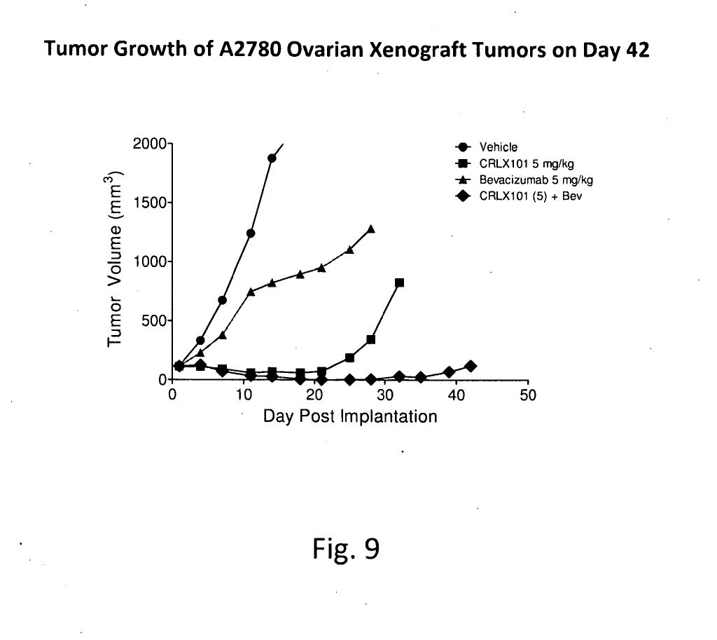

A2780 tumors were grown in nude mice and, when the mean tumor volume was 113.5 mm³, the mice were treated intravenously with either a control vehicle PBS q7d×2 (Day 1, Day 8, Day 15), intravenous CRLX101 at 5 mg/kg IV q7d×3 (Day 1, Day 8, Day 15), intraperitoneal Bevacizumab at 5 mg/kg biweekly×3 (Day 1, Day 4, Day 8, Day 11, Day 14, Day 18) or a combination of both drugs, CRLX101 and Bevacizumab, at their respective doses, routes and schedules. Tumor growth was monitored by measuring the length and width of the tumor, and results are illustrated in FIG. 9. Tumor growth inhibition caused by CRLX101 exceeded the tumor growth inhibition caused by Bevacizumab. The combination of CRLX101 and Bevacizumab showed a synergistic effect on tumor growth inhibition.

FIG. 9 shows the tumor growth curves for CRLX101 and Bevacizumab monotherapy groups. The monotherapy groups did not proceed for the same duration as the combination group because mice within each of the CRLX101 and Bevacizumab monotherapy groups reached the endpoint of 2000 mm³ and were removed from the study, leaving too few mice in these groups to represent a treatment effect on tumor growth beyond Day 32.

When CRLX101 was combined with Bevacizumab, the response rate also showed synergy, with 6 mice being completely tumor-free in this treatment group. Table 16 below compares response rates of each treatment group.

TABLE 16
Response Rate as Measured by Tumor Volume of A2780
Ovarian Xenograft Tumors on Day 42

| | | Partial | Complete | |
|---|---|---|---|---|
| | | Response | Response | Tumor Free |
| Group | Treatment | (# of mice) | (# of mice) | (# of mice) |
| 1 | Vehicle control | 0 of 10 | 0 of 10 | 0 of 10 |
| 2 | CRLX101 | 3 of 10 | 3 of 10 | 2 of 10 |
| 3 | Bevacizumab | 0 of 10 | 2 of 10 | 2 of 10 |
| 4 | CRLX101 + BEV | 1 of 9 | 8 of 9 | 6 of 9 |

### Example 17

### Synergy of CRLX101 Plus Bevacizumab in HIF-1α and HIF-2α Inhibition in the A2780 Xenograft Ovarian Tumor Model

A2780 tumors were grown in nude mice and, when the mean tumor volume was 109 mm³, the mice were treated intravenously with a control vehicle PBS q7d×2 (Day 1 and Day 8), intravenous CRLX101 at 5 mg/kg q7d×2 (Day 1 and Day 8), intraperitoneal Bevacizumab at 5 mg/kg biweekly×2 (Day 1, Day 4 and Day 8), or a combination of both drugs, CRLX101 and Bevacizumab, at their respective doses, routes and schedules. Tumor samples were collected on Day 11, 72 hours after the last treatment (Day 8 for both drugs) and analyzed for HIF-1α and HIF-2α levels using Western blot. HIF-1α and HIF-2α protein levels were normalized to actin protein levels (actin is not affected by CRLX101) and HIF-1α and HIF-2α protein levels were compared to levels of control vehicle PBS-treated tumors.

CRLX101 caused a decrease in HIF-1α and HIF-2α levels to 76% and 65% as compared to PBS-treated control levels, respectively. Bevacizumab caused an increase in HIF-1α and HIF-2α levels to 157% and 146% as compared to PBS-treated control levels, respectively. The combination of CRLX101 and Bevacizumab showed a synergistic effect for both HIF-1α and HIF-2α, with declines to 25% and 17% of the HIF-1α and HIF-2α levels in the CRLX101 treatment group, respectively, as compared to PBS-treated control levels. Table 17 below contains the data for each treatment group. The HIF-1α and HIF-2α levels for CRLX101, 76% and 65%, respectively, are as a percent of PBS-treated control levels. The HIF-1α and HIF-2α levels for Bevacizumab, 157% and 146%, respectively, are as a percent of PBS-treated control levels. The HIF-1α and HIF-2α levels for CRLX101 and Bevacizumab in combination are as a percent of PBS-treated control levels (19% and 11%, respectively) and as a percent of CRLX101 monotherapy (25% and 17%, respectively).

TABLE 17
HIF-1α and HIF-2α Levels After CRLX101 and Bevacizumab
Treatment as Monotherapies and in Combination

| | | HIF-1α | HIF-2α | HIF-1α | HIF-2α |
|---|---|---|---|---|---|
| | Dose | % of | % of | % of | % of |
| Treatment | mg/kg | Control | Control | CRLX101 | CRLX101 |
| Vehicle control | * | 100 | 100 | * | * |
| CRLX101 | 5 | 76 | 65 | 100 | 100 |
| Bevacizumab | 5 | 157 | 146 | * | * |
| CRLX101 + BEV | 5 + 5 | 19 | 11 | 25 | 17 |

* Not measured.

Other embodiments are in the claims.

We claim:

1. A method of treating a proliferative disorder in a subject, comprising:
administering a CDP-topoisomerase inhibitor conjugate, particle or composition, e.g., CRLX101, in combination with an angiogenesis inhibitor, e.g., a VEGF pathway inhibitor, e.g., a VEGF pathway inhibitor described herein, e.g., a VEGF inhibitor, e.g., a small molecule inhibitor, protein, e.g., a fusion protein (e.g., aflibercept) or an antibody against VEGF, e.g., bevacizumab; or a VEGF receptor inhibitor (e.g., a VEGF receptor 1 inhibitor or a VEGF receptor 2 inhibitor), e.g., a small molecule inhibitor, e.g., sorafenib, sunitinib, pazopanib or brivanib, or an antibody against VEGF receptor at a dosing schedule described herein.
